(19) United States
(12) Patent Application Publication
(10) Pub. No.: US 2014/0121231 A1
(43) Pub. Date: May 1, 2014

(54) PYRANOPYRIDONE INHIBITORS OF TANKYRASE

(71) Applicant: Hoffmann-La Roche, Inc., South San Francisco, CA (US)

(72) Inventors: David Robert Bolin, Montclair, NJ (US); Javier de Vicente Fidalgo, Foster City, CA (US); Johannes Hermann, Jersey City, NJ (US); Parcharee Tivitmahaisoon, Lyndhurst, NJ (US); Lin Yi, Basking Ridge, NJ (US); Mark Zak, San Mateo, CA (US)

(21) Appl. No.: 13/921,937

(22) Filed: Jun. 19, 2013

Related U.S. Application Data
(60) Provisional application No. 61/661,915, filed on Jun. 20, 2012.

(51) Int. Cl. C07D 491/052; C07D 491/153; C07D 491/107; C07D 491/147; C07D 495/14

(52) U.S. Cl. CPC C07D 491/052; C07D 491/147; C07D 495/14; C07D 491/107; C07D 491/153
USPC 514/278; 546/89; 514/291; 546/83; 514/293; 546/15; 546/90

(57) ABSTRACT

There are provided compounds of the formula

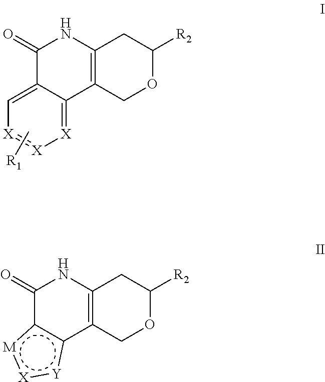

or a pharmaceutically acceptable salt thereof wherein X, M, Y, R₁ and R₂ are as defined herein.

The compounds have activity as anticancer agents.

### CROSS REFERENCE TO PRIOR APPLICATIONS

The application claims the benefit of priority to U.S. Ser. No. 61/661,915 filed Jun. 20, 2012 the contents of which is incorporated herein by reference in its entirety.

### FIELD OF THE INVENTION

The present invention relates to pyranopyridones which act as inhibitors of the enzyme tankyrase and are useful in the amelioration or treatment of cancer.

### BACKGROUND OF THE INVENTION

Cancer is a disease characterized by the loss of appropriate control for cell growth. The American Cancer Society has estimated that there were in excess of 1.5 million new cases of cancer within the United Stated of America in 2010 and approximately 570,000 deaths that year estimated to be attributable to cancer. The World Health Organization has estimated that cancer was the leading cause of death globally in 2010, with the number of deaths caused by cancer growing to 12 million per year by 2030.

It has been suggested that there are 6 capabilities which need to be developed by cells in order to lead to the formation of cancerous lesions. These traits are self-sufficiency in growth signals, insensitivity to anti-growth signals, tissue invasion and metastasis, limitless replication potential, sustained angiogenesis and evasion of apoptosis. Growth signaling is required for cells to transition from a quiescent state into an active proliferative state. These signals are typically transmitted from transmembrane receptors, through signal transduction cascades involving numerous intracellular kinases, eventually resulting in changes in gene expression at the nuclear level within the cell. In recent years there has been much interest in the area of signal transduction inhibitors, particularly kinase inhibitors, and their use for the treatment of cancer. Several examples from this class of compounds have been successfully evaluated in clinical settings and are now commercially available and marketed for the treatment of specific forms of cancer e.g. imatinib tosylate (marketed as Gleevec® by Novartis for the treatment of Philadelphia chromosome-positive chronic myeloid leukemia), lapatinib ditosylate (marketed as Tykerb® by GlaxoSmithKline for the treatment of HER2 positive breast cancer in combination with other chemotherapeutic agents), sunitinib malate (marketed as Sutent® by Pfizer and approved for the treatment of renal cancer) and sorafenib (marketed as Nexavar by Bayer for the treatment of renal cancer).

In addition to the growth factor associated signaling pathways, which predominantly utilize kinase catalyzed transfer of phosphate groups as the key component of the signaling pathway, numerous other signaling pathways also exist within cells and their proper regulation is critical for maintaining correct levels of cell growth and replication. In the emerging area of cancer stem cell inhibition the Wnt, Notch and Hedgehog pathways have received much interest as potential ways in which to avoid tumor relapse and metastasis. The Wnt pathway is instrumental in embryonic development and in tissue maintenance in adults with the activity of individual components within the pathway under tight regulation. In cancer and other diseases cell signaling pathways no longer exhibit the appropriate level of control. In the case of the Wnt pathway, signal transduction is controlled by the relative stabilities of 2 proteins, axin and β-catenin. An overabundance of β-catenin leads to increased Wnt signaling and activation of associated nuclear transcription factors while excess axin results in the degradation of intracellular β-catenin and decreased signaling. Dysregulation of the canonical Wnt signaling pathway has been implicated in a range of human carcinomas such as colon cancer, hepatocellular carcinoma, endometrial ovarian cancer, pilomatricoma skin cancer, prostate cancer, melanoma and Wilms tumor.

In the canonical Wnt signaling pathway signaling is initiated by interaction of a Wnt ligand with a receptor complex containing a Frizzled family member and low-density lipoprotein receptor-related protein. This leads to the formation of a disheveled-frizzled complex and relocation of axin from the destruction complex to the cell membrane. Axin is the concentration limiting component of the destruction complex, and it is this complex which is formed with adenomatous polyposis coli proteins, casein-kinase 1α and glycogen synthase kinase 3β which is responsible for controlling intracellular levels of β-catenin. In the presence of functional destruction complex, β-catenin is sequentially phosphorylated by casein-kinase 1α and glycogen synthase kinase 3β on a conserved set of serine and threonine residues at the amino-terminus. Phosphorylation facilitates binding of β-catenin to β-transducin repeat-containing protein which then mediates ubiquitination and subsequent proteasomal degradation of β-catenin. In the absence of sufficiently elevated concentrations of the destruction complex, un-phosphorylated β-catenin is able to migrate to the cell nucleus and interact with T-cell factor proteins and convert them into potent transcriptional activators through the recruitment of co-activator proteins.

It has recently been reported that intracellular axin levels are influenced by the poly(ADP-ribose) polymerase enzyme family members tankyrase-1 and tankyrase-2 (also known as PARP5a and PARP5b) (Nature Chemical Biology 2009 5:100 and Nature 2009 461:614). Tankyrase enzymes are able to poly-ADP ribosylate (PARsylate) axin, which marks this protein for subsequent ubiquitination and proteasomal degradation. Thus, it would be expected that in the presence of an inhibitor of tankyrase catalytic activity, axin protein concentration would be increased, resulting in higher concentration of the destruction complex and decreased concentrations of unphosphorylated intracellular β-catenin and decreased Wnt signaling. An inhibitor of tankyrase-1 and -2 would also be expected to have an effect on other biological functions of the tankyrase proteins e.g. chromosome end protection (telomeres), insulin responsiveness and spindle assembly during mitosis (Biochimie 2009 5:100).

Therapeutics which are directed at and can correct dysregulation of the Wnt signaling pathway have been implicated in conditions such as bone density defects, coronary disease, late onset Alzheimer's disease, familial exudative vitreoretinopathy, retinal angiogenesis, tetra-amelia, Mullerian-duct regression and virilization, SERKAL syndrome, type 2 diabetes, Fuhrmann syndrome, skeletal dysplasia, focal dermal hypoplasia and neural tube defects. Although the above introduction has focused on the relevance of Wnt signaling in cancer, the Wnt signaling pathway is of fundamental importance and has potential implication in a broad range of human diseases, not necessarily limited to the examples provided above for illustrative purposes.

### SUMMARY OF THE INVENTION

There is a continuing need for new and novel therapeutic agents that can be used for cancer and hyperproliferative conditions. The tankyrase enzymes, which modulate Wnt activity, are members of the PARP family. Design and development of new pharmaceutical compounds that inhibit or modulate their activity is essential. In one aspect of the present invention there is provided a compound according to formula I and formula II.

One aspect of the invention is a compound of formulas I or II wherein:

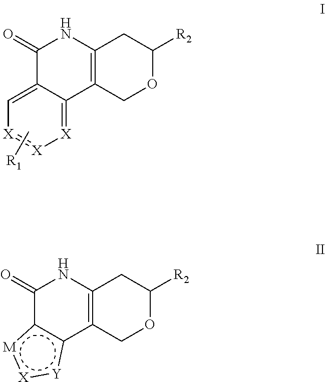

X is independently in each occurrence N or CH,

### Y is S, O, CH or NCH₃,

### M is S or CH,

R₁ is H, alkyl, cycloalkyl, C(CH₃)₂OH, CN, nitro, CO₂CH₃, CONH₂, NH₂, or halogen, and,
R₂ is selected from the group consisting of H, optionally substituted C₁₋₆ alkyl, C₅₋₁₂ spiroalkyl, C₁₋₆ alkoxy, C₃₋₇ cycloalkyl, heterocycloalkyl and substituted heterocycloalkyl wherein said heterocycloalkyl is optionally substituted by C₁₋₆ alkyl, C₁₋₆ hydroxyalkyl, C₁₋₃ alkoxy-C₁₋₆ alkyl, oxetanyl, tetrahydrofuranyl, pyranyl or SO₂R₃ wherein R₃ is C₁₋₆ alkyl, C₁₋₆ hydroxyalkyl, oxetanyl, tetrahydrofuranyl, pyranyl; or, a pharmaceutically acceptable salt thereof.

The present invention additionally relates to pharmaceutical compositions comprising one or more compounds of the invention, or a pharmaceutically acceptable salt, and a pharmaceutically acceptable carrier or excipient.

The present invention further relates to a method of treating, ameliorating or preventing cancer in a mammal, preferably a human, comprising administering to said mammal a therapeutically effective amount of a compound according to the invention or a pharmaceutically acceptable salt thereof.

### DETAILED DESCRIPTION OF THE INVENTION

The phrase “a” or “an” entity as used herein refers to one or more of that entity; for example, a compound refers to one or more compounds or at least one compound. As such, the terms “a” (or “an”), “one or more”, and “at least one” can be used interchangeably herein.

The phrase “as defined herein above” refers to the broadest definition for each group as provided in the Summary of the Invention or the broadest claim. In all other embodiments provided below, substituents which can be present in each embodiment and which are not explicitly defined retain the broadest definition provided in the Summary of the Invention.

As used in this specification, whether in a transitional phrase or in the body of the claim, the terms “comprise(s)” and “comprising” are to be interpreted as having an open-ended meaning. That is, the terms are to be interpreted synonymously with the phrases “having at least” or “including at least”. When used in the context of a process, the term “comprising” means that the process includes at least the recited steps, but may include additional steps. When used in the context of a compound or composition, the term “comprising” means that the compound or composition includes at least the recited features or components, but may also include additional features or components.

The term “independently” is used herein to indicate that a variable is applied in any one instance without regard to the presence or absence of a variable having that same or a different definition within the same compound. Thus, in a compound in which R″ appears twice and is defined as “independently carbon or nitrogen”, both R″s can be carbon, both R″s can be nitrogen, or one R″ can be carbon and the other nitrogen.

When any variable (e.g., R¹, R^{4a}, Ar, X¹ or Het) occurs more than one time in any moiety or formula depicting and describing compounds employed or claimed in the present invention, its definition on each occurrence is independent of its definition at every other occurrence. Also, combinations of substituents and/or variables are permissible only if such compounds result in stable compounds.

The symbols “*” at the end of a bond or “ [symbol] ” drawn through a bond each refer to the point of attachment of a functional group or other chemical moiety to the rest of the molecule of which it is a part. Thus, for example:

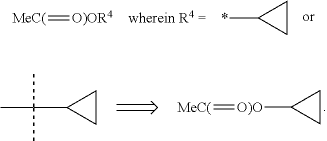

A bond drawn into ring system (as opposed to connected at a distinct vertex) indicates that the bond may be attached to any of the suitable ring atoms.

The term “optional” or “optionally” as used herein means that a subsequently described event or circumstance may, but need not, occur, and that the description includes instances where the event or circumstance occurs and instances in which it does not. For example, “optionally substituted” means that the optionally substituted moiety may incorporate a hydrogen or a substituent.

The term “about” is used herein to mean approximately, in the region of, roughly, or around. When the term “about” is used in conjunction with a numerical range, it modifies that range by extending the boundaries above and below the numerical values set forth. In general, the term “about” is used herein to modify a numerical value above and below the stated value by a variance of 20%.

As used herein, the recitation of a numerical range for a variable is intended to convey that the invention may be practiced with the variable equal to any of the values within that range. Thus, for a variable which is inherently discrete, the variable can be equal to any integer value of the numerical range, including the end-points of the range. Similarly, for a variable which is inherently continuous, the variable can be equal to any real value of the numerical range, including the end-points of the range. As an example, a variable which is described as having values between 0 and 2, can be 0, 1 or 2 for variables which are inherently discrete, and can be 0.0, 0.1, 0.01, 0.001, or any other real value for variables which are inherently continuous.

In one embodiment of the present invention there is provided a compound according to formula I or II wherein X, Y, M, R₁, R₂ and R₃ are as defined hereinabove.

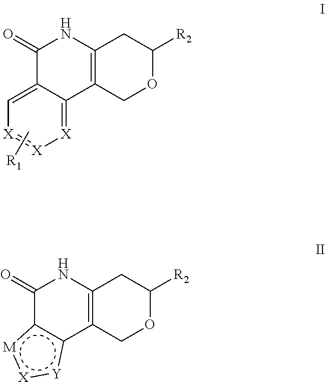

In one embodiment of the present invention there is provided a compound according to formula I or II wherein X is N or CH, Y is S, O, CH or NCH₃, M is S or CH, R₁ is H, alkyl, cycloalkyl, C(CH₃)₂OH, CN, nitro, CO₂CH₃, CONH₂, NH₂, or halogen; and, R₂ is selected from the group consisting of H, alkyl, substituted alkyl, spiroalkyl, alkoxy, cycloalkyl, heterocycloalkyl and substituted heterocycloalkyl; or a pharmaceutically acceptable salt thereof.

In another embodiment of the present invention there is provided a compound according to formula I wherein X is CH or N; R₁ is H or CH₃ and R₂ is selected from C₁₋₆ alkyl, substituted C₁₋₆ alkyl or substituted heterocycloalkyl.

In another embodiment of the present invention there is provided a compound according to formula I wherein one X is N and the others are CH; R₁ is H or CH₃; and R₂ is selected from alkyl, substituted alkyl or substituted heterocycloalkyl.

In another embodiment of the present invention there is provided a compound according to formula II wherein one of M or Y is S and the other of M or Y and X are CH; R₁ is H or CH₃ and R₂ is selected from C₁₋₆ alkyl, substituted C₁₋₆ alkyl or substituted heterocycloalkyl.

In another embodiment of the present invention there is provided a compound according to formula II wherein M is CH, X is N, Y is NMe; R₁ is H or CH₃ and R₂ is selected from C₁₋₆ alkyl, substituted C₁₋₆ alkyl or substituted heterocycloalkyl.

In another embodiment of the present invention there is provided a compound according to formula I wherein X is CH or N; R₁ is H or CH₃ and R₂ is selected from C₁₋₆ alkyl, substituted C₁₋₆ alkyl or piperidin-4-yl optionally substituted by C₁₋₆ alkyl, C₁₋₆ hydroxyalkyl, C₁₋₃ alkoxy-C₁₋₆ alkyl, oxetanyl, tetrahydrofuranyl, pyranyl or SO₂R₃ wherein R₃ is C₁₋₆ alkyl, C₁₋₆ hydroxyalkyl, oxetanyl, tetrahydrofuranyl, pyranyl.

In another embodiment of the present invention there is provided a compound according to formula I wherein X is CH or N; R₁ is H or CH₃ and R₂ is piperidin-4-yl optionally substituted substituted by C₁₋₆ alkyl, C₁₋₆ hydroxyalkyl, C₁₋₃ alkoxy-C₁₋₆ alkyl, oxetanyl, tetrahydrofuranyl, pyranyl or SO₂R₃ wherein R₃ is C₁₋₆ alkyl, C₁₋₆ hydroxyalkyl, oxetanyl, tetrahydrofuranyl, pyranyl.

In another embodiment of the present invention there is provided a compound according to formula I wherein one X is N and the others are CH; R₁ is H or CH₃ and R₂ is piperidin-4-yl optionally substituted by C₁₋₆ alkyl, C₁₋₆ hydroxyalkyl, C₁₋₃ alkoxy-C₁₋₆ alkyl, oxetanyl, tetrahydrofuranyl, pyranyl or SO₂R₃ wherein R₃ is C₁₋₆ alkyl, C₁₋₆ hydroxyalkyl, oxetanyl, tetrahydrofuranyl, pyranyl.

In another embodiment of the present invention there is provided a compound according to formula I wherein X is CH or N; R₁ is H or CH₃ and R₂ is alkyl. In a subembodiment the alkyl group is tert-butyl. In another subembodiment the alkyl group is isopropyl. In yet another embodiment R₂ is C₁₋₆ alkylsubstituted by a hydroxyl.

In one embodiment of the present invention there is provided one or more compound(s) selected from I-1 to I-68 of TABLE 1. In another embodiment of the present invention there is provided one or more compound(s) from I-69 to I-76 of TABLE 1. In yet another embodiment of the present invention there is provided one or more compound(s) from I-1 to I-76 of TABLE 1

In another embodiment of the present invention there is provided a compound according to formula I or formula II selected from the following:
rac-2-Isopropyl-1,2,4,10-tetrahydro-3-oxa-10-aza-phenanthren-9-one,
1,2,4,10-Tetrahydro-3-oxa-10-aza-phenanthren-9-one,
rac-2-Isopropyl-1,2,4,10-tetrahydro-3-oxa-5,10-diaza-phenanthren-9-one,
rac-7-Isopropyl-5,6,7,9-tetrahydro-8-oxa-3-thia-5-aza-cyclopenta[a]naphthalen-4-one,
rac-7-Chloro-2-isopropyl-1,2,4,10-tetrahydro-3-oxa-10-aza-phenanthren-9-one,
rac-2-Cyclopentyl-1,2,4,10-tetrahydro-3-oxa-10-aza-phenanthren-9-one,
rac-2-tert-Butyl-1,2,4,10-tetrahydro-3-oxa-10-aza-phenanthren-9-one,
1′,5′-Dihydrospiro[cyclopentane-1,3′-pyrano[4,3-c]isoquinolin]-6′(4′H)-one,
rac-7-Isopropyl-5,6,7,9-tetrahydro-1,8-dioxa-5-aza-cyclopenta[a]naphthalen-4-one,
(S)-2-Cyclopentyl-1,2,4,10-tetrahydro-3-oxa-10-aza-phenanthren-9-one,
(R)-2-Cyclopentyl-1,2,4,10-tetrahydro-3-oxa-10-aza-phenanthren-9-one and
rac-2-Phenethyl-1,2,4,10-tetrahydro-3-oxa-10-aza-phenanthren-9-one.

In another embodiment of the present invention there is provided the compound according to formula I or formula II selected from the following:
rac-7-Isopropyl-5,7,8,9-tetrahydro-6-oxa-3,9-diaza-phenanthren-10-one,
rac-7-Isopropyl-5,7,8,9-tetrahydro-6-oxa-2,9-diaza-phenanthren-10-one,
rac-2-Isopropyl-6-nitro-1,2,4,10-tetrahydro-3-oxa-10-aza-phenanthren-9-one,
rac-2-Isopropyl-5-methyl-1,2,4,10-tetrahydro-3-oxa-10-aza-phenanthren-9-one,
2-sec-Butyl-1,2,4,10-tetrahydro-3-oxa-10-aza-phenanthren-9-one,
rac-2-Isopropyl-9-oxo-1,4,9,10-tetrahydro-2H-3-oxa-10-aza-phenanthrene-6-carboxylic acid methyl ester,
rac-2-(1-Methyl-cyclohexyl)-1,2,4,10-tetrahydro-3-oxa-10-aza-phenanthren-9-one,
rac-4-(9-Oxo-1,4,9,10-tetrahydro-2H-3-oxa-10-aza-phenanthren-2-yl)-piperidine-1-carboxylic acid tert-butyl ester,
rac-2-Piperidin-4-yl-1,2,4,10-tetrahydro-3-oxa-10-aza-phenanthren-9-one trifluoroacetate,
rac-2-Isopropyl-9-oxo-1,4,9,10-tetrahydro-2H-3-oxa-10-aza-phenanthrene-6-carboxylic acid amide,
rac-6-Amino-2-isopropyl-1,2,4,10-tetrahydro-3-oxa-10-aza-phenanthren-9-one and
rac-2-(1-Acetyl-piperidin-4-yl)-1,2,4,10-tetrahydro-3-oxa-10-aza-phenanthren-9-one.

In another embodiment of the present invention there is provided the compound according to formula I or formula II selected from the following:
rac-2-(1-Methanesulfonyl-piperidin-4-yl)-1,2,4,10-tetrahydro-3-oxa-10-aza-phenanthren-9-one,
rac-(2-{2-[2-(2-{3-Oxo-3-[4-(9-oxo-1,4,9,10-tetrahydro-2H-3-oxa-10-aza-phenanthren-2-yl)-piperidin-1-yl]-propoxy}-ethoxy)-ethoxy]-ethoxy}-ethyl)-carbamic acid tert-butyl ester,
rac-6-Chloro-2-isopropyl-1,2,4,10-tetrahydro-3-oxa-10-aza-phenanthren-9-one,
rac-7-Isopropyl-5,6,7,9-tetrahydro-8-oxa-1-thia-5-aza-cyclopenta[a]naphthalen-4-one,
rac-2-(2-Benzyloxy-1,1-dimethyl-ethyl)-1,2,4,10-tetrahydro-3-oxa-10-aza-phenanthren-9-one,
rac-2-(2-Hydroxy-1,1-dimethyl-ethyl)-1,2,4,10-tetrahydro-3-oxa-10-aza-phenanthren-9-one,
rac-7-Cyclopentyl-5,7,8,9-tetrahydro-6-oxa-3,9-diaza-phenanthren-10-one,
rac-4-(9-Oxo-1,4,9,10-tetrahydro-2H-3-oxa-10-aza-phenanthren-2-yl)-piperidine-1-carboxylic acid isopropyl ester,
rac-2-(1-Cyclopropanecarbonyl-piperidin-4-yl)-1,2,4,10-tetrahydro-3-oxa-10-aza-phenanthren-9-one,
rac-7-tert-Butyl-4a,5,7,8,9,10a-hexahydro-6-oxa-3,9-diaza-phenanthren-10-one,
3-(9-Oxo-1,4,9,10-tetrahydro-2H-3-oxa-10-aza-phenanthren-2-yl)-pyrrolidine-1-carboxylic acid tert-butyl ester and
2-Pyrrolidin-3-yl-1,2,4,10-tetrahydro-3-oxa-10-aza-phenanthren-9-one hydrochloride.

In another embodiment of the present invention there is provided there a compound according to formula I or formula II selected from the following:
2-(1-Acetyl-pyrrolidin-3-yl)-1,2,4,10-tetrahydro-3-oxa-10-aza-phenanthren-9-one,
rac-6-Cyclopropyl-2-isopropyl-1,2,4,10-tetrahydro-3-oxa-10-aza-phenanthren-9-one,
2-(1-Methanesulfonyl-pyrrolidin-3-yl)-1,2,4,10-tetrahydro-3-oxa-10-aza-phenanthren-9-one,
rac-7-tert-Butyl-5,6,7,9-tetrahydro-8-oxa-3-thia-5-aza-cyclopenta[a]naphthalen-4-one,
rac-2-tert-Butyl-5-methyl-1,2,4,10-tetrahydro-3-oxa-10-aza-phenanthren-9-one,
rac-7-tert-Butyl-1-methyl-5,6,7,9-tetrahydro-1H-8-oxa-1,2,5-triaza-cyclopenta[a]naphthalen-4-one,
rac-7-tert-Butyl-5,7,8,9-tetrahydro-6-oxa-2,9-diaza-phenanthren-10-one,
2-(1-Cyclopropanecarbonyl-pyrrolidin-3-yl)-1,2,4,10-tetrahydro-3-oxa-10-aza-phenanthren-9-one,
2-[1-(1-Methyl-cyclopropanecarbonyl)-pyrrolidin-3-yl]-1,2,4,10-tetrahydro-3-oxa-10-aza-phenanthren-9-one,
rac-2-[1-(1-Methyl-cyclopropanecarbonye-piperidin-4-yl]-1,2,4,10-tetrahydro-3-oxa-10-aza-phenanthren-9-one,
rac-2-(Tetrahydro-pyran-4-yl)-1,2,4,10-tetrahydro-3-oxa-10-aza-phenanthren-9-one and
rac-2-tert-Butyl-1,2,4,10-tetrahydro-3-oxa-5,10-diaza-phenanthren-9-one.

In another embodiment of the present invention there is provided a compound according to claim 1 selected from the following:
rac-2-tert-Butyl-6-methyl-1,2,4,10-tetrahydro-3-oxa-5,10-diaza-phenanthren-9-one,
rac-2-[1-(Tetrahydro-pyran-4-carbonyl)-piperidin-4-yl]-1,2,4,10-tetrahydro-3-oxa-10-aza-phenanthren-9-one,
rac-7-Isopropyl-5,7,8,9-tetrahydro-6-oxa-2,9-diaza-phenanthren-10-one,
rac-2-[1-(Oxetane-3-carbonyl)-piperidin-4-yl]-1,2,4,10-tetrahydro-3-oxa-10-aza-phenanthren-9-one,
rac-2-tert-Butyl-6-(1-hydroxy-1-methyl-ethyl)-1,2,4,10-tetrahydro-3-oxa-10-aza-phenanthren-9-one,
4-((S)-9-Oxo-1,4,9,10-tetrahydro-2H-3-oxa-10-aza-phenanthren-2-yl)-piperidine-1-carboxylic acid tert-butyl ester,
4-((R)-9-Oxo-1,4,9,10-tetrahydro-2H-3-oxa-10-aza-phenanthren-2-yl)-piperidine-1-carboxylic acid tert-butyl ester,
rac-2-tert-Butyl-9-oxo-1,4,9,10-tetrahydro-2H-3-oxa-10-aza-phenanthrene-6-carbonitrile,
rac-2-(2-Hydroxy-1,1-dimethyl-ethyl)-5-methyl-1,2,4,10-tetrahydro-3-oxa-10-aza-phenanthren-9-one,
rac-2-[1-(1-methyl-cyclopropanecarbonyl)-piperidin-4-yl]-1,2,4,10-tetrahydro-3-oxa-5,10-diaza-phenanthren-9-one,
rac-7-tert-Butyl-4-methyl-5,7,8,9-tetrahydro-6-oxa-2,9-diaza-phenanthren-10-one,
2-(Tetrahydro-furan-3-yl)-1,2,4,10-tetrahydro-3-oxa-10-aza-phenanthren-9-one and
rac-5-Methyl-2-(tetrahydro-pyran-4-yl)-1,2,4,10-tetrahydro-3-oxa-10-aza-phenanthren-9-one.

In another embodiment of the present invention there is provided a compound according to formula I or formula II selected from the following:
rac-5-Methyl-2-[1-(1-methyl-cyclopropanecarbonyl)-piperidin-4-yl]-1,2,4,10-tetrahydro-3-oxa-10-aza-phenanthren-9-one,
rac-4-(5-Methyl-9-oxo-1,4,9,10-tetrahydro-2H-3-oxa-10-aza-phenanthren-2-yl)-piperidine-1-carboxylic acid tert-butyl ester,
rac-2-(1-Cyclopropanecarbonyl-piperidin-4-yl)-5-methyl-1,2,4,10-tetrahydro-3-oxa-10-aza-phenanthren-9-one,
rac-2-(1-Hydroxy-1-methyl-ethyl)-5-methyl-1,2,4,10-tetrahydro-3-oxa-10-aza-phenanthren-9-one,
rac-2-(3-Methyl-oxetan-3-yl)-1,2,4,10-tetrahydro-3-oxa-10-aza-phenanthren-9-one,
(S)-2-(2-Hydroxy-1,1-dimethyl-ethyl)-5-methyl-1,2,4,10-tetrahydro-3-oxa-10-aza-phenanthren-9-one and
(R)-2-(2-Hydroxy-1,1-dimethyl-ethyl)-5-methyl-1,2,4,10-tetrahydro-3-oxa-10-aza-phenanthren-9-one.

In another embodiment of the present invention there is provided a pharmaceutical composition containing a compound according to formula I or II wherein X, Y, M, R₁, R₂ and R₃ are as defined hereinabove and at least one pharmaceutically acceptable carrier, diluent or excipient.

In another embodiment of the present invention there is provided a pharmaceutical composition containing a compound according to formula I or II selected from compounds I-1 to I-76 of TABLE 1 and at least one pharmaceutically acceptable carrier, diluent or excipient.

In another embodiment of the present invention there is provided a pharmaceutical composition containing a compound selected from compounds I-1 to I-68 of TABLE 1 and at least one pharmaceutically acceptable carrier, diluent or excipient.

In another embodiment of the present invention there is provided a compound according to formula I or II wherein X, Y, M, R₁, R₂ and R₃ are as defined hereinabove for use in the treatment of cancer.

In another embodiment of the present invention there is provided a compound according to formula I or II wherein X, Y, M, R₁, R₂ and R₃ are as defined hereinabove for the preparation of a medicament for the treatment of cancer.

In another embodiment of the present invention there is provided a method for the treatment of cancer comprising administration of an effective amount of a compound according to formula I or II wherein X, Y, M, R₁, R₂ and R₃ are as defined hereinabove.

As used herein, the following terms shall have the following definitions.

The term “alkyl” refers to straight- or branched-chain saturated hydrocarbon groups having from 1 to about 12 carbon atoms, including groups having from 1 to about 7 carbon atoms. In certain embodiments, alkyl substituents may be lower alkyl substituents. The term “lower alkyl” refers to alkyl groups having from 1 to 6 carbon atoms, preferably from 1 to 4 carbon atoms. Examples of alkyl groups include, but are not limited to, methyl, ethyl, n-propyl, i-propyl, n-butyl, s-butyl, t-butyl, n-pentyl, and s-pentyl.

The term “alkenyl” as used herein means an unsaturated straight-chain or branched aliphatic hydrocarbon group containing at least one double bond and having 2 to 6, preferably 2 to 4 carbon atoms. Examples of such “alkenyl group” are vinyl, ethenyl, allyl, isopropenyl, 1-propenyl, 2-methyl-1-propenyl, 1-butenyl, 2-butenyl, 3-butenyl, 2-ethyl-1-butenyl, 3-methyl-2-butenyl, 1-pentenyl, 2-pentenyl, 3-pentenyl, 4-pentenyl, 4-methyl-3-pentenyl, 1-hexenyl, 2-hexenyl, 3-hexenyl, 4-hexenyl and 5-hexenyl.

“Alkoxy, alkoxyl or lower alkoxy” refers to any of the above lower alkyl groups which is attached to the remainder of the molecule by an oxygen atom (RO—). Typical lower alkoxy groups include methoxy, ethoxy, isopropoxy or propoxy, butyloxy and the like. Further included within the meaning of alkoxy are multiple alkoxy side chains, e.g. ethoxy ethoxy, methoxy ethoxy, methoxy ethoxy ethoxy and the like and substituted alkoxy side chains, e.g., dimethylamino ethoxy, diethylamino ethoxy, dimethoxy-phosphoryl methoxy and the like.

The term “alkynyl” as used herein means an unsaturated straight-chain or branched aliphatic hydrocarbon group containing one triple bond and having 2 to 6, preferably 2 to 4 carbon atoms. Examples of such “alkynyl group” are ethynyl, 1-propynyl, 2-propynyl, 1-butynyl, 2-butynyl, 3-butynyl, 1-pentynyl, 2-pentynyl, 3-pentynyl, 4-pentynyl, 1-hexynyl, 2-hexynyl, 3-hexynyl, 4-hexynyl and 5-hexynyl.

Amino means the group —NH₂.

“Aryl” means a monovalent, monocyclic or bicyclic, aromatic carboxylic

hydrocarbon radical, preferably a 6-10 member aromatic ring system. Preferred aryl groups include, but are not limited to, phenyl, naphthyl, tolyl, and xylyl.

Carboxyl or carboxy means the monovalent group —COOH. Carboxy lower alkyl means —COOR, wherein R is lower alkyl. Carboxy lower alkoxy means —COOROH wherein the R is lower alkyl.

Carbonyl means the group RC(═O)R′, where R′ and R″ independently can be any of a number of chemical groups including alkyl.

The term “cycloalkyl” as used herein means any stable monocyclic or polycyclic system which consists of carbon atoms only, any ring of which being saturated, and the term “cycloalkenyl” is intended to refer to any stable monocyclic or polycyclic system which consists of carbon atoms only, with at least one ring thereof being partially unsaturated. Examples of cycloalkyls include, but are not limited to, cyclopropyl, cyclobutyl, cyclopentyl, cyclohexyl, cycloheptyl, adamantyl, cyclooctyl, bicycloalkyls, including bicyclooctanes such as [2.2.2]bicyclooctane or [3.3.0]bicyclooctane, bicyclononanes such as [4.3.0]bicyclononane, and bicyclodecanes such as [4.4.0]bicyclodecane (decalin), or spiro compounds. Examples of cycloalkenyls include, but are not limited to, cyclopentenyl or cyclohexenyl.

The term “halogen” as used herein means fluorine, chlorine, bromine, or iodine, preferably fluorine and chlorine.

“Heteroaryl” means an aromatic heterocyclic ring system containing up to two rings. Preferred heteroaryl groups include, but are not limited to, thienyl, furyl, indolyl, pyrrolyl, pyridinyl, pyrazinyl, oxazolyl, thiaxolyl, quinolinyl, pyrimidinyl, imidazole substituted or unsubstituted triazolyl and substituted or unsubstituted tetrazolyl.

In the case of aryl or heteroaryl which are bicyclic it should be understood that one ring may be aryl while the other is heteroaryl and both being substituted or unsubstituted.

“Hetero atom” means an atom selected from N, O and S.

“Heterocycle” or “heterocyclic ring” means a substituted or unsubstituted 5 to 8 membered, mono- or bicyclic, non-aromatic hydrocarbon, wherein 1 to 3 carbon atoms are replaced by a hetero atom selected from nitrogen, oxygen or sulfur atom. Examples include pyrrolidin-2-yl; pyrrolidin-3-yl; piperidinyl; morpholin-4-yl and the like which in turn can be substituted.

Hydroxy or hydroxyl is a prefix indicating the presence of a monovalent —O—H group.

“Lower” as in “lower alkenyl” means a group having 1 to 6 carbon atoms.

“Nitro” means —NO₂.

Oxo means the group ═O.

Pharmaceutically acceptable,” such as pharmaceutically acceptable carrier, excipient, etc., means pharmacologically acceptable and substantially non-toxic to the subject to which the particular compound is administered.

Pharmaceutically acceptable salt” refers to conventional acid-addition salts or base-addition salts that retain the biological effectiveness and properties of the compounds of the present invention and are formed from suitable non-toxic organic or inorganic acids or organic or inorganic bases. Sample acid-addition salts include those derived from inorganic acids such as hydrochloric acid, hydrobromic acid, hydroiodic acid, sulfuric acid, sulfamic acid, phosphoric acid and nitric acid, and those derived from organic acids such as p-toluenesulfonic acid, salicylic acid, methanesulfonic acid, oxalic acid, succinic acid, citric acid, malic acid, lactic acid, fumaric acid, trifluoro acetic acid and the like. Sample base-addition salts include those derived from ammonium, potassium, sodium and, quaternary ammonium hydroxides, such as for example, tetramethylammonium hydroxide. Chemical modification of a pharmaceutical compound (i.e. drug) into a salt is a technique well known to pharmaceutical chemists to obtain improved physical and chemical stability, hygroscopicity, flowability and solubility of compounds. See, e.g., Ansel et al., Pharmaceutical Dosage Forms and Drug Delivery Systems (1995) at pgs. 456-457.

Substituted,” as in substituted alkyl, means that the substitution can occur at one or more positions and, unless otherwise indicated, that the substituents at each substitution site are independently selected from the specified options. The term “optionally substituted” refers to the fact that one or more hydrogen atoms of a chemical group (with one or more hydrogen atoms) can be, but does not necessarily have to be, substituted with another substituent. In the specification where indicated the various groups may be substituted by preferably, 1-3 substituents independently selected from the group consisting of H, carboxyl, amino, hydroxyl, alkoxy, substituted alkoxy, sulfide, sulfone, sulfonamide, sulfoxide, halogen, nitro, amino, substituted amino, lower alkyl, substituted lower alkyl, lower cycloalkyl, substituted lower cycloalkyl, lower alkenyl, substituted lower alkenyl, lower cycloalkenyl, substituted lower cycloalkenyl, aryl, substituted aryl, heteroaryl, substituted heteroaryl, heterocycle or substituted heterocycle.

Commonly used abbreviations include: acetyl (Ac), aqueous (aq.), atmospheres (Atm), tert-butoxycarbonyl (Boc), di-tert-butyl pyrocarbonate or boc anhydride (BOC₂O), benzyl (Bn), benzotriazol-1-yloxy-tris-(dimethylamino)phosphonium hexafluorophosphate (BOP), butyl (Bu), benzoyl (Bz), Chemical Abstracts Registration Number (CASRN), benzyloxycarbonyl (CBZ or Z), carbonyl diimidazole (CDI), dibenzylideneacetone (DBA), 1,5-diazabicyclo[4.3.0]non-5-ene (DBN), 1,8-diazabicyclo[5.4.0]undec-7-ene (DBU), N,N′-dicyclohexylcarbodiimide (DCC), 1,2-dichloroethane (DCE), dichloromethane (DCM), diethyl azodicarboxylate (DEAD), di-iso-propylazodicarboxylate (DIAD), di-iso-butylaluminumhydride (DIBAL or DIBAL-H), di-iso-propylethylamine (DIPEA), N,N-dimethyl acetamide (DMA), 4-N,N-dimethylaminopyridine (DMAP), N,N-dimethylformamide (DMF), dimethyl sulfoxide (DMSO), 1,1′-bis-(diphenylphosphino)ethane (dppe), 1,1′-bis-(diphenylphosphino)ferrocene (dppf), 1-(3-dimethylaminopropyl)-3-ethylcarbodiimide hydrochloride (EDCI), ethyl (Et), diethyl ether (Et₂O), ethyl acetate (EtOAc), ethanol (EtOH), 2-ethoxy-2H-quinoline-1-carboxylic acid ethyl ester (EEDQ), diethyl ether (Et₂O), O-(7-azabenzotriazole-1-yl)-N,N,N′N′-tetramethyluronium hexafluorophosphate acetic acid (HATU), O-(benzotriazol-1-yl)-N,N,N′,N′-tetramethyluronium hexafluorophosp (HBTU), acetic acid (HOAc), 1-N-hydroxybenzotriazole (HOBt), high pressure liquid chromatography (HPLC), iso-propanol (IPA), lithium hexamethyldisilazide (LiHMDS), lithium diisopropylamide (LDA), methanol (MeOH), melting point (mp), MeSO₂— (mesyl or Ms), methyl (Me), acetonitrile (MeCN), m-chloroperbenzoic acid (MCPBA), mass spectrum (ms), methyl tert-butyl ether (MTBE), N-methylmorpholine (NMM), N-methylpyrrolidone (NMP), pyridinium chlorochromate (PCC), petroleum ether (pet ether, i.e. hydrocarbons), phenyl (Ph), propyl (Pr), iso-propyl (i-Pr), pounds per square inch (psi), bromo-tris-pyrrolidinophosphonium hexafluorophosphate (PyBrOP), pyridine (pyr), room temperature (rt or RT), satd. (saturated), tert-butymethyl ether (TBME), tert-butyldimethylsilyl or t-BuMe₂Si (TBDMS or TBS), triethylamine (TEA or Et₃N), triflate or CF₃SO₂— (Tf), trifluoroacetic acid (TFA), O-benzotriazol-1-yl-N,N,N′,N′-tetramethyluronium tetrafluoroborate (TBTU), thin layer chromatography (TLC), tetrahydrofuran (THF), tetramethylethylenediamine (TMEDA), trimethylsilyl or Me₃Si (TMS), 2-(trimethylsilyl)ethoxymethyl (SEM), p-toluenesulfonic acid monohydrate (TsOH or pTsOH), 4-Me-C₆H₄SO₂— or tosyl (Ts), N-urethane-N-carboxyanhydride (UNCA), 4,5-Bis(diphenylphosphino)-9,9-dimethylxanthene (Xantphos). Conventional nomenclature including the prefixes normal (n), iso (i-), secondary (sec-), tertiary (tert- or -t) and neo- have their customary meaning when used with an alkyl moiety. (J. Rigaudy and D. P. Klesney, Nomenclature in Organic Chemistry, IUPAC 1979 Pergamon Press, Oxford.).

### Compounds and Preparation

Examples of representative compounds within the scope of the invention are provided in the following Table. These examples and preparations which follow are provided to enable those skilled in the art to more clearly understand and to practice the present invention. They should not be considered as limiting the scope of the invention, but merely as being illustrative and representative thereof.

In general, the nomenclature used in this Application is based on AUTONOM™ v.4.0, a Beilstein Institute computerized system for the generation of IUPAC systematic nomenclature. If there is a discrepancy between a depicted structure and a name given that structure, the depicted structure is to be accorded more weight. In addition, if the stereochemistry of a structure or a portion of a structure is not indicated with, for example, bold or dashed lines, the structure or portion of the structure is to be interpreted as encompassing all stereoisomers of it.

TABLE 1

| | | TNKS1¹ | TNKS1² | TNKS2¹ | |
|---|---|---|---|---|---|
| | | IC₅₀ | IC₅₀ | IC₅₀ | |
| | Structure | (μM) | (μM) | (μM) | Name |
| I-1 | 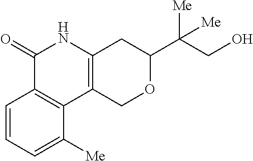 [SMILES: Cc1cccc2c(=O)[nH]c3c(c12)COC(C(C)(C)CO)C3] | 0.0255 | 0.0106 | 0.0284 | 3-(2-hydroxy-1 1- dimethyl-ethyl)-10- methyl-1 3 4 5- tetrahydropyrano[4 3- c]isoquinolin-6-one |
| I-2 | 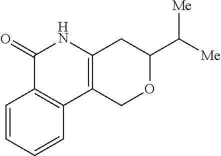 [SMILES: CC(C)C1Cc2[nH]c(=O)c3ccccc3c2CO1] | 0.0372 | | 0.0447 | 3-isopropyl-1 3 4 5- tetrahydropyrano[4 3- c]isoquinolin-6-one |
| I-3 | 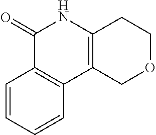 [SMILES: O=c1[nH]c2c(c3ccccc13)COCC2] | 0.421 | | 0.636 | 1 3 4 5- tetrahydropyrano[4 3- c]isoquinolin-6-one |
| I-4 | 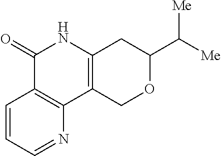 [SMILES: CC(C)C1Cc2[nH]c(=O)c3cccnc3c2CO1] | 0.0609 | | 0.0931 | 8-isopropyl-6 7 8 10- tetrahydropyrano[4 3- h][1 6]naphthyridin-5- one |
| I-5 | 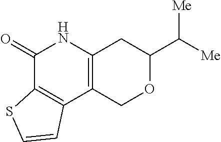 [SMILES: CC(C)C1Cc2[nH]c(=O)c3sccc3c2CO1] | 0.0703 | | 0.0921 | 3-isopropyl-3,4- dihydro-1H- pyrano[4,3- b]thieno[3,2- d]pyridin-6(5H)-one |
| I-6 | 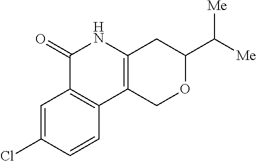 [SMILES: CC(C)C1Cc2[nH]c(=O)c3cc(Cl)ccc3c2CO1] | 0.044 | | 0.0734 | 8-chloro-3-isopropyl- 1 3 4 5- tetrahydropyrano[4 3- c]isoquinolin-6-one |
| I-7 | 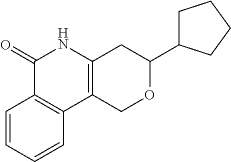 [SMILES: O=c1[nH]c2c(c3ccccc13)COC(C1CCCC1)C2] | 0.0342 | | 0.043 | 3-cyclopentyl-1 3 4 5- tetrahydropyrano[4 3- c]isoquinolin-6-one |
| I-8 | 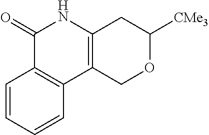 | 0.0259 | | 0.0323 | 3-tert-butyl-1 3 4 5- tetrahydropyrano[4 3- c]isoquinolin-6-one |
| I-9 | 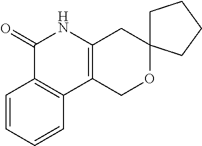 [SMILES: O=c1[nH]c2c(c3ccccc13)COC1(CCCC1)C2] | 0.0729 | | 0.144 | spiro[4 5-dihydro-1H- pyrano[4 3- c]isoquinoline-3 1′- cyclopentane]-6-one |
| I-10 | 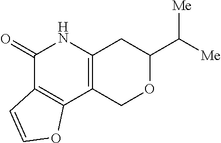 [SMILES: CC(C)C1Cc2[nH]c(=O)c3ccoc3c2CO1] | 0.0937 | | 0.231 | 7-isopropyl-5,6,7,9- tetrahydro-4H- furo[2,3-d]pyrano[4,3- b]pyridin-4-one |
| I-11 | 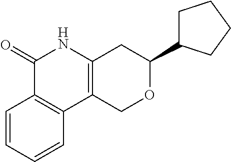 [SMILES: O=c1[nH]c2c(c3ccccc13)CO[C@H](C1CCCC1)C2] | 0.0315 | | 0.102 | (3S)-3-cyclopentyl- 1 3 4 5- tetrahydropyrano[4 3- c]isoquinolin-6-one |
| I-12 | 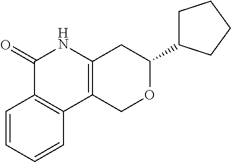 [SMILES: O=c1[nH]c2c(c3ccccc13)CO[C@@H](C1CCCC1)C2] | 0.016 | | 0.0194 | (3R)-3-cyclopentyl- 1 3 4 5- tetrahydropyrano[4 3- c]isoquinolin-6-one |
| I-13 | 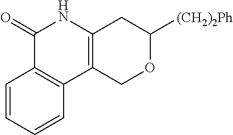 | 0.0408 | | 0.0521 | 3-(2-phenylethyl)- 1 3 4 5- tetrahydropyrano[4 3- c]isoquinolin-6-one |
| I-14 | 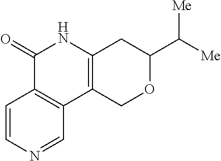 [SMILES: CC(C)C1Cc2[nH]c(=O)c3ccncc3c2CO1] | 0.0972 | | 0.204 | 3-isopropyl-1 3 4 5- tetrahydropyrano[4 3- c][2 6]naphthyridin-6- one |
| I-15 | 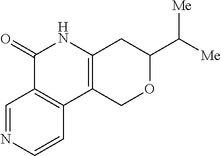 [SMILES: CC(C)C1Cc2[nH]c(=O)c3cnccc3c2CO1] | 0.0508 | | 0.0688 | 3-isopropyl-1 3 4 5- tetrahydropyrano[4 3- c][2 7]naphthyridin-6- one |
| I-16 | 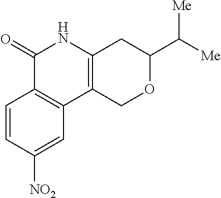 [SMILES: CC(C)C1Cc2[nH]c(=O)c3ccc([N+](=O)[O-])cc3c2CO1] | 0.404 | | 2.7 | 3-isopropyl-9-nitro- 1 3 4 5- tetrahydropyrano[4 3- c]isoquinolin-6-one |
| I-17 | 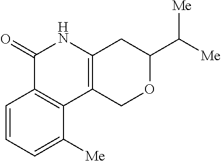 [SMILES: Cc1cccc2c(=O)[nH]c3c(c12)COC(C(C)C)C3] | 0.0319 | | 0.0519 | 3-isopropyl-10- methyl-1 3 4 5- tetrahydropyrano[4 3- c]isoquinolin-6-one |
| I-18 | 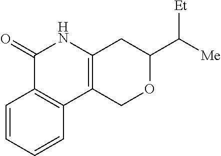 [SMILES: CCC(C)C1Cc2[nH]c(=O)c3ccccc3c2CO1] | 0.0568 | | 0.0883 | 3-sec-butyl-1 3 4 5- tetrahydropyrano[4 3- c]isoquinolin-6-one |
| I-19 | 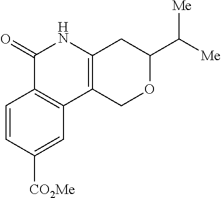 [SMILES: COC(=O)c1ccc2c(=O)[nH]c3c(c2c1)COC(C(C)C)C3] | 0.0841 | | 0.386 | methyl 3-isopropyl-6- oxo-1 3 4 5- tetrahydropyrano[4 3- c]isoquinoline-9- carboxylate |
| I-20 | 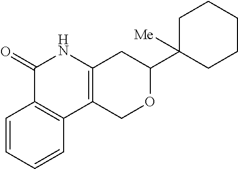 [SMILES: CC1(C2Cc3[nH]c(=O)c4ccccc4c3CO2)CCCCC1] | 0.0255 | | 0.0305 | 3-(1- methylcyclohexyl)- 1 3 4 5- tetrahydropyrano[4 3- c]isoquinolin-6-one |
| I-21 | 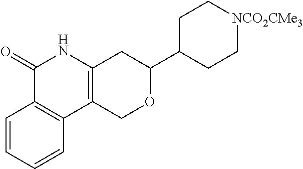 | 0.0335 | | 0.0467 | tert-butyl 4-(6-oxo- 1 3 4 5- tetrahydropyrano[4 3- c]isoquinolin-3- yl)piperidine-1- carboxylate |
| I-22 | 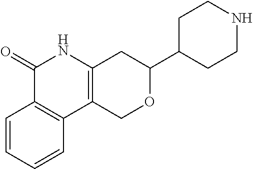 [SMILES: O=c1[nH]c2c(c3ccccc13)COC(C1CCNCC1)C2] | 0.0888 | | 0.109 | 3-(4-piperidyl)-1 3 4 5- tetrahydropyrano [4 3-c]isoquinolin-6-one |
| I-23 | 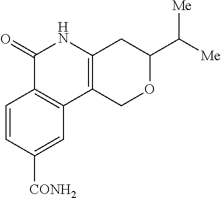 [SMILES: CC(C)C1Cc2[nH]c(=O)c3ccc(C(N)=O)cc3c2CO1] | 0.0886 | | 0.243 | 3-isopropyl-6-oxo- 1 3 4 5- tetrahydropyrano[4 3- c]isoquinoline-9- carboxamide |
| I-24 | 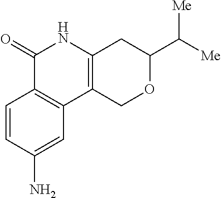 [SMILES: CC(C)C1Cc2[nH]c(=O)c3ccc(N)cc3c2CO1] | 0.473 | | 1.3 | 9-amino-3-isopropyl- 1 3 4 5- tetrahydropyrano[4 3- c]isoquinolin-6-one |
| I-25 | 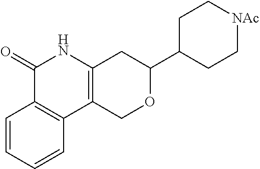 | 0.0255 | | 0.027 | 3-(1-acetyl-4- piperidyl)-1 3 4 5- tetrahydropyrano[4 3- c]isoquinolin-6-one |
| I-26 | 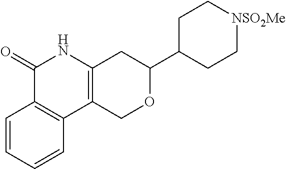 | 0.0482 | | 0.0554 | 3-(1-methylsulfonyl- 4-piperidyl)-1 3 4 5- tetrahydropyrano[4 3- c]isoquinolin-6-one |
| I-27 | 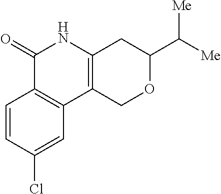 [SMILES: CC(C)C1Cc2[nH]c(=O)c3ccc(Cl)cc3c2CO1] | 0.0391 | | 0.0535 | 9-chloro-3-isopropyl- 1 3 4 5- tetrahydropyrano[4 3- c]isoquinolin-6-one |
| I-28 | 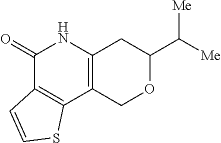 [SMILES: CC(C)C1Cc2[nH]c(=O)c3ccsc3c2CO1] | 0.0818 | | 0.177 | 7-isopropyl-5,6,7,9- tetrahydro-4H- pyrano[4,3- b]thieno[2,3- d]pyridin-4-one |
| I-29 | 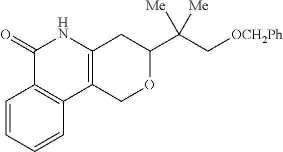 | 0.0672 | | 0.113 | 3-(2-benzyloxy-1 1- dimethyl-ethyl)-1 3 4 5- tetrahydropyrano[4 3- c]isoquinolin-6-one |
| I-30 | 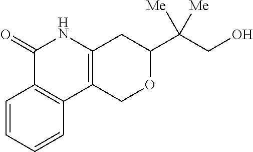 [SMILES: CC(C)(CO)C1Cc2[nH]c(=O)c3ccccc3c2CO1] | 0.0262 | | 0.0334 | 3-(2-hydroxy-1 1- dimethyl-ethyl)- 1 3 4 5-tetrahydropyrano [4 3-c]isoquinolin-6-one |
| I-31 | 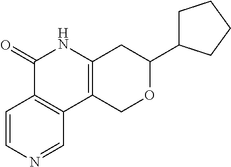 [SMILES: O=c1[nH]c2c(c3cnccc13)COC(C1CCCC1)C2] | 0.0708 | | 0.0891 | 3-cyclopentyl-1 3 4 5- tetrahydropyrano[4 3- c][2 6]naphthyridin-6- one |
| I-32 | 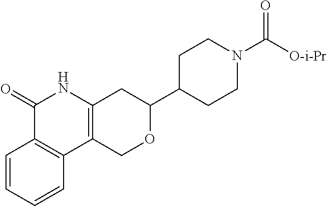 | 0.0308 | | 0.0405 | isopropyl 4-(6-oxo- 1 3 4 5- tetrahydropyrano[4 3- c]isoquinolin-3- yl)piperidine-1- carboxylate |
| I-33 | 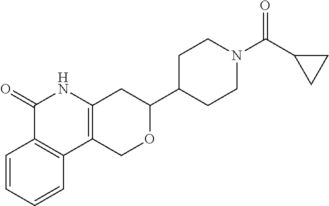 [SMILES: O=C(C1CC1)N1CCC(C2Cc3[nH]c(=O)c4ccccc4c3CO2)CC1] | 0.0365 | | 0.052 | 3-[1- (cyclopropanecarbonyl)- 4-piperidyl]-1 3 4 5- tetrahydropyrano[4 3- c]isoquinolin-6-one |
| I-34 | 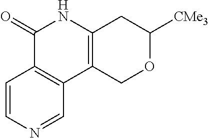 | 0.0849 | | 0.183 | 3-tert-butyl-1 3 4 5- tetrahydropyrano[4 3- c][2 6]naphthyridin-6- one |
| I-35 | 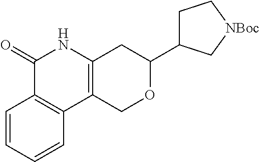 [SMILES: CC(C)(C)OC(=O)N1CCC(C2Cc3[nH]c(=O)c4ccccc4c3CO2)C1] | 0.0379 | | 0.056 | tert-butyl 3-(6-oxo- 1 3 4 5- tetrahydropyrano[4 3- c]isoquinolin-3- yl)pyrrolidine-1- carboxylate |
| I-36 | 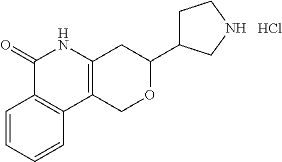 [SMILES: O=c1[nH]c2c(c3ccccc13)COC(C1CCNC1)C2 ; Cl] | 0.168 | | 0.246 | 3-pyrrolidin-3-yl- 1 3 4 5-tetrahydropyrano [4 3-c]isoquinolin-6-one |
| I-37 | 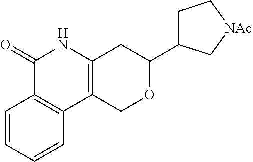 | 0.0499 | | 0.0674 | 3-(1-acetylpyrrolidin- 3-yl)-1 3 4 5- tetrahydropyrano[4 3- c]isoquinolin-6-one |
| I-38 | 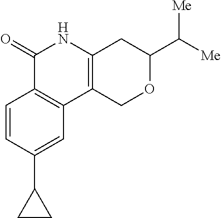 [SMILES: CC(C)C1Cc2[nH]c(=O)c3ccc(C4CC4)cc3c2CO1] | 0.091 | | 0.196 | 9-cyclopropyl-3- isopropyl-1 3 4 5- tetrahydropyrano[4 3- c]isoquinolin-6-one |
| I-39 | 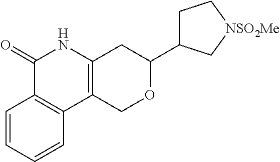 | 0.039 | | 0.0389 | 3-(1- methylsulfonyl- pyrrolidin-3-yl)- 1 3 4 5- tetrahydropyrano[4 3- c]isoquinolin-6-one |
| I-40 | 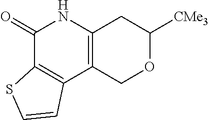 | 0.0326 | | 0.0355 | 3-(tert-butyl)-3,4- dihydro-1H- pyrano[4,3- b]thieno[3,2- d]pyridin-6(5H)-one |
| I-41 | 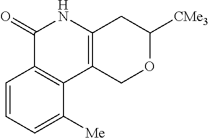 | 0.0212 | | 0.026 | 3-tert-butyl-10- methyl-1 3 4 5- tetrahydropyrano[4 3- c]isoquinolin-6-one |
| I-42 | 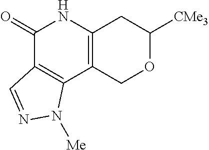 | 0.0609 | | 0.0877 | 7-(tert-butyl)-1- methyl-5,6,7,9- tetrahydropyrano[4,3- b]pyrazolo[3,4- d]pyridin-4(1H)-one |
| I-43 | 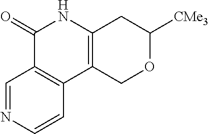 | 0.0439 | | 0.049 | 3-tert-butyl-1 3 4 5- tetrahydropyrano[4 3- c][2 7]naphthyridin-6- one |
| I-44 | 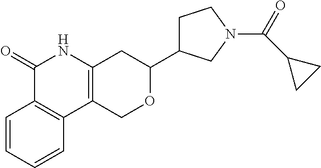 [SMILES: O=C(C1CC1)N1CCC(C2Cc3[nH]c(=O)c4ccccc4c3CO2)C1] | 0.0422 | | 0.0656 | 3-[1- (cyclopropane- carbonyl)pyrrolidin-3- yl]-1 3 4 5- tetrahydropyrano[4 3- c]isoquinolin-6-one |
| I-45 | 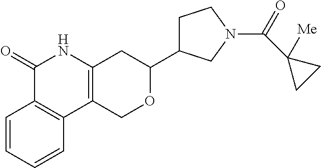 [SMILES: CC1(C(=O)N2CCC(C3Cc4[nH]c(=O)c5ccccc5c4CO3)C2)CC1] | 0.0647 | | 0.0867 | 3-[1-(1- methylcyclopropane- carbonyl)pyrrolidin-3- yl]-1 3 4 5- tetrahydropyrano[4 3- c]isoquinolin-6-one |
| I-46 | 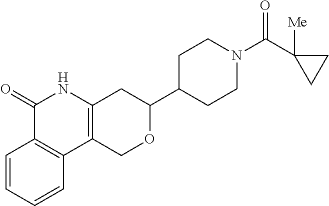 [SMILES: CC1(C(=O)N2CCC(C3Cc4[nH]c(=O)c5ccccc5c4CO3)CC2)CC1] | 0.0332 | | 0.0443 | 3-[1-(1- methylcyclopropane- carbonyl)-4-piperidyl]- 1 3 4 5- tetrahydropyrano[4 3- c]isoquinolin-6-one |
| I-47 | 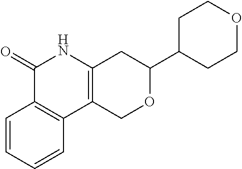 [SMILES: O=c1[nH]c2c(c3ccccc13)COC(C1CCOCC1)C2] | 0.0324 | | 0.0368 | 3-tetrahydropyran-4- yl-1 3 4 5- tetrahydropyrano[4 3- c]isoquinolin-6-one |
| I-48 | 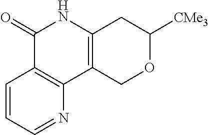 | 0.0346 | | 0.0554 | 8-tert-butyl-6 7 8 10- tetrahydropyrano[4 3- h][l 6]naphthyridin-5- one |
| I-49 | 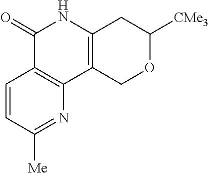 | 0.514 | | 0.0775 | 8-tert-butyl-2-methyl- 6 7 8 10- tetrahydropyrano[4 3- h][l 6]naphthyridin-5- one |
| I-50 | 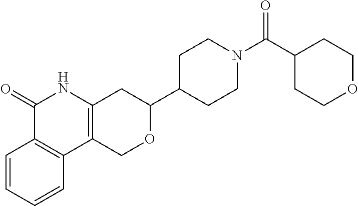 [SMILES: O=C(C1CCOCC1)N1CCC(C2Cc3[nH]c(=O)c4ccccc4c3CO2)CC1] | 0.0356 | | 0.0442 | 3-[1-(tetrahydropyran- 4-carbonyl)-4- piperidyl]-1 3 4 5- tetrahydropyrano[4 3- c]isoquinolin-6-one |
| I-51 | 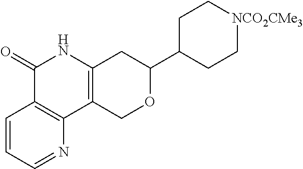 | 0.039 | | 0.0498 | tert-butyl 4-(5-oxo- 6 7 8 10- tetrahydropyrano[4 3- h][l 6]naphthyridin-8- yl)piperidine-1- carboxylate |
| I-52 | 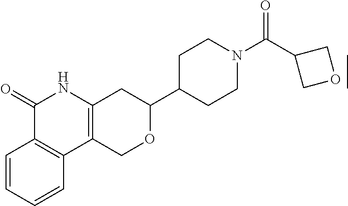 [SMILES: O=C(C1COC1)N1CCC(C2Cc3[nH]c(=O)c4ccccc4c3CO2)CC1] | 0.0273 | | 0.0317 | 3-[1-(oxetane-3- carbonyl)-4- piperidyl]-1 3 4 5- tetrahydropyrano[4 3- c]isoquinolin-6-one |
| I-53 | 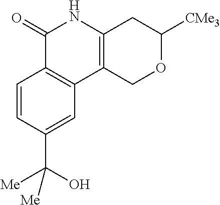 | 1.1 | | 13.9 | 3-tert-butyl-9-(1- hydroxy-1-methyl- ethyl)-1 3 4 5- tetrahydropyrano[4 3- c]isoquinolin-6-one |
| I-54 | 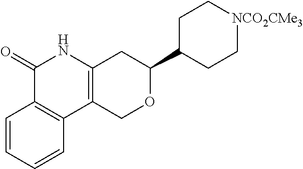 | 0.0214 | | 0.0257 | tert-butyl 4-[(3S)-6- oxo-1 3 4 5- tetrahydropyrano[4 3- c]isoquinolin-3- yl]piperidine-1- carboxylate |
| I-55 | 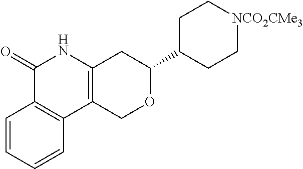 | 0.0432 | | 0.0992 | tert-butyl 4-[(3R)-6- oxo-1 3 4 5- tetrahydropyrano[4 3- c]isoquinolin-3- yl]piperidine-1- carboxylate |
| I-56 | 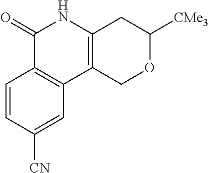 | 0.133 | | 1.5 | 3-tert-butyl-6-oxo- 1 3 4 5- tetrahydropyrano[4 3- c]isoquinoline-9- carbonitrile |
| I-57 | 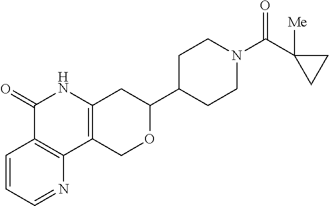 [SMILES: CC1(C(=O)N2CCC(C3Cc4[nH]c(=O)c5cccnc5c4CO3)CC2)CC1] | 0.0402 | | 0.0455 | 8-[1-(1- methylcyclopropane- carbonyl)-4-piperidyl]- 6 7 8 10- tetrahydropyrano[4 3- h][1 6]naphthyridin-5- one |
| I-58 | 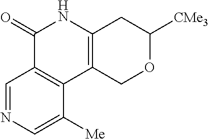 | 0.0259 | | 0.0257 | 3-tert-butyl-10- methyl-1 3 4 5- tetrahydropyrano[4 3- c][2 7]naphthyridin-6- one |
| I-59 | 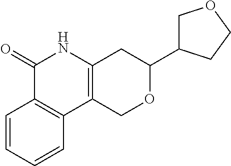 [SMILES: O=c1[nH]c2c(c3ccccc13)COC(C1CCOC1)C2] | 0.0336 | | 0.0344 | 3-tetrahydrofuran-3- yl-1 3 4 5- tetrahydropyrano[4 3- c]isoquinolin-6-one |
| I-60 | 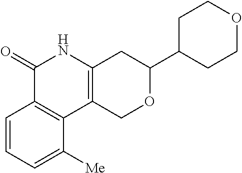 [SMILES: Cc1cccc2c(=O)[nH]c3c(c12)COC(C1CCOCC1)C3] | 0.0241 | | 0.0179 | 10-methyl-3- tetrahydropyran-4-yl- 1 3 4 5- tetrahydropyrano[4 3- c]isoquinolin-6-one |
| I-61 | 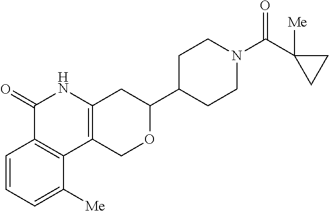 [SMILES: Cc1cccc2c(=O)[nH]c3c(c12)COC(C1CCN(C(=O)C2(C)CC2)CC1)C3] | 0.0175 | | 0.021 | 10-methyl-3-[1-(1- methylcyclopropane- carbonyl)-4-piperidyl]- 1 3 4 5- tetrahydropyrano[4 3- c]isoquinolin-6-one |
| I-62 | 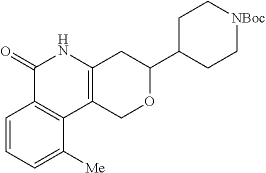 [SMILES: Cc1cccc2c(=O)[nH]c3c(c12)COC(C1CCN(C(=O)OC(C)(C)C)CC1)C3] | 0.0265 | | 0.0237 | tert-butyl 4-(10- methyl-6-oxo-1 3 4 5- tetrahydropyrano[4 3- c]isoquinolin-3- yl)piperidine-1- carboxylate |
| I-63 | 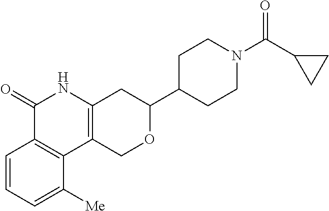 [SMILES: Cc1cccc2c(=O)[nH]c3c(c12)COC(C1CCN(C(=O)C2CC2)CC1)C3] | 0.0283 | | 0.0269 | 3-[1- (cyclopropanecarbonyl)- 4-piperidyl]-10- methyl-1 3 4 5- tetrahydropyrano[4 3- c]isoquinolin-6-one |
| I-64 | 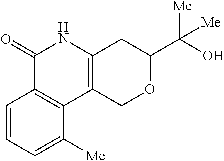 [SMILES: Cc1cccc2c(=O)[nH]c3c(c12)COC(C(C)(C)O)C3] | 0.032 | | 0.0367 | 3-(1-hydroxy-1- methyl-ethyl)-10- methyl-1 3 4 5- tetrahydropyrano[4 3- c]isoquinolin-6-one |
| I-65 | 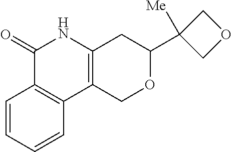 [SMILES: CC1(C2Cc3[nH]c(=O)c4ccccc4c3CO2)COC1] | 0.0814 | | 0.101 | 3-(3-methyloxetan-3- yl)-1 3 4 5- tetrahydropyrano[4 3- c]isoquinolin-6-one |
| I-66 | 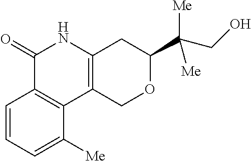 [SMILES: Cc1cccc2c(=O)[nH]c3c(c12)CO[C@H](C(C)(C)CO)C3] | 0.0261 | | 0.0107 | (3S)-3-(2-hydroxy- 1 1-dimethyl-ethyl)-10- methyl-1 3 4 5- tetrahydropyrano[4 3- c]isoquinolin-6-one |
| I-67 | 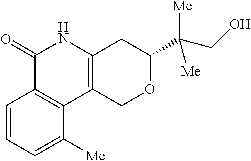 [SMILES: Cc1cccc2c(=O)[nH]c3c(c12)CO[C@@H](C(C)(C)CO)C3] | 0.0276 | | 0.0214 | (3R)-3-(2-hydroxy- 1 1-dimethyl-ethyl)-10- methyl-1 3 4 5- tetrahydropyrano[4 3- c]isoquinolin-6-one |
| I-68 | 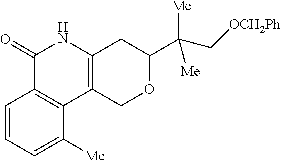 | 0.028 | | 0.0287 | 3-(2-benzyloxy-1 1- dimethyl-ethyl)-10- methyl-1 3 4 5- tetrahydropyrano[4 3- c]isoquinolin-6-one |
| I-69 | 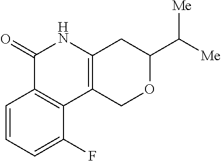 [SMILES: CC(C)C1Cc2[nH]c(=O)c3cccc(F)c3c2CO1] | | 0.0112 | | 10-fluoro-3-isopropyl- 1 3 4 5- tetrahydropyrano[4 3- c]isoquinolin-6-one |
| I-70 | 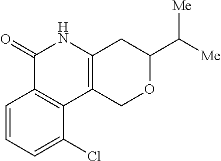 [SMILES: CC(C)C1Cc2[nH]c(=O)c3cccc(Cl)c3c2CO1] | | 0.0129 | | 10-chloro-3-isopropyl- 1 3 4 5- tetrahydropyrano[4 3- c]isoquinolin-6-one |
| I-71 | 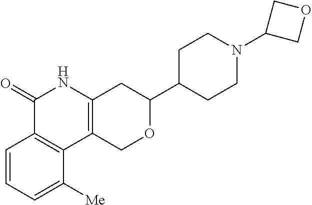 [SMILES: Cc1cccc2c(=O)[nH]c3c(c12)COC(C1CCN(C2COC2)CC1)C3] | | 0.00201 | | 10-methyl-3-[1- (oxetan-3-yl)-4- piperidyl]-1 3 4 5- tetrahydropyrano[4 3- c]isoquinolin-6-one |
| I-72 | 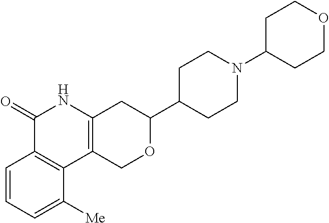 [SMILES: Cc1cccc2c(=O)[nH]c3c(c12)COC(C1CCN(C2CCOCC2)CC1)C3] | | 0.0924 | | 10-methyl-3-(1- tetrahydropyran-4-yl- 4-piperidyl)-1 3 4 5- tetrahydropyrano[4 3- c]isoquinolin-6-one |
| I-73 | 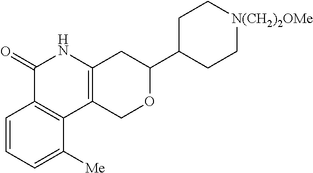 | | 0.0294 | | 3-[1-(2- methoxyethyl)-4- piperidyl]-10-methyl- 1 3 4 5- tetrahydropyrano[4 3- c]isoquinolin-6-one |
| I-74 | 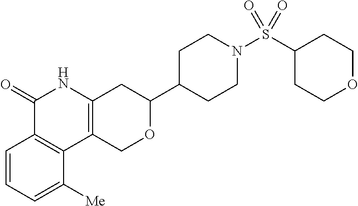 [SMILES: Cc1cccc2c(=O)[nH]c3c(c12)COC(C1CCN(S(=O)(=O)C2CCOCC2)CC1)C3] | | 0.0095 | | 10-methyl-3-(1- tetrahydropyran-4- ylsulfonyl-4- piperidyl)-1 3 4 5- tetrahydropyrano(4 3- c]isoquinolin-6-one |
| I-75 | 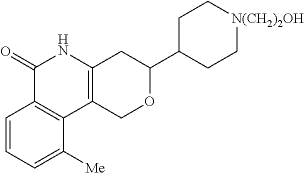 | | 0.0272 | | 3-[1-(2-hydroxyethyl)- 4-piperidyl]-10- methyl-1 3 4 5- tetrahydropyrano[4 3- c]isoquinolin-6-one |
| I-76 | 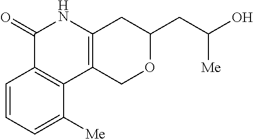 [SMILES: Cc1cccc2c(=O)[nH]c3c(c12)COC(CC(C)O)C3] | | 0.0159 | | 3-(2-hydroxypropyl)- 10-methyl-1 3 4 5- tetrahydropyrano[4 3- c]isoquinolin-6-one |

¹Tankyrase 1 and 2 inhibition assay in Example 71
²Tankyrase 1 inhibition assay in Example 72

Compounds of the invention can be made by a variety of methods depicted in the illustrative synthetic reactions described below in the Examples section.

The starting materials and reagents used in preparing these compounds generally are either available from commercial suppliers, such as Aldrich Chemical Co., or are prepared by methods known to those skilled in the art following procedures set forth in references such as Fieser and Fieser's Reagents for Organic Synthesis; Wiley & Sons: New York, 1991, Volumes 1-15; Rodd's Chemistry of Carbon Compounds, Elsevier Science Publishers, 1989, Volumes 1-5 and Supplementals; and Organic Reactions, Wiley & Sons: New York, 1991, Volumes 1-40. It should be appreciated that the synthetic reaction schemes shown in the Examples section are merely illustrative of some methods by which the compounds of the invention can be synthesized, and various modifications to these synthetic reaction schemes can be made and will be suggested to one skilled in the art having referred to the disclosure contained in this application.

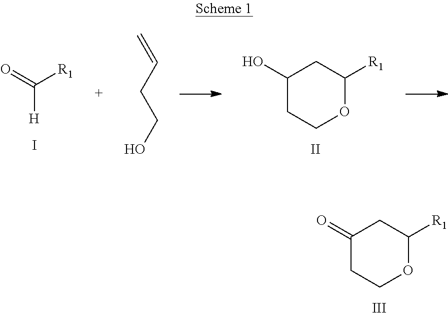

The compounds of formula I, in Scheme 1, where R₁ is hydrogen, alkyl, heterocycloalkyl or substituted heterocycloalkyl, can be purchased from commercial sources or prepared as described below.

The compounds of formula II where R₁ is hydrogen, alkyl, heterocycloalkyl or substituted heterocycloalkyl can be prepared using a Prins reaction by reacting the appropriate heterocyclic starting material with commercially available but-3-en-1-ol under acidic conditions, preferably trifluoroacetic acid in methylene chloride (see e.g., Hanschke, E., Chem. Ber. 1955 88:1053; Barry, C. S., Bushby, N., Harding, J. R., Hughes, R. A., Parker, G. D., Roe, R., Willis, C. L., Chem. Commun. 2005 3727; and Cornelius, N. and Frater, G., Helvetica Chimica Acta 1987 70:396) followed by basic hydrolysis, preferably sodium carbonate in methanol.

The compounds of formula III where R₁ is hydrogen, alkyl, heterocycloalkyl or substituted heterocycloalkyl can be prepared by reacting the appropriately substituted II with an oxidizing reagent, such as PPC and silica gel (see e.g., Anzalone, L. and Hirsch, J. A., J. Org. Chem., 1985 50:2607-2613 and Haslegrave, J. A. and Jones, J. B., J. Amer. Chem. Soc. 1982 104:4666-4671) in an appropriate solvent, such as methylene chloride.

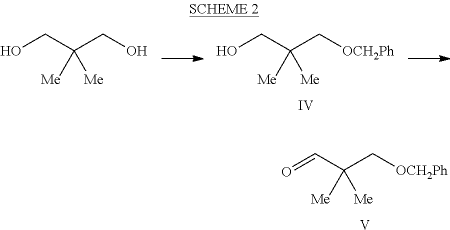

The compound of formula IV, in Scheme 2, can be prepared by reacting 2,2-dimethyl-propane-1,3-diol with an appropriate base, such as sodium t-butoxide, and a benzyl halide, such as chloride or bromide in an appropriate solvent, preferably dioxane (Kalesse, Markus; Quitschalle, Monika; Claus, Eckhard; Gerlach, Kai; Pahl, Axel; Meyer, Hartmut H., Eur. J. Org. Chem. 1999 2817-2824).

The compound of formula V can be prepared by reacting IV with an oxidizing reagent, such as PPC and silica gel (see e.g., Anzalone, L. and Hirsch, J. A., J. Org. Chem., 1985 50:2607-2613 and Haslegrave, J. A. and Jones, J. B., J. Amer. Chem. Soc. 1982 104:4666-4671) in an appropriate solvent, such as methylene chloride.

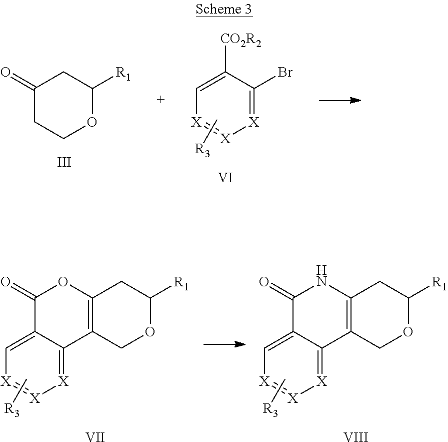

The compounds of formula VII, in Scheme 3, where R₁ is hydrogen, alkyl, heterocycloalkyl or substituted heterocycloalkyl, R₂ is lower alkyl, R₃ is hydrogen, halogen, lower alkyl, lower cycloalkyl, nitro, or carboxymethyl, and X is CH or N, can be prepared from compounds of formula III where R₁ is hydrogen, alkyl, heterocycloalkyl or substituted heterocycloalkyl and compounds of the formula VI where R₁ is hydrogen, alkyl, heterocycloalkyl or substituted heterocycloalkyl, R₃ is hydrogen, halogen, lower alkyl, lower cycloalkyl, nitro, or carboxymethyl, and X is CH or N by metal catalyzed cross coupling with an appropriate catalyst, preferably a base, such as cesium carbonate, and ligand, such as XantPhos in an appropriate solvent such as dioxane and heating either in an oil bath or by microwave (see e.g., Willis, M. C., Taylor, D. and Gillmore A. T., Org. Lett. 2004 6:4755-4757, Konno, F., Ishikawa, T., Kawahata, M. and Yamaguchi, K., J. Org. Chem. 2006 71:9818-9823 and Tadd, A. C., Fielding, M. R. and Willis, M. C., Chem. Commun. 2009 6744-6746).

The compounds of formula VIII where R₁ is hydrogen, alkyl, heterocycloalkyl or substituted heterocycloalkyl, R₃ is hydrogen, halogen, lower alkyl, lower cycloalkyl, nitro, or carboxymethyl, and X is CH or N, can be prepared from compounds of formula VII by heating with ammonia in an appropriate solvent, such as methanol (see e.g., Yamamoto, M., Hashigaki, K., Iwahashi, H., Ninomiya, M., Yakugaku Zasshi 1978 98:1498, Ferrer, S., Naughton, D. P., Parveen, I., Threadgill, M. D. J. Chem. Soc. Perkin 1 2002 335 and Kozikowski, A. P., Reddy, E. R., Miller, C. P., J. Chem. Soc. Perkin 1 1990 195). The R₁ heterocycloalkyl derivatives may be in a protected form that may be deprotected at some point in the synthesis. The R₃ derivatives could also be further transformed through standard chemical manipulation.

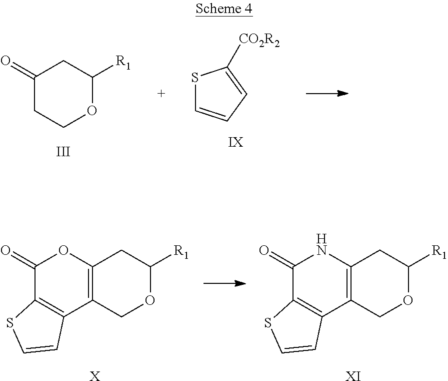

The compounds of formula X, in Scheme 4, where R₁ is hydrogen, alkyl, heterocycloalkyl or substituted heterocycloalkyl, can be prepared from compounds of formula III where R₁ is hydrogen, alkyl, heterocycloalkyl or substituted heterocycloalkyl and compounds of the formula IX where R₂ is lower alkyl, by metal catalyzed cross coupling with an appropriate catalyst, preferably Pd₂(dba)₃, a base, such as cesium carbonate, and ligand, such as XantPhos in an appropriate solvent such as dioxane and heating either in an oil bath or by microwave (see e.g., Willis, M. C., Taylor, D. and Gillmore A. T., Org. Lett. 2004 6:4755-4757; Konno, F., Ishikawa, T., Kawahata, M. and Yamaguchi, K., J. Org. Chem. 2006 71:9818-9823; and Tadd, A. C., Fielding, M. R. and Willis, M. C., Chem. Commun. 2009 6744-6746).

The compounds of formula XI where R₁ is hydrogen, alkyl, heterocycloalkyl or substituted heterocycloalkyl, can be prepared from compounds of formula X by heating with ammonia in an appropriate solvent, such as methanol (see e.g., Yamamoto, M., Hashigaki, K., Iwahashi, H., Ninomiya, M., Yakugaku Zasshi 1978 98:1498, Ferrer, S., Naughton, D. P., Parveen, I., Threadgill, M. D. J. Chem. Soc. Perkin 1 2002 335 and Kozikowski, A. P., Reddy, E. R., Miller, C. P., J. Chem. Soc. Perkin 1 1990 195)

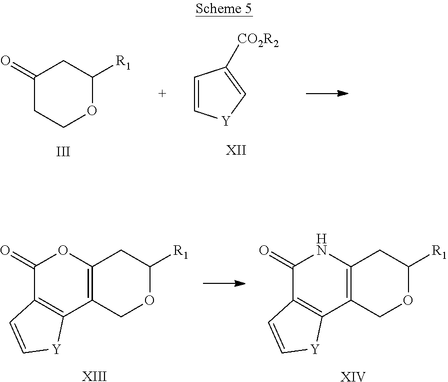

The compounds of formula XIII, in Scheme 5, where R₁ is hydrogen, alkyl, heterocycloalkyl or substituted heterocycloalkyl and Y is oxygen or sulfur, can be prepared from compounds of formula III where R₁ is hydrogen, alkyl, heterocycloalkyl or substituted heterocycloalkyl and compounds of the formula XII where R₂ is lower alkyl, by metal catalyzed cross coupling with an appropriate catalyst, preferably Pd₂(dba)₃, a base, such as cesium carbonate, and ligand, such as XantPhos in an appropriate solvent such as dioxane and heating either in an oil bath or by microwave (see e.g., Willis, M. C., Taylor, D. and Gillmore A. T., Org. Lett. 2004 6:4755-4757, Konno, F., Ishikawa, T., Kawahata, M. and Yamaguchi, K., J. Org. Chem. 2006 71:9818-9823 and Tadd, A. C., Fielding, M. R. and Willis, M. C., Chem. Commun. 2009 6744-6746).

The compounds of formula XIV where R₁ is hydrogen, alkyl, heterocycloalkyl or substituted heterocycloalkyl and Y is oxygen or sulfur, can be prepared from compounds of formula XIII by heating with ammonia in an appropriate solvent, such as methanol (see e.g., Yamamoto, M., Hashigaki, K., Iwahashi, H., Ninomiya, M., Yakugaku Zasshi 1978 98:1498, Ferrer, S., Naughton, D. P., Parveen, I., Threadgill, M. D. J. Chem. Soc. Perkin 1 2002 335 and Kozikowski, A. P., Reddy, E. R., Miller, C. P., J. Chem. Soc. Perkin 1 1990 195).

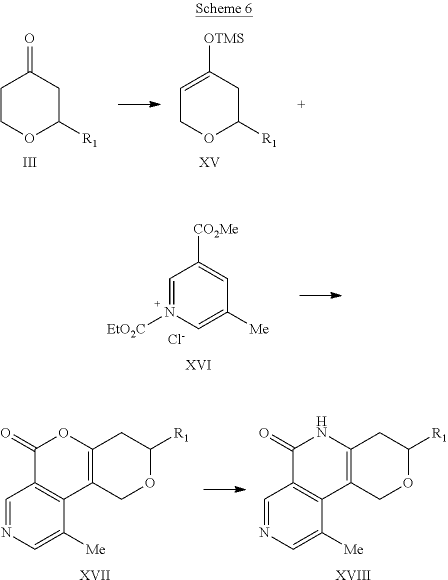

Compounds of the formula XV, in Scheme 6, where R₁ is hydrogen, alkyl, heterocycloalkyl or substituted heterocycloalkyl, can be prepared from compounds of the formula III, where R₁ is hydrogen, alkyl, heterocycloalkyl or substituted heterocycloalkyl, by treatment with lithium diisopropylamine and chlorotrimethylsilane in an appropriate solvent, such as tetrahydrofuran, with cooling (see e.g., Zimmerman, H. E. and Nesterov, E. E., J. Am. Chem. Soc. 2003 125:5422; Denmark, S. E., Dappen, M. S., Sear, N. L., Jacobs, R. T. J. Am. Chem. Soc. 1990 112:3466). Compounds of formula XVII, where R₁ is hydrogen, alkyl, heterocycloalkyl or substituted heterocycloalkyl, can be prepared from compounds of formula XV and XVI, prepared from 5-methyl-nicotinic acid and ethyl chloroformate, in an appropriate solvent, such as methylene chloride (see e.g., Wada, M., Nishihara, Y., Akiba, K. Tetrahedron Lett. 1985 26:3267, Akiba, K., Nishihara, Y., Wada, M., Tetrahedron Lett. 1983 24:5269; and Comins, D. L. and Brown, J. D., Tetrahedron Lett. 1984 25:3297).

The compounds of formula XVIII where R₁ is hydrogen, alkyl, heterocycloalkyl or substituted heterocycloalkyl, can be prepared from compounds of formula XVII by heating with ammonia in an appropriate solvent, such as methanol (see e.g., Yamamoto, M., Hashigaki, K., Iwahashi, H., Ninomiya, M., Yakugaku Zasshi 1978 98:1498, Ferrer, S., Naughton, D. P., Parveen, I., Threadgill, M. D. J. Chem. Soc. Perkin 1 2002 335 and Kozikowski, A. P., Reddy, E. R., Miller, C. P., J. Chem. Soc. Perkin 1 1990 195).

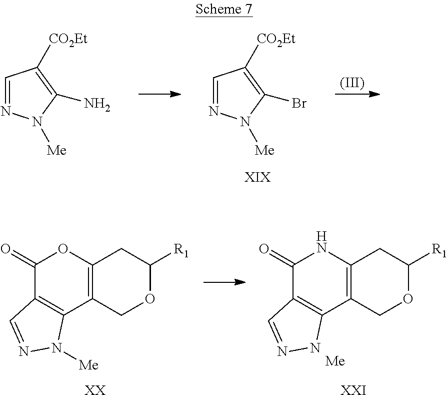

Compound XIX, in Scheme 7, can be prepared from 5-amino-1-methyl-1H-pyrazole-4-carboxylic acid ethyl ester with tert-butyl nitrite and copper (II) bromide in an appropriate solvent such as acetonitrile with heating (see for example, Gillespie, P., Goodnow, R. A., Zhang, Q., US2006/0223852 A1, 2006).

Compounds of formula XX, where R₁ is hydrogen, alkyl, heterocycloalkyl or substituted heterocycloalkyl, can be prepared from compounds of formula III where R₁ is hydrogen, alkyl, heterocycloalkyl or substituted heterocycloalkyl and compound XIX by metal catalyzed cross coupling with an appropriate catalyst, preferably Pd₂(dba)₃, a base, such as cesium carbonate, and ligand, such as XantPhos in an appropriate solvent such as dioxane and heating either in an oil bath or by microwave (see e.g., Willis, M. C., Taylor, D. and Gillmore A. T., Org. Lett. 2004 6:4755-4757, Konno, F., Ishikawa, T., Kawahata, M. and Yamaguchi, K., J. Org. Chem. 2006 71:9818-9823 and Tadd, A. C., Fielding, M. R. and Willis, M. C., Chem. Commun. 2009 6744-6746.

The compounds of formula XXI where R₁ is hydrogen, alkyl, heterocycloalkyl or substituted heterocycloalkyl, can be prepared from compounds of formula XX by heating with ammonia in an appropriate solvent, such as methanol.

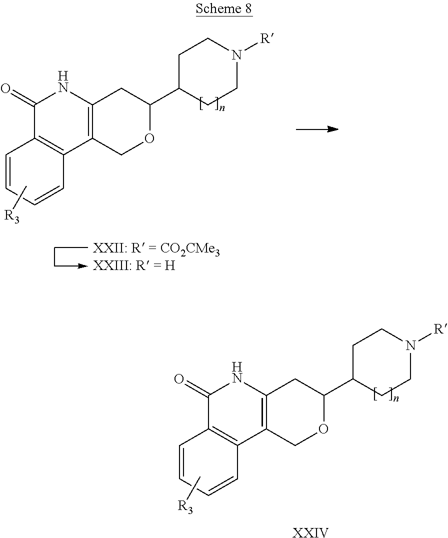

Compounds of formula XXIII, in Scheme 8, where R₃ is hydrogen, halogen, lower alkyl, lower cycloalkyl, nitro, or carboxymethyl and n=0 or 1, can be prepared from compounds of the formula XXII by treatment with and acid, preferably trifluoroacetic acid, in and appropriate solvent, such as methylene chloride. Salts of compounds of formula XXIII can be prepared by counterion exchange through standard chemical manipulation.

Compounds of formula XXIV, where R₃ is hydrogen, halogen, lower alkyl, lower cycloalkyl, nitro, or carboxymethyl, R₄ is lower alkyl carbonyl, lower cycloalkyl carbonate, heterocycloalkyl carbonyl or lower alkyl sulfonyl and n=0 or 1, can be prepared by treatment with lower alkyl chloroformates, lower alkyl acids and an appropriate condensation reagent, such as EDC or HBTU, lower alkyl sulfonyl chlorides, heterocycloalkyl carbonyl chlorides or heterocycloalkyl acids and an appropriate condensation reagent, such as ECD or HBTU, in an appropriate solvent with an appropriate base through standard chemical manipulation.

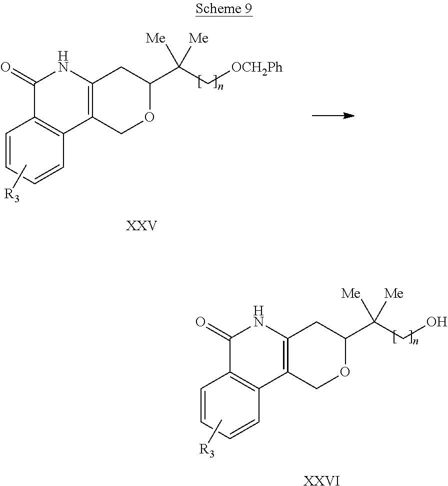

The compounds of formula XXVI, in Scheme 9, where R₃ is hydrogen, halogen, lower alkyl, lower cycloalkyl, nitro, or carboxymethyl, can be prepared from compounds of formula XXV by standard metal catalyzed hydrogenation using a catalyst, preferably Pd(OH)₂ in an appropriate solvent, with a trace of acid, under hydrogen pressure (see e.g., Suenaga, K., Hoshino, H., Yoshii, T, Mori, K., Sone, H., Bessho, Y., Sakakura, A., Hayakawa, I., Ymamda, K., Kigoshi, H., Tetrahedron 2006 62:7687).

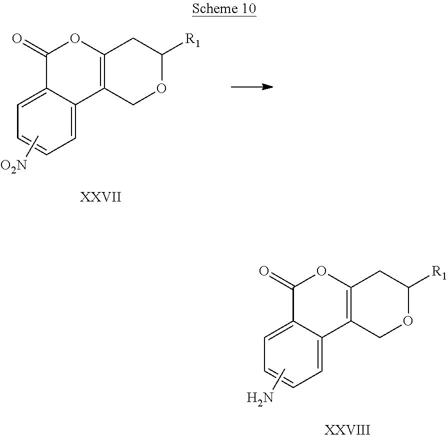

The compounds of formula XXVIII, in Scheme 10, where R₁ is hydrogen, alkyl, heterocycloalkyl or substituted heterocycloalkyl can be prepared by treatment of compounds XXVII in an appropriate solvent, such as ethanol and water, with ammonium chloride and iron and heating (see for example, Yah, S., Appleby, T., Gunic, E., Shim, J. H., Tasu, T., Kim, H., Rong, F., Chen, H., Hamatake, R., Wu, J. Z., Hong, Z, Yao, N., Bioorg. Med. Chem. Lett. 2007 17:28).

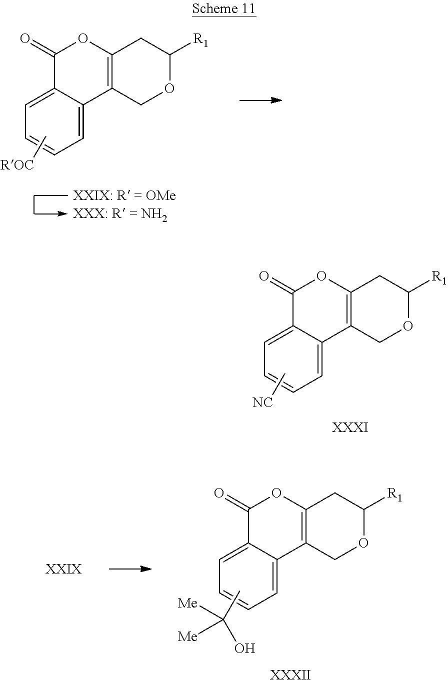

Compounds of formula XXX, in Scheme 11, where R₁ is hydrogen, alkyl, heterocycloalkyl or substituted heterocycloalkyl can be prepared by treatment of compounds XXIX as in (Liu, R., Arrington, M. H., Hopper, A., Tehim, A., WO 2006/071988 A1).

Compounds of formula XXXI, where R₁ is hydrogen, alkyl, heterocycloalkyl or substituted heterocycloalkyl can be prepared by treatment of compounds XXX by treatment with trifluoroacetic anhydride in an appropriate solvent, such as N-methylpyrrolidinone, with an appropriate base, such as triethylamine (see e.g., Yen, C-F, Huang, C-P, Hu, C-K, Chou, M-C, King, C-H. R., US 2008/0242861).

Compounds of formula XXXII, where R₁ is hydrogen, alkyl, heterocycloalkyl or substituted heterocycloalkyl can be prepared by treatment of compounds XXIX, with methylmagnesium bromide in an appropriate solvent, such as tetrahydrofuran (see for example, Galaud, F. and Lubell, W. D., Peptide Science 2005 80:665 and Machacek, M. R., Haidle, A., Zabierek, A. A., Konrad, K. M., Altman, M. D., WO 2010/011375 A2).

The following preparations and examples are given to enable those skilled in the art to more clearly understand and to practice the present invention. They should not be considered as limiting the scope of the invention, but merely as being illustrative and representative thereof.

The starting materials and the intermediates of the synthetic reaction schemes can be isolated and purified if desired using conventional techniques, including but not limited to, filtration, distillation, crystallization, chromatography, and the like. Such materials can be characterized using conventional means, including physical constants and spectral data.

Unless specified to the contrary, the reactions described herein are typically conducted under an inert atmosphere at atmospheric pressure at a reaction temperature range of from about −78° C. to about 150° C., often from about 0° C. to about 125° C., and more often and conveniently at about room (or ambient) temperature, e.g., about 20° C.

Preparative reverse-phase high-pressure liquid chromatography (RP HPLC) was performed using one of the following systems: (A) a Waters Delta prep 4000 pump/controller, a 486 detector set at 215 nm, and a LIMB Ultrorac fraction collector; or (B) a Sciex LC/MS system with a 150 EX single quad mass spec, a Shimadzu LC system, a LEAP autoinjector, and a Gilson fraction collector. The sample was dissolved in a mixture of acetonitrile/20 mM aqueous ammonium acetate or acetonitrile/water/TFA, applied on a Pursuit C-18 20×100 mm column and eluted at 20 mL/min with a linear gradient of 10%-90% B, where (A): 20 mM aqueous ammonium acetate (pH 7.0) and (B): acetonitrile or (A): water with 0.05% TFA and (B): acetonitrile with 0.05% TFA.

Flash chromatography was performed using standard silica gel chromatography, pre-packed silica columns (Analogix or Single Step) with an Analogix BSR pump system, AnaLogix IntelliFlash Automated, or Teledyne-Isco CombiFlash Companion systems. Reactions heated in a microwave were performed using the Biotage Initiator 60 microwave or the CEM Explore microwave.

### Intermediate A

### 2-Isopropyl-tetrahydro-pyran-4-ol

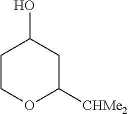

A solution of but-3-en-1-ol (1.0 g, 13.9 mmol) and isobutyraldehyde (2.00 g, 27.7 mmol) in methylene chloride (62 mL) was cooled to 0° C. Trifluoroacetic acid (25 mL) was added slowly under nitrogen atmosphere. The reaction mixture was allowed to warm to room temperature and stirred for 18 h. The resulting mixture was carefully washed with a saturated sodium bicarbonate solution until the aqueous layer remained basic. The organic layer was concentrated in vacuo and the residue was taken up with methanol (100 mL). The reaction mixture was treated with potassium carbonate (5 g, 13.9 mmol) and allowed to stir for 30 min. The resulting mixture was filtered and concentrated in vacuo. The residue was diluted with water and extracted with methylene chloride. The combined organic layer was dried over sodium sulfate, filtered, and concentrated in vacuo to give a crude oil (1.37 g, 68%) which was taken on without further purification. ¹H NMR (CDCl₃) δ: 4.02 (ddd, J=11.7, 4.9, 1.5 Hz, 1H), 3.76 (tt, J=11.0, 4.7 Hz, 1H), 3.37 (td, J=12.1, 1.9 Hz, 1H), 2.98 (ddd, J=11.2, 6.1, 1.9 Hz, 1H), 1.96 (ddt, J=12.1, 4.4, 2.1 Hz, 1H), 1.88 (ddq, J=12.3, 4.4, 2.1 Hz, 1H), 1.71 (sxt, J=6.8 Hz, 1H), 1.38-1.56 (m, 1H), 1.19 (q, J=11.5 Hz, 1H), 0.94 (d, J=6.8 Hz, 3H), 0.91 (d, J=6.8 Hz, 3H)

In an analogous manner the following compounds were synthesized following the above procedure:

### Intermediate B

### 2-Phenethyl-tetrahydro-pyran-4-ol

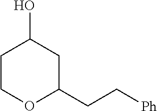 [SMILES: OC1CCOC(CCc2ccccc2)C1]

From but-3-en-1-ol and 3-phenylpropanal, the crude material was purified by column chromatography to afford 2-phenethyl-tetrahydro-pyran-4-ol as a brown oil (16.4 g, 69%). ¹H NMR (CDCl₃) δ: 7.15-7.33 (m, 5H), 4.06 (ddd, J=11.8, 4.8, 1.5 Hz, 1H), 3.67-3.83 (m, 1H), 3.39 (td, J=12.2, 2.1 Hz, 1H), 3.26 (dddd, J=12.8, 6.4, 4.2, 1.9 Hz, 1H), 2.58-2.87 (m, 2H), 1.81-2.02 (m, 3H), 1.62-1.81 (m, 1H), 1.10-1.35 (m, 2H)

### Intermediate C

### 2-sec-Butyl-tetrahydro-pyran-4-ol

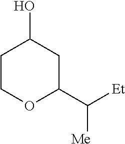 [SMILES: CCC(C)C1CC(O)CCO1]

From but-3-en-1-ol and 2-methyl-butyraldehyde, the crude material was purified by column chromatography to afford 2-sec-butyl-tetrahydro-pyran-4-ol as a brown solid (17.1 g, 94%). ¹H NMR (CDCl₃) δ: 3.96-4.07 (m, 1H), 3.70-3.84 (m, 1H), 3.37 (tt, J=12.1, 1.9 Hz, 1H), 3.05-3.17 (m, 1H), 1.81-1.99 (m, 1H), 1.43-1.64 (m, 3H), 1.09-1.33 (m, 2H), 0.84-0.95 (m, 5H)

### Intermediate D

### 2-Cyclopentyl-tetrahydro-pyran-4-ol

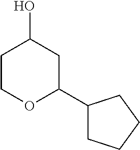 [SMILES: OC1CCOC(C2CCCC2)C1]

A solution of but-3-en-1-ol (5.95 g, 82.5 mmol) and cyclopentanecarbaldehyde (16.2 g, 165 mmol) in methylene chloride (200 mL) was cooled to 0° C. Trifluoroacetic acid (80 mL) was added slowly under nitrogen atmosphere. The reaction mixture was allowed to warm to room temperature and stirred for 18 h. The resulting mixture was concentrated in vacuo and azeotroped with toluene to ensure complete removal of any residual trifluoroacetic acid. The residue was dissolved in methanol (200 mL) and treated with sodium carbonate (70.0 g, 660 mmol). The reaction mixture was allowed to stir for 1.5 h. The resulting mixture was filtered and concentrated in vacuo. The residue was diluted with water and extracted with methylene chloride. The combined organic layer was dried over sodium sulfate, filtered, and concentrated in vacuo to give a crude oil (14.7 g, quant.) that was taken on without further purification. ¹H NMR (CDCl₃) δ: 4.02 (ddd, J=11.7, 4.9, 1.9 Hz, 1H), 3.69-3.83 (m, 1H), 3.38 (td, J=12.1, 2.3 Hz, 1H), 3.03 (ddd, J=11.0, 7.6, 1.7 Hz, 1H), 2.01 (ddt, J=12.2, 4.5, 2.0 Hz, 1H), 1.77-1.93 (m, 3H), 1.32-1.72 (m, 8H), 1.12-1.28 (m, 1H)

In an analogous manner the following compounds were synthesized following the above procedure:

### Intermediate E

### 2-tert-Butyl-tetrahydro-pyran-4-ol

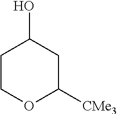

From but-3-en-1-ol and 2,2-dimethyl-propionaldehyde, the crude material was purified by column chromatography (SiO₂, 0% to 50% ethyl acetate in heptanes) to afford 2-tert-butyl-tetrahydro-pyran-4-ol as a slow forming solid (6.65 g, 76%). ¹H NMR (CDCl₃) δ: 4.04 (ddd, J=11.7, 4.9, 1.9 Hz, 1H), 3.77 (tt, J=10.9, 4.6 Hz, 1H), 3.35 (td, J=12.1, 1.9 Hz, 1H), 2.87 (dd, J=11.3, 1.9 Hz, 1H), 1.96 (ddt, J=12.1, 4.3, 2.0 Hz, 1H), 1.87 (ddq, J=12.5, 4.4, 2.0 Hz, 1H), 1.37-1.54 (m, 1H), 1.20 (q, J=11.2 Hz, 1H), 0.91 (s, 10H).

### Intermediate F

### 2-(1-Methyl-cyclohexyl)-tetrahydro-pyran-4-ol

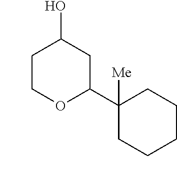 [SMILES: CC1(C2CC(O)CCO2)CCCCC1]

From but-3-en-1-ol and 1-methyl-cyclohexanecarbaldehyde, the crude material was purified by column chromatography to afford 2-(1-methyl-cyclohexyl)-tetrahydro-pyran-4-ol as a light yellow oil (4.34 g, 80%). ¹H NMR (CDCl₃) δ: 4.03 (ddd, J=11.7, 4.9, 1.9 Hz, 1H), 3.76 (tt, J=10.9, 4.6 Hz, 1H), 3.33 (td, J=12.1, 2.3 Hz, 1H), 2.95 (dd, J=11.3, 1.5 Hz, 1H), 1.78-2.00 (m, 3H), 1.23-1.51 (m, 11H), 0.88 (s, 3H)

### Intermediate G

### rac-6-Oxa-spiro[4.5]decan-9-ol

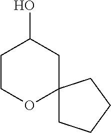 [SMILES: OC1CCOC2(CCCC2)C1]

From but-3-en-1-ol and cyclopentanone: 6-oxa-spiro[4.5]decan-9-ol was obtained as a crude brown oil (3.46 g, 19%). ¹H NMR (CDCl₃) δ: 3.71-3.97 (m, 2H), 3.54 (td, J=12.0, 2.5 Hz, 1H), 2.10-2.22 (m, 2H), 1.93-2.00 (m, 2H), 1.78-1.93 (m, 4H), 1.66-1.78 (m, 2H), 1.49-1.66 (m, 2H)

### Intermediate H

### 2-(2-Benzyloxy-1,1-dimethyl-ethyl)-tetrahydro-pyran-4-ol

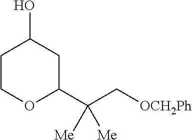

From but-3-en-1-ol and 3-benzyloxy-2,2-dimethyl-propionaldehyde: the crude material was purified by column chromatography (SiO₂, 0% to 40% ethyl acetate in heptanes) to afford 2-(2-benzyloxy-1,1-dimethyl-ethyl)-tetrahydro-pyran-4-ol as a viscous yellow oil (3.76 g, 68%). ¹H NMR (CDCl₃) δ: 7.28-7.45 (m, 5H), 4.50 (s, 2H), 4.00 (ddd, J=11.6, 4.8, 1.7 Hz, 1H), 3.68-3.86 (m, 1H), 3.34 (d, J=8.7 Hz, 2H), 3.35 (td, J=12.2, 2.1 Hz, 1H), 3.19 (d, J=8.7 Hz, 2H), 3.23 (dd, J=11.3, 1.5 Hz, 1H), 1.81-1.95 (m, 2H), 1.37-1.54 (m, 1H), 1.14-1.32 (m, 2H), 0.93 (s, 3H), 0.91 (s, 3H)

### Intermediate I

### 4-(4-Hydroxy-tetrahydro-pyran-2-yl)-piperidine-1-carboxylic acid tert-butyl ester

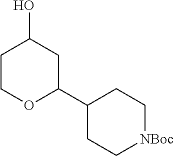 [SMILES: CC(C)(C)OC(=O)N1CCC(C2CC(O)CCO2)CC1]

A solution of but-3-en-1-ol (1.60 g, 22.2 mmol) and tert-butyl 4-formylpiperidine-1-carboxylate (9.46 g, 44.4 mmol) in methylene chloride (500 mL) was cooled to 0° C. Trifluoroacetic acid (200 mL) was added slowly under nitrogen atmosphere. The reaction mixture was allowed to warm to room temperature and stirred for 18 h. The resulting mixture was concentrated in vacuo. The crude material was dissolved in methylene chloride (200 mL) and treated with di-tert-butyl dicarbonate (10 g, 45.8 mmol) and triethylamine (46.4 ml, 333 mmol). The resulting mixture was stirred at room temperature overnight and subsequently concentrated in vacuo. The crude material was dissolved in methanol (500 mL) and treated with potassium carbonate (312 g, 22.2 mmol). The reaction mixture was stirred at room temperature for 3 h. The resulting mixture was diluted with water (250 mL) and extracted with methylene chloride. The organic layers were dried over magnesium sulfate and concentrated in vacuo. The crude residue was purified by column chromatography (SiO₂, 10% to 80% ethyl acetate in hexanes) to afford 4-(4-hydroxy-tetrahydro-pyran-2-yl)-piperidine-1-carboxylic acid tert-butyl ester as a colorless oil (5.00 g, 79%). ¹H NMR (CDCl₃) δ: 4.07-4.21 (m, 2H), 4.02 (ddd, J=11.7, 4.9, 1.5 Hz, 1H), 3.77 (tt, J=11.0, 4.7 Hz, 1H), 3.36 (td, J=12.2, 2.1 Hz, 1H), 3.03 (ddd, J=11.1, 6.6, 1.5 Hz, 1H), 2.66 (tdd, J=13.2, 4.5, 3.0 Hz, 2H), 1.77-2.03 (m, 3H), 1.48-1.68 (m, 3H), 1.45 (s, 9H), 1.09-1.31 (m, 3H)

In an analogous manner the following compound was synthesized following the above procedure:

### Intermediate J

### 3-(4-Hydroxy-tetrahydro-pyran-2-yl)-pyrrolidine-1-carboxylic acid tert-butyl ester

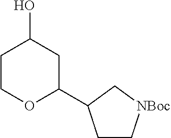 [SMILES: CC(C)(C)OC(=O)N1CCC(C2CC(O)CCO2)C1]

From 3-formyl-pyrrolidine-1-carboxylic acid tert-butyl ester: 3-(4-hydroxy-tetrahydro-pyran-2-yl)-pyrrolidine-1-carboxylic acid tert-butyl ester was obtained as an oil (2.38 g, 63%). ¹H NMR (CDCl₃) δ: 3.95-4.07 (m, 1H), 3.70-3.84 (m, 1H), 2.93-3.62 (m, 7H), 2.13-2.30 (m, 2H), 1.51-1.95 (m, 5H), 1.45 (s, 9H)

### Intermediate K

### rac-2-Isopropyl-tetrahydro-pyran-4-one

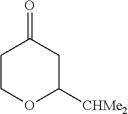

A solution of 2-isopropyl-tetrahydro-pyran-4-ol (14.9 g, 103 mmol), silica gel (60 mL) and PCC (28.9 g, 134 mmol) was stirred at room temperature overnight. The resulting dark mixture was filtered through a pad of silica and concentrated in vacuo to give 2-isopropyl-tetrahydro-pyran-4-one as a yellow oil (12.24 g, 83%). This material was taken on to the next step without further purification. ¹H-NMR (CDCl₃) δ: 4.31 (ddd, J=11.4, 7.5, 1.1 Hz, 1H), 3.63 (td, J=11.9, 3.0 Hz, 1H), 3.30 (ddd, J=11.1, 6.0, 2.8 Hz, 1H), 2.59 (ddd, J=13.6, 12.5, 7.6 Hz, 1H), 2.32-2.46 (m, 2H), 2.25-2.36 (m, 1H), 1.81 (dq, J=13.3, 6.7 Hz, 1H), 0.98 (d, J=6.8 Hz, 3H), 0.93 (d, J=6.8 Hz, 3H)

In an analogous manner the following compounds were synthesized following the above procedure:

### Intermediate L

### rac-2-Phenethyl-tetrahydro-pyran-4-one

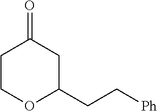 [SMILES: O=C1CCOC(CCc2ccccc2)C1]

From 2-phenethyl-tetrahydro-pyran-4-ol: 2-phenethyl-tetrahydro-pyran-4-ol was obtained as a pale green oil (9.15 g, 57%). ¹H NMR (CDCl₃) δ: 7.16-7.34 (m, 5H), 4.33 (ddd, J=11.5, 7.4, 1.1 Hz, 1H), 3.66 (td, J=11.9, 3.0 Hz, 1H), 3.52-3.60 (m, 1H), 2.52-2.91 (m, 3H), 2.23-2.51 (m, 3H), 1.99 (dtd, J=14.0, 8.6, 5.5 Hz, 1H), 1.80 (dddd, J=13.9, 9.5, 7.2, 4.2 Hz, 1H)

### Intermediate M

### 2-sec-Butyl-tetrahydro-pyran-4-one

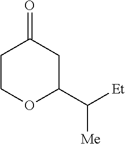 [SMILES: CCC(C)C1CC(=O)CCO1]

From 2-sec-butyl-tetrahydro-pyran-4-ol: 2-sec-butyl-tetrahydro-pyran-4-one was obtained as a yellow oil (5.31 g, 88%). ¹H NMR (CDCl₃) δ: 4.29 (dd, J=11.3, 7.6 Hz, 1H), 3.62 (tt, J=11.9, 2.6 Hz, 1H), 3.33-3.50 (m, 1H), 2.49-2.69 (m, 1H), 2.22-2.46 (m, 3H), 1.41-1.76 (m, 3H), 1.09-1.24 (m, 1H), 0.87-0.98 (m, 7H)

### Intermediate N

### rac-2-(1-Methyl-cyclohexyl)-tetrahydro-pyran-4-one

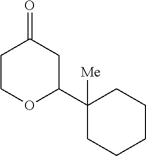 [SMILES: CC1(C2CC(=O)CCO2)CCCCC1]

From 2-(1-methyl-cyclohexyl)-tetrahydro-pyran-4-ol: 2-(1-methyl-cyclohexyl)-tetrahydro-pyran-4-one was obtained as a brown oil (2.50 g, 72%). ¹H NMR (CDCl₃) δ: 4.31 (ddd, J=11.4, 7.6, 0.9 Hz, 1H), 3.59 (ddd, J=12.5, 11.3, 2.6 Hz, 1H), 3.28 (dd, J=8.7, 5.3 Hz, 1H), 2.58 (ddd, J=14.6, 12.6, 7.6 Hz, 1H), 2.27-2.39 (m, 3H), 1.26-1.64 (m, 12H), 0.92 (s, 3H)

### Intermediate O

### rac-2-tert-Butyl-tetrahydro-pyran-4-one

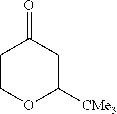

From 2-tert-butyl-tetrahydro-pyran-4-ol: 2-tert-butyl-tetrahydro-pyran-4-one was obtained as a light brown oil (5.21 g, 73%). ¹H NMR (CDCl₃) δ: 4.32 (ddd, J=11.4, 7.6, 0.9 Hz, 1H), 3.60 (ddd, J=12.5, 11.5, 2.8 Hz, 1H), 3.19 (dd, J=11.0, 3.0 Hz, 1H), 2.49-2.66 (m, 1H), 2.23-2.46 (m, 3H), 0.94 (s, 9H)

### Intermediate P

### 6-Oxa-spiro[4.5]decan-9-one

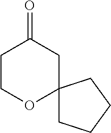 [SMILES: O=C1CCOC2(CCCC2)C1]

From 6-oxa-spiro[4.5]decan-9-ol the crude material was purified by column chromatography (SiO₂, 0% to 40% ethyl acetate in heptanes) to afford 6-oxa-spiro[4.5]decan-9-one as a colorless oil (1.75 g, 51%). ¹H NMR (DMSO-d₆) δ: 3.83 (t, J=6.2 Hz, 2H), 2.40 (s, 2H), 2.33 (t, J=6.4 Hz, 2H), 1.71-1.83 (m, 2H), 1.47-1.71 (m, 4H), 1.33-1.47 (m, 2H)

### Intermediate Q

### rac-2-(2-Benzyloxy-1,1-dimethyl-ethyl)-tetrahydro-pyran-4-one

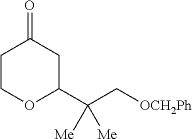

From 3-benzyloxy-2,2-dimethyl-propan-1-ol: 3-benzyloxy-2,2-dimethyl-propionaldehyde was obtained as a pale yellow oil (17.25 g, 73%). ¹H NMR (CDCl₃) δ: 9.58 (s, 1H), 7.28-7.40 (m, 5H), 4.52 (s, 2H), 3.46 (s, 2H), 1.10 (s, 6H).

A solution of 2,2-dimethyl-propane-1,3-diol (25.85 g, 248 mmol) in dioxane (400 mL) was cooled to 0° C. Potassium tert-butoxide (30 g, 267 mmol) was added portionwise to the cooled solution. The resulting mixture was stirred at room temperature for 1 h. (Bromomethyl)benzene (29.5 mL, 248 mmol) was added dropwise via addition funnel. The mixture was heated to 90° C. and stirred for 4 h. The resulting mixture was concentrated in vacuo. The resulting residue was partitioned between water (200 mL) and ethyl acetate (200 mL) and extracted with ethyl acetate (3×200 mL). The organics were combined, washed with brine (100 mL), dried on sodium sulfate, and concentrated in vacuo. The crude material was purified by column chromatography (SiO₂, 5% to 60% ethyl acetate in heptane) to afford 3-benzyloxy-2,2-dimethyl-propan-1-ol as a yellow oil (24.84 g, 51%). ¹H NMR (CDCl₃) δ: 7.28-7.42 (m, 5H), 4.52 (s, 2H), 3.47 (s, 2H), 3.34 (s, 2H), 0.94 (s, 6H). MS calcd. for C₁₂H₁₈O₂ [(M+H)⁺] 195.3, obsd. 195.

From 2-(2-benzyloxy-1,1-dimethyl-ethyl)-tetrahydro-pyran-4-ol the crude material was purified by column chromatography (SiO₂, 0% to 40% ethyl acetate in heptanes) to afford 2-(2-benzyloxy-1,1-dimethyl-ethyl)-tetrahydro-pyran-4-one as a colorless oil (3.54 g, 94%). ¹H NMR (CDCl₃) δ: 7.27-7.40 (m, 5H), 4.49 (d, J=2.6 Hz, 2H), 4.27 (dd, J=11.3, 7.6 Hz, 1H), 3.50-3.66 (m, 2H), 3.35 (d, J=8.7 Hz, 1H), 3.22 (d, J=8.7 Hz, 1H), 2.56 (ddd, J=14.5, 12.5, 7.7 Hz, 1H), 2.34-2.42 (m, 2H), 2.25-2.34 (m, 1H), 0.98 (s, 3H), 0.92 (s, 3H).

### Intermediate R

### rac-4-(4-Oxo-tetrahydro-pyran-2-ye-piperidine-1-carboxylic acid tert-butyl ester

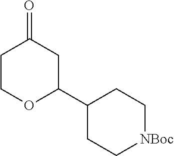 [SMILES: CC(C)(C)OC(=O)N1CCC(C2CC(=O)CCO2)CC1]

From 4-(4-hydroxy-tetrahydro-pyran-2-yl)-piperidine-1-carboxylic acid tert-butyl ester: 4-(4-oxo-tetrahydro-pyran-2-yl)-piperidine-1-carboxylic acid tert-butyl ester was obtained as a pale yellow solid (3.80 g, 70%). ¹H NMR (CDCl₃) δ: 4.30 (dd, J=11.5, 6.6 Hz, 1H), 4.16 (br. d, J=11.0 Hz, 2H), 3.56-3.68 (m, 1H), 3.35 (ddd, J=11.0, 6.3, 2.6 Hz, 1H), 2.49-2.77 (m, 3H), 2.22-2.48 (m, 3H), 1.88 (d, J=12.8 Hz, 1H), 1.55-1.69 (m, 2H), 1.46 (s, 9H), 1.11-1.33 (m, 2H).

### Intermediate S

### 3-(4-Oxo-tetrahydro-pyran-2-yl)-pyrrolidine-1-carboxylic acid tert-butyl ester

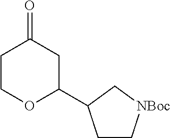 [SMILES: CC(C)(C)OC(=O)N1CCC(C2CC(=O)CCO2)C1]

From 3-(4-hydroxy-tetrahydro-pyran-2-yl)-pyrrolidine-1-carboxylic acid tert-butyl ester: 3-(4-oxo-tetrahydro-pyran-2-yl)-pyrrolidine-1-carboxylic acid tert-butyl ester was obtained as a viscous oil (1.90 g, 76%). ¹H NMR (CDCl₃) δ: 4.18-4.38 (m, 1H), 3.56-3.75 (m, 2H), 3.37-3.56 (m, 2H), 3.28 (d, J=7.2 Hz, 1H), 2.94-3.11 (m, 1H), 2.53-2.71 (m, 1H), 2.41-2.53 (m, 1H), 2.23-2.41 (m, 3H), 1.71-1.97 (m, 1H), 1.54-1.69 (m, 1H), 1.47 (s, 9H).

### Intermediate T

### rac-4-(9-Oxo-1,4,9,10-tetrahydro-2H-3-oxa-10-aza-phenanthren-2-yl)-piperidinium; chloride

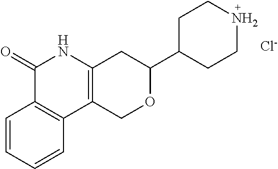

In a 10 mL pear-shaped flask, tert-butyl 4-(6-oxo-3,4,5,6-tetrahydro-1H-pyrano[4,3-c]isoquinolin-3-yl)piperidine-1-carboxylate (210 mg, 546 μmol, Eq: 1.00) was combined with trifluoroacetic acid (3.7 g, 2.5 mL, 32.4 mmol, Eq: 59.4) and dichloromethane (2.5 mL) to give a light brown solution. The mixture was stirred for 20 min and evaporated. The residue was re-evaporated twice from CH₃CN, twice from CH₃CN containing 5 drops 0.5 N HCl and once from CH₃CN to give 290 mg of 4-(9-oxo-1,4,9,10-tetrahydro-2H-3-oxa-10-aza-phenanthren-2-yl)-piperidinium chloride as a sticky, light brown solid. MS calcd. for C₁₇H₂₀N₂O₂ [(M+H)⁺] 285.4, obsd. 285.3.

### Intermediate U

### 5-Bromo-1-methyl-1H-pyrazole-4-carboxylic acid ethyl ester

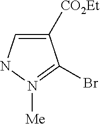

5-Amino-1-methyl-1H-pyrazole-4-carboxylic acid ethyl ester (5.0 g, 29.6 mmol) was added portionwise to a mixture of tert-butyl nitrite (4.57 g, 44.3 mmol) and copper (II) bromide (7.92 g, 35.5 mmol) in acetonitrile (20 mL). The mixture was heated to 60° C. for 2 h. The resulting mixture was poured into 6M HCl (200 mL) and extracted with dichloromethane (3×250 mL). The combined organics was dried on magnesium sulfate and concentrated in vacuo. The crude material was purified by column chromatography (SiO₂, 0% to 50% ethyl acetate in hexanes) to afford 5-bromo-1-methyl-1H-pyrazole-4-carboxylic acid ethyl ester as an off-white solid (4.7 g, 68%). ¹H NMR (CDCl₃) δ: 7.93 (s, 1H), 4.32 (q, J=7.2 Hz, 2H), 3.92 (s, 3H), 1.36 (t, J=7.0 Hz, 3H).

### Example 1

### rac-2-Isopropyl-1,2,4,10-tetrahydro-3-oxa-10-aza-phenanthren-9-one (I-2)

2-Isopropyl-1,2,4,10-tetrahydro-3-oxa-10-aza-phenanthren-9-one was synthesized following the procedure in Example 7. From 2-bromo-benzoic acid methyl ester and 2-isopropyl-tetrahydro-pyran-4-one: 2-isopropyl-1,2,4,10-tetrahydro-3-oxa-10-aza-phenanthren-9-one was obtained as an off-white solid (25 mg, 4%). ¹H NMR (MeOH-d₄) δ: 8.32 (d, J=8.3 Hz, 1H), 7.69-7.78 (m, 1H), 7.41-7.57 (m, 2H), 5.02 (d, J=14.4 Hz, 1H), 4.75 (ddd, J=14.4, 5.3, 2.5 Hz, 1H), 3.39-3.50 (m, 1H), 2.55-2.63 (m, 2H), 1.84 (dqd, J=13.6, 6.7, 3.2 Hz, 1H), 1.09 (dd, J=6.8, 3.0 Hz, 3H), 1.03 (dd, J=6.8, 3.0 Hz, 4H). MS calcd. for C₁₅H₁₇NO₂ [(M+H)^{+]} 244.3, obsd. 244.

10-Fluoro-3-isopropyl-1 3 4 5-tetrahydropyrano[4,3-c]isoquinolin-6-one (1-69) and 10-chloro-3-isopropyl-1 3 4 5-tetrahydropyrano[4,3-c]isoquinolin-6-one (1-70) were prepared analogously except 2-bromo-benzoic acid methyl ester was replaced with methyl 2-bromo-3-fluoro-benzoate and methyl 2-bromo-3-chloro-benzoate respectively. The parent ions observed for I-69 and I-70 were m/e=262.15 and m/e=278.0 respectively.

### Example 2

### 1,2,4,10-Tetrahydro-3-oxa-10-aza-phenanthren-9-one (I-3)

1,2,4,10-Tetrahydro-3-oxa-10-aza-phenanthren-9-one was synthesized following the procedure in Example 7. From tetrahydro-pyran-4-one and 2-bromo-benzoic acid methyl ester: 1,2,4,10-tetrahydro-3-oxa-10-aza-phenanthren-9-one was obtained as an off-white powder (34 mg, 2%). ¹H NMR (DMSO-d₆) δ: 11.25 (br. s., 1H), 8.19 (d, J=7.9 Hz, 1H), 7.68 (t, J=7.6 Hz, 1H), 7.45 (t, J=7.9 Hz, 1H), 7.40 (d, J=7.9 Hz, 1H), 4.70 (s, 2H), 3.88 (t, J=5.5 Hz, 2H), 2.57 (t, J=5.7 Hz, 2H). MS calcd. for C₁₂H₁₁NO₂ [(M+H)⁺] 202.2, obsd. 202.1.

### Example 3

### rac-2-Isopropyl-1,2,4,10-tetrahydro-3-oxa-5,10-diaza-phenanthren-9-one (I-4)

2-Isopropyl-1,2,4,10-tetrahydro-3-oxa-5,10-diaza-phenanthren-9-one was synthesized following the procedure in Example 7. From 2-isopropyl-tetrahydro-pyran-4-one and 2-bromo-nicotinic acid methyl ester: 2-isopropyl-1,2,4,10-tetrahydro-3-oxa-5,10-diaza-phenanthren-9-one was obtained as an off-white solid (126 mg, 24%). ¹H NMR (DMSO-d₆) δ: 11.48 (br. s., 1H), 8.86 (dd, J=4.5, 1.9 Hz, 1H), 8.46 (dd, J=7.9, 1.9 Hz, 1H), 7.45 (dd, J=7.9, 4.5 Hz, 1H), 5.03 (d, J=14.7 Hz, 1H), 4.60 (dt, J=14.9, 2.2 Hz, 1H), 3.41 (q, J=6.7 Hz, 1H), 1.78 (dq, J=13.4, 6.6 Hz, 1H), 0.93 (d, J=6.8 Hz, 3H), 0.97 (d, J=6.4 Hz, 3H). MS calcd. for C₁₄H₁₆N₂O₂ [(M+H)⁺] 244.3, obsd. 245.

### Example 4

### rac-7-Isopropyl-5,6,7,9-tetrahydro-8-oxa-3-thia-5-aza-cyclopenta[a]naphthalen-4-one (I-5)

7-Isopropyl-5,6,7,9-tetrahydro-8-oxa-3-thia-5-aza-cyclopenta[a]naphthalen-4-one was synthesized following the procedure in Example 7. From 2-isopropyl-tetrahydro-pyran-4-one and 3-bromo-thiophene-2-carboxylic acid methyl ester: 7-isopropyl-5,6,7,9-tetrahydro-8-oxa-3-thia-5-aza-cyclopenta[a]naphthalen-4-one was obtained as an off-white solid (247 mg, 24%). ¹H NMR (DMSO-d₆) δ: 11.42 (s, 1H), 8.03 (d, J=5.3 Hz, 1H), 7.25 (d, J=4.9 Hz, 1H), 4.83 (d, J=14.4 Hz, 1H), 4.59 (d, J=14.7 Hz, 1H), 3.37 (ddd, J=9.1, 6.4, 4.9 Hz, 1H), 1.76 (dq, J=13.4, 6.7 Hz, 1H), 0.96 (d, J=6.8 Hz, 3H), 0.92 (d, J=6.8 Hz, 3H). MS calcd. for C₁₃H₁₅NO₂S [(M+H)⁺] 250.3, obsd. 250.

### Example 5

### rac-7-Chloro-2-isopropyl-1,2,4,10-tetrahydro-3-oxa-10-aza-phenanthren-9-one (I-6)

7-Chloro-2-isopropyl-1,2,4,10-tetrahydro-3-oxa-10-aza-phenanthren-9-one was synthesized following the procedure in Example 7. From 2-isopropyl-tetrahydro-pyran-4-one and 2-bromo-5-chloro-benzoic acid methyl ester: 7-chloro-2-isopropyl-1,2,4,10-tetrahydro-3-oxa-10-aza-phenanthren-9-one was obtained as a white solid (151 mg, 53%). ¹H NMR (DMSO-d₆) δ: 11.41 (s, 1H), 8.11 (d, J=2.6 Hz, 1H), 7.73 (dd, J=8.7, 2.6 Hz, 1H), 7.45 (d, J=8.7 Hz, 1H), 4.89 (d, J=14.4 Hz, 1H), 4.57 (d, J=14.4 Hz, 1H), 3.37 (q, J=6.7 Hz, 1H), 1.68-1.83 (m, 1H), 0.97 (d, J=6.8 Hz, 4H), 0.92 (d, J=6.8 Hz, 3H). MS calcd. for C₁₅H₁₆ClNO₂ [(M+H)⁺] 278.7, obsd. 278.

### Example 6

### rac-2-Cyclopentyl-1,2,4,10-tetrahydro-3-oxa-10-aza-phenanthren-9-one (I-7)

2-Cyclopentyl-1,2,4,10-tetrahydro-3-oxa-10-aza-phenanthren-9-one was synthesized following the procedure in Example 7. From 2-isopropyl-tetrahydro-pyran-4-one and 2-bromo-benzoic acid methyl ester: 2-cyclopentyl-1,2,4,10-tetrahydro-3-oxa-10-aza-phenanthren-9-one was obtained as a white solid (180 mg, 38%). ¹H NMR (DMSO-d₆) δ: 11.20 (br. s., 1H), 8.18 (d, J=7.9 Hz, 1H), 7.59-7.78 (m, 1H), 7.44 (t, J=7.7 Hz, 1H), 7.39 (d, J=7.9 Hz, 1H), 4.88 (d, J=14.4 Hz, 1H), 4.60 (d, J=14.4 Hz, 1H), 3.37-3.52 (m, 1H), 2.32-2.61 (m, 2H), 1.99 (sxt, J=7.9 Hz, 1H), 1.73-1.82 (m, 1H), 1.38-1.71 (m, 6H), 1.15-1.35 (m, 1H). MS calcd. for C₁₇H₁₉NO₂ [(M+H)⁺] 270.4, obsd. 270.

### Example 7

### rac-2-tert-Butyl-1,2,4,10-tetrahydro-3-oxa-10-aza-phenanthren-9-one (I-8)

A mixture of 2-tert-butyldihydro-2H-pyran-4(3H)-one (0.3 g, 1.92 mmol) xantphos (22.2 mg, 38.4 μmol) and methyl 2-bromobenzoate (330 mg, 1.54 mmol), cesium carbonate (813 mg, 2.5 mmol), and Pd₂(dba)₃ (17.6 mg, 19.2 mmol), in toluene (2.0 mL) was placed under argon atmosphere and stirred at 90° C. for 15 h. The reaction mixture was pre-absorbed onto silica and purified by flash chromatography (SiO₂, 0% to 50% EtOAc in hexanes) to afford a crude oil (137 mg). The crude oil was dissolved in a solution of ammonia in methanol (7M, 2 mL). The reaction mixture was heated at 140° C. in a microwave reactor for 1 h. The precipitate was filtered and dried in vacuo to afford 2-tert-butyl-1,2,4,10-tetrahydro-3-oxa-10-aza-phenanthren-9-one as a white solid (34 mg, 7%). ¹H NMR (DMSO-d₆) δ: 11.19 (br. s, 1H), 8.18 (dd, J=7.9, 1.1 Hz, 1H), 7.61-7.77 (m, 1H), 7.40 (d, J=7.9 Hz, 1H), 7.45 (d, J=7.6 Hz, 1H), 4.94 (d, J=14.0 Hz, 1H), 4.59 (dt, J=14.2, 2.2 Hz, 1H), 3.30-3.37 (m, 1H), 2.36-2.55 (m, 2H), 0.94 (s, 9H). MS calcd. for C₁₆H₁₉NO₂ [(M+H)⁺] 258.4, obsd. 258.1.

In an analogous manner the following compounds (Example 8—were synthesized following the above procedure:

### Example 8

### 1,5′-Dihydrospiro[cyclopentane-1,3′-pyrano[4,3-c]isoquinolin]-6′(4′H)-one (I-9)

1′,5′-Dihydrospiro[cyclopentane-1,3′-pyrano[4,3-c]isoquinolin]-6′(4′H)-one was synthesized following the procedure in Example 7. From 6-oxa-spiro[4.5]decan-9-one and methyl 2-bromobenzoate: 1′,5′-Dihydrospiro[cyclopentane-1,3′-pyrano[4,3-c]isoquinolin]-6′(4′H)-one was obtained as off-white needles (100 mg, 20%) ¹H NMR (DMSO-d₆) δ: 11.21 (br. s., 1H), 8.18 (d, J=7.9 Hz, 1H), 7.59-7.79 (m, 1H), 7.46 (d, J=7.6 Hz, 1H), 7.41 (d, J=7.9 Hz, 1H), 4.69 (s, 2H), 2.57 (s, 2H), 1.78-1.91 (m, 2H), 1.57-1.76 (m, 4H), 1.41-1.55 (m, 2H). MS calcd. for C₁₆H₁₇NO₂ [(M+H)⁺] 256.3, obsd. 256.

### Example 9

### rac-7-Isopropyl-5,6,7,9-tetrahydro-1,8-dioxa-5-aza-cyclopenta[a]naphthalen-4-one (I-10)

7-Isopropyl-5,6,7,9-tetrahydro-1,8-dioxa-5-aza-cyclopenta[a]naphthalen-4-one was synthesized following the procedure in Example 7. From 2-isopropyl-tetrahydro-pyran-4-one and 2-bromo-furan-3-carboxylic acid methyl ester, the crude material was purified by column chromatography (SiO₂, 0% to 100% ethyl acetate in hexanes) 7-isopropyl-5,6,7,9-tetrahydro-1,8-dioxa-5-aza-cyclopenta[a]naphthalen-4-one was obtained as a pale yellow solid (15 mg, 3%). ¹H NMR (DMSO-d₆) δ: 11.38 (s, 1H), 7.83 (d, J=2.0 Hz, 1H), 6.91 (d, J=2.3 Hz, 1H), 4.83 (d, J=14.1 Hz, 1H), 4.60 (dt, J=14.3, 2.2 Hz, 1H), 3.40 (ddd, J=10.2, 6.4, 4.0 Hz, 2H), 2.42-2.48 (m, 1H), 1.78 (dq, J=13.5, 6.7 Hz, 1H), 0.97 (d, J=6.6 Hz, 3H), 0.93 (d, J=7.1 Hz, 3H). MS calcd. for C₁₃H₁₅NO₃ [(M+H)⁺] 233.3, obsd. 234.

### Example 10

### (S)-2-Cyclopentyl-1,2,4,10-tetrahydro-3-oxa-10-aza-phenanthren-9-one (I-11)

The racemic 2-cyclopentyl-1,2,4,10-tetrahydro-3-oxa-10-aza-phenanthren-9-one was chromatographed using SFC separation OD column to give two optically pure isomers. The first peak was pooled and evaporated to yield (S)-2-Cyclopentyl-1,2,4,10-tetrahydro-3-oxa-10-aza-phenanthren-9-one. ¹H NMR (DMSO-d₆) δ: 11.20 (s, 1H), 8.18 (d, J=7.2 Hz, 1H), 7.68 (td, J=7.7, 1.2 Hz, 1H), 7.44 (t, J=7.6 Hz, 1H), 7.40 (d, J=8.3 Hz, 1H), 4.89 (d, J=14.4 Hz, 1H), 4.60 (d, J=14.4 Hz, 1H), 3.39-3.51 (m, 1H), 2.00 (q, J=8.3 Hz, 1H), 1.72-1.86 (m, 1H), 1.36-1.72 (m, 6H), 1.17-1.36 (m, 1H).

### Example 11

### (R)-2-Cyclopentyl-1,2,4,10-tetrahydro-3-oxa-10-aza-phenanthren-9-one (I-12)

From the SFC separation above, the second peak was pooled and evaporated to yield (R)-2-cyclopentyl-1,2,4,10-tetrahydro-3-oxa-10-aza-phenanthren-9-one. ¹H NMR (DMSO-d₆) δ: 11.20 (s, 1H), 8.18 (d, J=7.2 Hz, 1H), 7.68 (td, J=7.7, 1.0 Hz, 1H), 7.44 (t, J=7.6 Hz, 1H), 7.40 (d, J=7.9 Hz, 1H), 4.89 (d, J=14.0 Hz, 1H), 4.60 (d, J=14.4 Hz, 1H), 3.37-3.53 (m, 1H), 2.00 (q, J=7.6 Hz, 1H), 1.71-1.90 (m, 1H), 1.36-1.71 (m, 6H), 1.19-1.36 (m, 1H).

### Example 12

### rac-2-Phenethyl-1,2,4,10-tetrahydro-3-oxa-10-aza-phenanthren-9-one (I-13)

2-Phenethyl-1,2,4,10-tetrahydro-3-oxa-10-aza-phenanthren-9-one was synthesized following the procedure in Example 7. From 2-phenethyl-tetrahydro-pyran-4-ol and methyl 2-bromobenzoate: 2-phenethyl-1,2,4,10-tetrahydro-3-oxa-10-aza-phenanthren-9-one was obtained as a white solid (99 mg, 16%). ¹H NMR (DMSO-d₆) δ: 11.24 (br. s., 1H), 8.18 (dd, J=8.1, 1.3 Hz, 1H), 7.69 (td, J=7.6, 1.5 Hz, 1H), 7.36-7.54 (m, 2H), 7.04-7.36 (m, 5H), 4.92 (d, J=14.4 Hz, 1H), 4.62 (dt, J=14.4, 2.1 Hz, 1H), 3.52-3.75 (m, 1H), 2.60-2.92 (m, 2H), 1.80-1.98 (m, 2H). MS calcd. for C₂₀H₁₉NO₂ [(M+H)⁺] 306.4, obsd. 306.

### Example 13

### rac-7-Isopropyl-5,7,8,9-tetrahydro-6-oxa-3,9-diaza-phenanthren-10-one (I-14)

7-Isopropyl-5,7,8,9-tetrahydro-6-oxa-3,9-diaza-phenanthren-10-one was synthesized following the procedure in Example 7. From 2-isopropyl-tetrahydro-pyran-4-one and 3-bromo-isonicotinic acid methyl ester: 7-isopropyl-5,7,8,9-tetrahydro-6-oxa-3,9-diaza-phenanthren-10-one was obtained as a pale yellow solid (28 mg, 5%). ¹H NMR (DMSO-d₆) δ: 11.40 (br. s., 1H), 8.88 (s, 1H), 8.61 (d, J=5.3 Hz, 1H), 7.98 (d, J=5.3 Hz, 1H), 5.06 (d, J=14.7 Hz, 1H), 4.67 (dt, J=14.6, 2.3 Hz, 1H), 3.39 (q, J=6.5 Hz, 1H), 1.77 (dq, J=13.6, 6.8 Hz, 1H), 0.98 (d, J=6.4 Hz, 3H), 0.93 (d, J=6.8 Hz, 3H). MS calcd. for C₁₄H₁₆N₂O₂ [(M+H)⁺] 245.3, obsd. 245.

### Example 14

### rac-7-Isopropyl-5,7,8,9-tetrahydro-6-oxa-2,9-diaza-phenanthren-10-one (I-15)

7-Isopropyl-5,7,8,9-tetrahydro-6-oxa-2,9-diaza-phenanthren-10-one was synthesized following the procedure in Example 7. From 2-isopropyl-tetrahydro-pyran-4-one and 4-bromo-nicotinic acid methyl ester: 7-isopropyl-5,7,8,9-tetrahydro-6-oxa-2,9-diaza-phenanthren-10-one was obtained as a grey solid (3 mg, 1%). ¹H NMR (DMSO-d₆) δ: 11.40 (br. s., 1H), 8.88 (s, 1H), 8.61 (d, J=5.3 Hz, 1H), 7.98 (d, J=5.3 Hz, 1H), 5.06 (d, J=14.7 Hz, 1H), 4.67 (dt, J=14.6, 2.3 Hz, 1H), 3.39 (q, J=6.5 Hz, 1H), 1.77 (dq, J=13.6, 6.8 Hz, 1H), 0.98 (d, J=6.4 Hz, 3H), 0.93 (d, J=6.8 Hz, 3H). MS calcd. for C₁₄H₁₆N₂O₂ [(M+H)⁺] 245.3, obsd. 245.

### Example 15

### rac-2-Isopropyl-6-nitro-1,2,4,10-tetrahydro-3-oxa-10-aza-phenanthren-9-one (1-16)

2-Isopropyl-6-nitro-1,2,4,10-tetrahydro-3-oxa-10-aza-phenanthren-9-one was synthesized following the procedure in Example 7. From 2-isopropyl-tetrahydro-pyran-4-one and 2-bromo-4-nitro-benzoic acid methyl ester: 2-isopropyl-6-nitro-1,2,4,10-tetrahydro-3-oxa-10-aza-phenanthren-9-one was obtained as a yellow solid (28 mg, 2%). ¹H NMR (DMSO-d₆) δ: 11.73 (br. s., 1H), 8.89 (d, J=2.3 Hz, 1H), 8.43 (dd, J=8.9, 2.5 Hz, 1H), 7.63 (d, J=9.1 Hz, 1H), 4.96 (d, J=14.4 Hz, 1H), 4.62 (d, J=14.4 Hz, 1H), 3.36-3.47 (m, 1H), 1.78 (dq, J=13.2, 6.7 Hz, 1H), 0.98 (d, J=6.8 Hz, 3H), 0.93 (d, J=6.8 Hz, 3H). MS calcd. for C₁₅H₁₆N₂O₄ [(M+H)⁺] 289.3, obsd. 289.

### Example 16

### rac-2-Isopropyl-5-methyl-1,2,4,10-tetrahydro-3-oxa-10-aza-phenanthren-9-one (I-17)

2-Isopropyl-5-methyl-1,2,4,10-tetrahydro-3-oxa-10-aza-phenanthren-9-one was synthesized following the procedure in Example 7. From 2-isopropyl-tetrahydro-pyran-4-one and 2-bromo-3-methyl-benzoic acid methyl ester: 2-isopropyl-5-methyl-1,2,4,10-tetrahydro-3-oxa-10-aza-phenanthren-9-one was obtained as a colorless crystalline solid (72 mg, 13%). ¹H NMR (DMSO-d₆) δ: 11.17 (br. s., 1H), 8.11 (dd, J=7.7, 1.3 Hz, 1H), 7.45 (d, J=6.8 Hz, 1H), 7.29 (t, J=7.7 Hz, 1H), 5.12 (d, J=14.0 Hz, 1H), 4.89 (dt, J=14.0, 2.3 Hz, 1H), 3.28-3.34 (m, 1H), 2.58 (s, 3H), 1.72 (sxt, J=6.7 Hz, 1H), 0.97 (d, J=6.8 Hz, 3H), 0.92 (d, J=6.8 Hz, 3H). MS calcd. for C₁₆H₁₉NO₂ [(M+H)⁺] 258.3, obsd. 258.

### Example 17

### 2-sec-Butyl-1,2,4,10-tetrahydro-3-oxa-10-aza-phenanthren-9-one (I-18)

2-sec-Butyl-1,2,4,10-tetrahydro-3-oxa-10-aza-phenanthren-9-one was synthesized following the procedure in Example 7. From 2-sec-butyl-tetrahydro-pyran-4-one and methyl 2-bromobenzoate: 2-sec-butyl-1,2,4,10-tetrahydro-3-oxa-10-aza-phenanthren-9-one was obtained as colorless needles (50 mg, 5%). ¹H NMR (DMSO-d₆) δ: 11.21 (br. s., 1H), 8.18 (dd, J=7.9, 1.1 Hz, 1H), 7.68 (td, J=7.6, 1.1 Hz, 1H), 7.44 (t, J=7.6 Hz, 1H), 7.40 (d, J=8.3 Hz, 1H), 4.90 (d, J=14.4 Hz, 1H), 4.59 (d, J=14.4 Hz, 1H), 3.41-3.56 (m, 1H), 1.41-1.69 (m, 2H), 1.19 (tt, J=14.5, 7.6 Hz, 1H), 0.83-0.98 (m, 6H). MS calcd. for C₁₆H₁₉NO₂ [(M+H)⁺] 258.3, obsd. 258.

### Example 18

### rac-2-Isopropyl-9-oxo-1,4,9,10-tetrahydro-2H-3-oxa-10-aza-phenanthrene-6-carboxylic acid methyl ester (I-19)

2-Isopropyl-9-oxo-1,4,9,10-tetrahydro-2H-3-oxa-10-aza-phenanthrene-6-carboxylic acid methyl ester was synthesized following the procedure in Example 7. From 2-isopropyl-tetrahydro-pyran-4-one and 2-bromo-terephthalic acid dimethyl ester: 2-isopropyl-9-oxo-1,4,9,10-tetrahydro-2H-3-oxa-10-aza-phenanthrene-6-carboxylic acid methyl ester was obtained as colorless needles (25 mg, 4%). 1H NMR (DMSO-d6) δ: 11.45 (br. s., 1H), 8.30 (d, J=8.3 Hz, 1H), 7.92 (s, 1H), 7.94 (d, J=8.7 Hz, 1H), 4.96 (d, J=14.4 Hz, 1H), 4.66 (d, J=14.0 Hz, 1H), 3.90 (s, 3H), 3.34-3.45 (m, 1H), 1.78 (dq, J=13.5, 6.7 Hz, 1H), 0.95 (d, J=7.9 Hz, 3H), 0.98 (d, J=6.8 Hz, 3H), 0.93 (d, J=6.8 Hz, 3H). MS calcd. for C_{I7}H₁₉NO₄ [(M+H)⁺] 302.4, obsd. 302.

### Example 19

### rac-2-(1-Methyl-cyclohexyl)-1,2,4,10-tetrahydro-3-oxa-10-aza-phenanthren-9-one (I-20)

2-(1-Methyl-cyclohexyl)-1,2,4,10-tetrahydro-3-oxa-10-aza-phenanthren-9-one was synthesized following the procedure in Example 7. From 2-(1-methyl-cyclohexyl)-tetrahydro-pyran-4-one and 2-bromo-benzoic acid methyl ester: 2-(1-methyl-cyclohexyl)-1,2,4,10-tetrahydro-3-oxa-10-aza-phenanthren-9-one was obtained as a white powder (21 mg, 3%). ¹H NMR (DMSO-d₆) δ: 11.20 (s, 1H), 8.18 (dd, J=7.9, 1.1 Hz, 1H), 7.68 (td, J=7.8, 1.0 Hz, 1H), 7.44 (t, J=7.6 Hz, 1H), 7.39 (d, J=7.9 Hz, 1H), 4.94 (d, J=14.4 Hz, 1H), 4.58 (d, J=14.4 Hz, 1H), 3.44 (dd, J=11.0, 3.0 Hz, 1H), 2.57 (d, J=18.1 Hz, 1H), 2.37 (d, J=16.2 Hz, 1H), 1.30-1.60 (m, 8H), 1.05-1.30 (m, 2H), 0.91 (s, 3H). MS calcd. for C₁₉H₂₃NO₂ [(M+H)⁺] 298.4, obsd. 298.

### Example 20

### rac-4-(9-Oxo-1,4,9,10-tetrahydro-2H-3-oxa-10-aza-phenanthren-2-ye-piperidine-1-carboxylic acid tert-butyl ester (I-21)

4-(9-Oxo-1,4,9,10-tetrahydro-2H-3-oxa-10-aza-phenanthren-2-yl)-piperidine-1-carboxylic acid tert-butyl ester was synthesized following the procedure in Example 7. From 4-(4-oxo-tetrahydro-pyran-2-yl)-piperidine-1-carboxylic acid tert-butyl ester and 2-bromo-benzoic acid methyl ester: 4-(9-oxo-1,4,9,10-tetrahydro-2H-3-oxa-10-aza-phenanthren-2-ye-piperidine-1-carboxylic acid tert-butyl ester was obtained as a light yellow solid (357 mg, 11%). ¹H NMR (DMSO-d₆) δ: 11.24 (br. s., 1H), 8.18 (dd, J=7.9, 1.1 Hz, 1H), 7.68 (td, J=8.0, 1.0 Hz, 1H), 7.45 (t, J=7.7 Hz, 1H), 7.40 (d, J=8.3 Hz, 1H), 4.90 (d, J=14.4 Hz, 1H), 4.59 (d, J=14.7 Hz, 1H), 3.98 (d, J=12.8 Hz, 2H), 3.39-3.52 (m, 1H), 2.69 (br. s., 2H), 1.90 (d, J=15.1 Hz, 1H), 1.64 (d, J=7.2 Hz, 1H), 1.57 (d, J=13.6 Hz, 1H), 1.39 (s, 9H), 1.06-1.26 (m, 2H). MS calcd. for C₁₈H₂₀N₂O₄ [(M-tBu+H)⁺] 329.4, obsd. 329.1.

### Example 21

### rac-2-Piperidin-4-yl-1,2,4,10-tetrahydro-3-oxa-10-aza-phenanthren-9-one, trifluoroacetate (I-22)

A solution of 4-(9-oxo-1,4,9,10-tetrahydro-2H-3-oxa-10-aza-phenanthren-2-yl)-piperidine-1-carboxylic acid tert-butyl, ester (50 mg, 0.130 mmol) in trifluoroacetic acid (1 mL) was stirred at 140° C. for 1 hr. The resulting mixture was concentrated in vacuo to afford 2-piperidin-4-yl-1,2,4,10-tetrahydro-3-oxa-10-aza-phenanthren-9-one trifluoroacetate as an off-white solid (40 mg, 77%) that was taken to subsequent reactions without further purification. ¹H NMR (DMSO-d₆) δ: 11.29 (s, 1H), 8.57 (d, J=10.6 Hz, 1H), 8.25 (d, J=9.4 Hz, 1H), 8.19 (d, J=7.6 Hz, 1H), 7.69 (td, J=7.6, 1.1 Hz, 1H), 7.46 (t, J=7.7 Hz, 1H), 7.41 (d, J=7.9 Hz, 1H), 4.92 (d, J=14.4 Hz, 1H), 4.62 (d, J=14.4 Hz, 1H), 3.47-3.59 (m, 1H), 3.32 (d, J=11.7 Hz, 2H), 2.87 (q, J=11.2 Hz, 2H), 2.03 (d, J=14.0 Hz, 1H), 1.62-1.89 (m, 2H), 1.29-1.62 (m, 2H). MS calcd. for C₁₇H₂₀N₂O₂ [(M+H)⁺] 285.4, obsd. 285.

### Example 22

### rac-2-Isopropyl-9-oxo-1,4,9,10-tetrahydro-2H-3-oxa-10-aza-phenanthrene-6-carboxylic acid amide (I-23)

2-Isopropyl-9-oxo-1,4,9,10-tetrahydro-2H-3-oxa-10-aza-phenanthrene-6-carboxylic acid amide was synthesized following the procedure in Example 7. From 2-isopropyl-tetrahydro-pyran-4-one and 2-bromo-terephthalic acid dimethyl ester: 2-isopropyl-9-oxo-1,4,9,10-tetrahydro-2H-3-oxa-10-aza-phenanthrene-6-carboxylic acid amide was obtained as a white solid (12 mg, 1%). 1H NMR (DMSO-d6) δ: 11.34 (s, 1H), 8.22 (d, J=8.3 Hz, 2H), 8.18 (s, 1H), 7.88 (d, J=1.5 Hz, 1H), 7.84 (s, 1H), 7.59 (s, 1H), 4.96 (d, J=14.4 Hz, 1H), 4.64 (d, J=14.4 Hz, m), 3.39 (q, J=6.8 Hz, 1H), 1.78 (dq, J=13.3, 6.5 Hz, 1H), 0.98 (d, J=6.8 Hz, 3H), 0.93 (d, J=6.8 Hz, 3H).

### Example 23

### rac-6-Amino-2-isopropyl-1,2,4,10-tetrahydro-3-oxa-10-aza-phenanthren-9-one (I-24)

A mixture of 2-isopropyl-6-nitro-1,2,4,10-tetrahydro-3-oxa-10-aza-phenanthren-9-one (24 mg, 83.2 μmol) in ethanol (0.62 mL) and water (0.21 mL) was heated to 60° C. Ammonium chloride (4.45 mg, 0.084 mmol) and iron (18.6 mg, 0.33 mmol) were added to the heated suspension. The reaction mixture was heated and stirred for an additional 16 h. The resulting suspension was filtered through pad of celite and the pad was washed with copious amount of hot solution of methanol/ethyl acetate (1:1). The filtrate was concentrated in vacuo, triturated with water, and the resulting brown solid filtered. The crude solid was dissolved in a solution of methanol/methylene chloride and filtered through a Gelman Acrodisc syringe filter (LC13, PVDF, 0.45 um). The filtrate was concentrated in vacuo to afford 6-amino-2-isopropyl-1,2,4,10-tetrahydro-3-oxa-10-aza-phenanthren-9-one as a light brown solid (4 mg, 19%). ¹H NMR (DMSO-d₆) δ: 10.93 (s, 1H), 7.47 (d, J=2.3 Hz, 1H), 7.23 (d, J=8.7 Hz, 1H), 7.10 (dd, J=8.7, 2.6 Hz, 1H), 4.89 (d, J=14.4 Hz, 1H), 4.61 (d, J=14.4 Hz, 1H), 2.46 (d, J=5.7 Hz, 2H), 1.82 (dq, J=13.4, 6.6 Hz, 1H), 1.04 (d, J=6.8 Hz, 3H), 0.99 (d, J=6.8 Hz, 3H). MS calcd. for C₁₅H₁₈N₂O₂ [(M+H)⁺] 259.3, obsd. 259.

### Example 24

### rac-2-(1-Acetyl-piperidin-4-yl)-1,2,4,10-tetrahydro-3-oxa-10-aza-phenanthren-9-one (1-25)

Acetyl chloride (14.2 mg, 0.181 mmol) was added dropwise to a mixture of 2-piperidin-4-yl-1,2,4,10-tetrahydro-3-oxa-10-aza-phenanthren-9-one trifluoroacetate (60 mg, 0.151 mmol) in NMR (1 mL). The mixture was stirred at room temperature for 18 h. The resulting mixture was poured onto 1M NaOH (5 mL) and extracted with ethyl acetate (2×50 mL). The organics were combined and concentrated in vacuo to afford 2-(1-acetyl-piperidin-4-yl)-1,2,4,10-tetrahydro-3-oxa-10-aza-phenanthren-9-one as a white solid (50 mg, 91%). ¹H NMR (DMSO-d₆) δ: 11.24 (s, 1H), 8.18 (d, J=6.8 Hz, 1H), 7.68 (t, J=6.8 Hz, 1H), 7.45 (t, J=7.6 Hz, 1H), 7.40 (d, J=7.9 Hz, 1H), 4.90 (d, J=14.4 Hz, 1H), 4.60 (d, J=14.4 Hz, 1H), 4.42 (br. d, J=12.5 Hz, 1H), 3.84 (br. d, J=12.8 Hz, 1H), 3.38-3.56 (m, 1H), 2.88-3.12 (m, 1H), 1.83-2.02 (m, 1H), 1.98 (s, 3H), 1.73 (br. s., 1H), 1.61 (br. t, J=11.9 Hz, 1H), 1.18 (br. s., 4H). MS calcd. for C₁₉H₂₂N₂O₃ [(M+H)⁺] 327.4, obsd. 327.

### Example 25

### rac-2-(1-Methanesulfonyl-piperidin-4-yl)-1,2,4,10-tetrahydro-3-oxa-10-aza-phenanthren-9-one (I-26)

Methanesulfonyl chloride (20.7 mg, 14.1 μL, 181 μmol, Eq: 1.20) was added to a solution of 2-Piperidin-4-yl-1,2,4,10-tetrahydro-3-oxa-10-aza-phenanthren-9-one trifluoroacetate (60 mg, 151 μmol, Eq: 1.00) in NMP (1.00 mL). The mixture was allowed to stir at room temperature for 18 h. The resulting mixture was poured into 5 mL ethyl acetate and washed with 1 M NaOH (2×50 mL). The organics was dried on sodium sulfate and concentrated in vacuo to afford 2-(1-methanesulfonyl-piperidin-4-yl)-1,2,4,10-tetrahydro-3-oxa-10-aza-phenanthren-9-one as a white solid (50 mg, 91%). ¹H NMR (DMSO-d₆) δ: 11.25 (s, 1H), 8.19 (d, J=7.9 Hz, 1H), 7.56-7.78 (m, 1H), 7.41 (d, J=8.3 Hz, 1H), 7.45 (t, J=6.8 Hz, 1H), 4.91 (d, J=14.4 Hz, 1H), 4.61 (d, J=14.4 Hz, 1H), 3.60 (d, J=11.7 Hz, 2H), 3.50 (q, J=6.9 Hz, 1H), 2.59-2.77 (m, 3H), 2.02 (d, J=13.2 Hz, 1H), 1.65-1.77 (m, 1H), 1.54-1.64 (m, 1H), 1.18-1.46 (m, 2H). MS calcd. for C₁₈H₂₂N₂O₄S [(M+H)⁺] 363.5, obsd. 363.

### Example 26

### rac-(2-{2-[2-(2-{3-Oxo-3-[4-(9-oxo-1,4,9,10-tetrahydro-2H-3-oxa-10-aza-phenanthren-2-yl)-piperidin-1-yl]-propoxy}-ethoxy)-ethoxy]-ethoxy}-ethyl)-carbamic acid tert-butyl ester

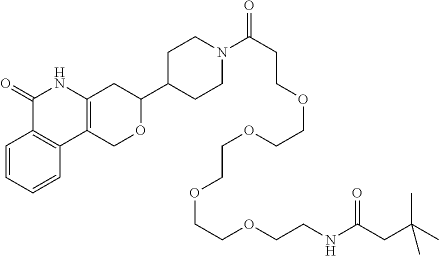 [SMILES: CC(C)(C)CC(=O)NCCOCCOCCOCCOCCC(=O)N1CCC(C2Cc3[nH]c(=O)c4ccccc4c3CO2)CC1]

A mixture of HBTU (74.3 mg, 0.196 mmol), Hunig's base (0.132 ml, 0.753 mmol), 2-piperidin-4-yl-1,2,4,10-tetrahydro-3-oxa-10-aza-phenanthren-9-one trifluoroacetate (60 mg, 0.151 mmol), 3-(2-{2-[2-(2-tert-butoxycarbonylamino-ethoxy)-ethoxy]-ethoxy}-ethoxy)-propionic acid (71.5 mg, 0.196 mmol,) in NMP (1.8 mL) was stirred at room temperature for 18 h. The resulting mixture was diluted with ethyl acetate, poured into 0.1 M sodium hydroxide, and extracted with ethyl acetate (2×50 mL). The organic layers were combined, washed with H₂O (2×50 mL), dried over MgSO₄, and concentrated in vacuo to afford (2-{2-[2-(2-{3-Oxo-3-[4-(9-oxo-1,4, 9,10-tetrahydro-2H-3-oxa-10-aza-phenanthren-2-yl)-piperidin-1-yl]-propoxy}-ethoxy)-ethoxy]-ethoxy}-ethyl)-carbamic acid tert-butyl ester as an off-white solid (50 mg, 52%). ¹H NMR (DMSO-d₆) δ: 11.24 (s, 1H), 8.18 (d, J=7.2 Hz, 1H), 7.60-7.78 (m, 1H), 7.40 (d, J=7.9 Hz, 1H), 7.45 (t, J=7.6 Hz, 1H), 6.74 (t, J=4.7 Hz, 1H), 4.90 (d, J=14.4 Hz, 1H), 4.59 (d, J=14.7 Hz, 1H), 4.43 (br. d, J=12.8 Hz, 1H), 3.93 (br. d, J=14.0 Hz, 1H), 3.61 (t, J=6.8 Hz, 2H), 3.33-3.38 (m, 2H), 3.03 (q, J=6.0 Hz, 2H), 2.85-2.99 (m, 1H), 2.56 (td, J=6.8, 2.3 Hz, 2H), 1.92 (br. t, J=12.8 Hz, 1H), 1.65-1.82 (m, 1H), 1.50-1.65 (m, 1H), 1.35 (s, 9H), 1.00-1.15 (m, 1H). MS calcd. for C₃₃H₄₉N₃O₉ [(M−H)^{-]} 630.8, obsd. 630.

### Example 27

### rac-6-Chloro-2-isopropyl-1,2,4,10-tetrahydro-3-oxa-10-aza-phenanthren-9-one (I-27)

6-Chloro-2-isopropyl-1,2,4,10-tetrahydro-3-oxa-10-aza-phenanthren-9-one was synthesized following the procedure in Example 7. From 2-isopropyl-tetrahydro-pyran-4-one and 2-bromo-4-chloro-benzoic acid methyl ester: 6-chloro-2-isopropyl-1,2,4,10-tetrahydro-3-oxa-10-aza-phenanthren-9-one was obtained as a white solid (375 mg, 32%). ¹H NMR (DMSO-d₆) δ: 11.35 (br. s., 1H), 8.16 (d, J=9.1 Hz, 1H), 7.32-7.54 (m, 2H), 4.89 (d, J=14.4 Hz, 1H), 4.55 (dt, J=14.4, 2.2 Hz, 1H), 3.32-3.41 (m, 1H), 1.76 (dq, J=13.6, 6.8 Hz, 1H), 0.97 (d, J=6.8 Hz, 3H), 0.92 (d, J=6.8 Hz, 3H). MS calcd. for C₁₅H₁₆ClNO₂ [(M+H)⁺] 278.7, obsd. 278.

### Example 28

### rac-7-Isopropyl-5,6,7,9-tetrahydro-8-oxa-1-thia-5-aza-cyclopenta[a]naphthalen-4-one (I-28)

7-Isopropyl-5,6,7,9-tetrahydro-8-oxa-1-thia-5-aza-cyclopenta[a]naphthalen-4-one was synthesized following the procedure in Example 7. From 2-isopropyl-tetrahydro-pyran-4-one 2-bromo-thiophene-3-carboxylic acid methyl ester: 7-isopropyl-5,6,7,9-tetrahydro-8-oxa-1-thia-5-aza-cyclopenta[a]naphthalen-4-one was obtained as an off-white solid (25 mg, 5%). ¹H NMR (DMSO-d₆) δ: 11.40 (br. s., 1H), 7.53 (d, J=5.7 Hz, 1H), 7.47 (d, J=5.7 Hz, 1H), 4.63 (d, J=14.0 Hz, 1H), 4.53 (dt, J=13.6, 2.1 Hz, 1H), 3.42 (ddd, J=9.1, 6.4, 4.9 Hz, 1H), 1.77 (dq, J=13.5, 6.8 Hz, 1H), 0.92 (d, J=6.8 Hz, 3H), 0.96 (d, J=6.8 Hz, 3H). MS calcd. for C₁₃H₁₅NO₂S [(M+H)⁺] 250.3, obsd. 250.

### Example 29

### rac-2-(2-Benzyloxy-1,1-dimethyl-ethyl)-1,2,4,10-tetrahydro-3-oxa-10-aza-phenanthren-9-one (I-29)

2-(2-Benzyloxy-1,1-dimethyl-ethyl)-1,2,4,10-tetrahydro-3-oxa-10-aza-phenanthren-9-one was synthesized following the procedure in Example 7. From 2-(2-benzyloxy-1,1-dimethyl-ethyl)-tetrahydro-pyran-4-one and methyl 2-bromobenzoate: 2-(2-benzyloxy-1,1-dimethyl-ethyl)-1,2,4,10-tetrahydro-3-oxa-10-aza-phenanthren-9-one was obtained as an off-white solid (620 mg, 30%). ¹H NMR (DMSO-d₆) δ: 11.19 (s, 1H), 8.18 (dd, J=8.3, 1.1 Hz, 1H), 7.68 (td, J=8.3, 1.5 Hz, 1H), 7.44 (t, J=7.2 Hz, 1H), 7.39 (d, J=8.3 Hz, 1H), 7.23-7.36 (m, 5H), 4.92 (d, J=14.4 Hz, 1H), 4.55 (d, J=15.5 Hz, 1H), 4.48 (d, J=3.0 Hz, 2H), 3.60 (dd, J=11.1, 3.2 Hz, 1H), 3.35 (d, J=8.7 Hz, 1H), 3.27 (d, J=8.7 Hz, 1H), 2.62 (dd, J=17.0, 11.0 Hz, 1H), 2.39 (d, J=16.6 Hz, 1H), 0.96 (s, 3H), 0.92 (s, 3H). MS calcd. for C₂₃H₂₅NO₃ [(M+H)⁺] 364.5, obsd. 364.

### Example 30

### rac-2-(2-Hydroxy-1,1-dimethyl-ethyl)-1,2,4,10-tetrahydro-3-oxa-10-aza-phenanthren-9-one (I-30)

A mixture of 2-(2-benzyloxy-1,1-dimethyl-ethyl)-1,2,4,10-tetrahydro-3-oxa-10-aza-phenanthren-9-one (100 mg, 0.275 mmol), Pearlman's catalyst (50 mg), and HCl (1 drop) in 1:1 ethanol/dioxane (10 mL) was placed under a hydrogen atmosphere (3 bars) and reacted at room temperature for 8 h. The mixture was filtered through a pad of celite and the filtrate concentrated in vacuo. The crude material was purified by column chromatography (SiO₂, 0% to 100% ethyl acetate in heptanes) to afford 2-(2-hydroxy-1,1-dimethyl-ethyl)-1,2,4,10-tetrahydro-3-oxa-10-aza-phenanthren-9-one as off-white solid (18 mg, 24%). ¹H NMR (DMSO-d₆) δ: 11.20 (s, 1H), 8.18 (dd, J=7.8, 1.1 Hz, 1H), 7.68 (td, J=7.8, 1.0 Hz, 1H), 7.39 (d, J=7.9 Hz, 1H), 7.44 (t, J=7.6 Hz, 1H), 4.91 (d, J=14.0 Hz, 1H), 4.57 (t, J=5.3 Hz, 1H), 4.58 (d, J=14.0 Hz, 1H), 3.54 (dd, J=11.0, 3.0 Hz, 1H), 3.20-3.27 (m, 1H), 2.53-2.66 (m, 1H), 2.33-2.43 (m, 1H), 0.91 (s, 3H), 0.83 (s, 3H).

### Example 31

### rac-7-Cyclopentyl-5,7,8,9-tetrahydro-6-oxa-3,9-diaza-phenanthren-10-one (I-31)

Methyl 3-bromoisonicotinate (0.4 g, 1.85 mmol, Eq: 1.00), 2-cyclopentyldihydro-2H-pyran-4(3H)-one (374 mg, 2.22 mmol, Eq: 1.2), Pd₂(dba)₃ (0) (33.9 mg, 37.0 μmol, Eq: 0.02), xantphos (42.9 mg, 74.1 μmol, Eq: 0.04) and Cs₂CO₃ (800 mg, 2.46 mmol, Eq: 1.33) were placed in a microwave vial. Under N₂, toluene (10 mL) was added. The resulting mixture stirred in the microwave (Discover CEM) at 130° C. for 50 minutes. Fresh Pd₂(dpa)₃ (33.9 mg, 37.0 μmol, Eq: 0.02) and xantphos (42.9 mg, 74.1 μmol, Eq: 0.04) were added and the mixture stirred in the microwave (discover CEM) at 130° C. for 60 minutes. The solvent was pipetted off onto a column for purification by flash chromatography (40 g SiO₂, hexanes/EtOAc 0-40% EtOAc) to give 7-cyclopentyl-7,8-dihydro-5H-6,9-dioxa-3-aza-phenanthren-10-one as light yellow solid (103 mg, 20% yield). ¹H NMR (CDCl₃) δ: 8.81 (d, J=5.3 Hz, 1H), 8.73 (s, 1H), 8.08-8.16 (m, 1H), 4.99 (d, J=14.1 Hz, 1H), 4.75 (dt, J=14.2, 2.8 Hz, 1H), 3.53-3.61 (m, 1H), 2.56-2.73 (m, 2H), 2.06-2.18 (m, 1H), 1.89-2.00 (m, 1H), 1.76-1.87 (m, 1H), 1.57-1.75 (m, 5H), 1.46-1.56 (m, 1H), 1.26-1.38 (m, 1H). MS calcd. for C₁₆H₁₇NO₃ [(M+H)⁺] 272.3, obsd. 272.

7-Cyclopentyl-7,8-dihydro-5H-6,9-dioxa-3-aza-phenanthren-10-one (0.1 g, 369 μmol, Eq: 1.00) stirred in ammonia in MeOH ca. 7M (2.36 g, 3 ml, 139 mmol, Eq: 376) in the microwave (Biotage) at 140° C. for 1 h. The precipitate was filtered off, washed with a small volume of MeOH and was dried to give 7-cyclopentyl-5,7,8,9-tetrahydro-6-oxa-3,9-diaza-phenanthren-10-one as on off-white solid (60 mg, 60% yield). ¹H NMR (DMSO-d₆) δ: 8.90 (s, 1H), 8.64 (d, J=5.3 Hz, 1H), 8.00 (d, J=5.3 Hz, 1H), 5.06 (d, J=14.3 Hz, 1H), 4.71 (d, J=14.6 Hz, 1H), 3.44-3.53 (m, 1H), 2.02 (sxt, J=8.0 Hz, 1H), 1.76-1.87 (m, 1H), 1.39-1.75 (m, 6H), 1.25-1.37 (m, 1H). MS calcd. for C₁₆H₁₈N₂O₂ [(M+H)⁺] 271.3, obsd. 271.

### Example 32

### rac-4-(9-Oxo-1,4,9,10-tetrahydro-2H-3-oxa-10-aza-phenanthren-2-yl)-piperidine-1-carboxylic acid isopropyl ester (I-32)

In a 2 mL vial, 3-(piperidin-4-yl)-3,4-dihydro-1H-pyrano[4,3-c]isoquinolin-6(5H)-one (20 mg, 70.3 μmol, Eq: 1.00) was combined with DMF (0.7 mL) to give a light brown solution. DIPEA (27.3 mg, 36.9 μL, 211 μmol, Eq: 3.00) was added. Isopropyl chloroformate (1M in toluene) (106 μL, 106 μmol, Eq: 1.50) was added. The mixture was stirred at RT for 1 hr. The mixture was precipitated with water, filtered, washed filter cake with EtOAc and MeOH, combined and evaporated. The product was dried under vacuum to yield 9 mg (35%) of 4-(9-oxo-1,4,9,10-tetrahydro-2H-3-oxa-10-aza-phenanthren-2-yl)-piperidine-1-carboxylic acid isopropyl ester as a light brown solid. ¹H NMR (DMSO-d₆) δ: 11.25 (br. s., 1H), 8.20 (dd, J=7.9, 1.1 Hz, 1H), 7.64 (td, J=8.0, 1.0 Hz, 1H), 7.42 (t, J=7.7 Hz, 1H), 7.38 (d, J=8.3 Hz, 1H), 4.85 (m, 2H), 4.59 (d, J=14.7 Hz, 1H), 3.98 (d, J=12.8 Hz, 2H), 3.39-3.52 (m, 1H), 2.69 (m, 2H), 1.90 (d, J=15.1 Hz, 1H), 1.68 (m, 2H), 1.57 (m, 2H), 1.34 (d, J=6 Hz, 6H), 1.06-1.26 (m, 2H). MS calcd. for C₂₁H₂₆N₂O₄ [(M+H)⁺] 371.5, obsd. 371.1.

### Example 33

### rac-2-(1-Cyclopropanecarbonyl-piperidin-4-yl)-1,2,4,10-tetrahydro-3-oxa-10-aza-phenanthren-9-one (I-33)

In a 2 mL vial, 3-(piperidin-4-yl)-3,4-dihydro-1H-pyrano[4,3-c]isoquinolin-6(5H)-one (20 mg, 70.3 μmol, Eq: 1.00) was combined with DMF (700 μL) to give a light brown solution. DIPEA (27.3 mg, 36.9 μL, 211 μmol, Eq: 3.00) was added. Cyclopropanecarbonyl chloride (11.0 mg, 9.57 μL, 106 μmol, Eq: 1.50) was added. The mixture was stirred at RT for 1 hr. The mixture was precipitated with H₂O, washed with 0.1 N HCl, water and dried under vacuum to give 13 mg (52%) of 2-(1-cyclopropanecarbonyl-piperidin-4-yl)-1,2,4,10-tetrahydro-3-oxa-10-aza-phenanthren-9-one as a light brown solid. ¹H NMR (DMSO-d₆) δ: 11.25 (br. s., 1H), 8.19 (d, J=7.9 Hz, 1H), 7.61-7.75 (m, 1H), 7.45 (t, J=7.9 Hz, 1H), 7.41 (d, J=8.3 Hz, 1H), 4.80-5.01 (m, J=3.8 Hz, 1H), 4.64 (d, J=14.4 Hz, 1H), 3.72-3.96 (m, 1H), 3.36-3.72 (m, 3H), 2.97-3.26 (m, 1H), 2.55 (d, J=6.0 Hz, 1H), 2.26-2.42 (m, 1H), 1.95-2.26 (m, 2H), 1.82-1.95 (m, 2H), 1.53-1.82 (m, 2H), 0.62-0.80 (m, 4H). MS calcd. for C₂₁H₂₄N₂O₃ [(M+H)⁺] 353.4, obsd. 353.1.

### Example 34

### rac-7-tert-Butyl-4a,5,7,8,9,10a-hexahydro-6-oxa-3,9-diaza-phenanthren-10-one (I-34)

But-3-en-1-ol (1.98 g, 2 ml, 27.5 mmol, Eq: 1.00) and pivalaldehyde (4.74 g, 5.98 ml, 55.0 mmol, Eq: 2) stirred in dichloromethane (150 mL) at 0° C. TFA (44.4 g, 30 ml, 389 mmol, Eq: 14.2) was added via addition funnel over 15 minutes. After the addition was complete, the ice bath was removed and the clear light brown solution stirred at RT overnight. The solvent was mostly removed under reduced pressure. The residue was taken up in toluene and concentrated again. The remaining oil was taken up in methanol (100 mL) and was poured onto Na₂CO₃ (25 g, 236 mmol, Eq: 8.57) and stirred overnight. The mixture was filtered through Celite and the filtrate was concentrated to dryness. The residue was partitioned between water and DCM. The aqueous layer was washed with DCM, the organic layers were extracted with brine, combined, dried over Na₂SO₄, filtered and concentrated to give 2-tert-butyltetrahydro-2H-pyran-4-ol as light yellow oil (3.1 g, 71% yield). ¹H NMR (DMSO-d₆) δ: 4.74 (br. s., 1H), 3.89 (ddd, J=11.5, 4.8, 1.3 Hz, 1H), 3.54 (br. s., 1H), 3.25 (s, 1H), 3.18 (d, J=3.3 Hz, 1H), 2.83 (dd, J=11.3, 1.5 Hz, 1H), 1.74-1.82 (m, 1H), 1.65-1.74 (m, 1H), 1.17-1.30 (m, 1H), 0.95-1.06 (m, 1H), 0.85 (s, 9H).

2-tert-butyltetrahydro-2H-pyran-4-ol (3.09 g, 19.5 mmol, Eq: 1.00), Silica gel (15 g, 19.5 mmol, Eq: 1.00) and PCC (6.31 g, 29.3 mmol, Eq: 1.5) stirred in dichloromethane (100 mL) at RT overnight. The reaction mixture was filtered through Celite and the filtrate was concentrated. A biphasic oily mixture was obtained; the lighter, liquid top layer was pipetted to give 2-tert-butyldihydro-2H-pyran-4(3H)-one as brown liquid (2.9 g, 95% yield). ¹H NMR (CDCl₃) δ: 4.29-4.39 (m, 1H), 3.57-3.68 (m, 1H), 3.21 (dd, J=11.5, 2.8 Hz, 1H), 2.53-2.66 (m, 1H), 2.28-2.45 (m, 3H), 0.96 (s, 9H).

Methyl 3-bromoisonicotinate (0.4 g, 1.85 mmol, Eq: 1.00), 2-tert-butyldihydro-2H-pyran-4(3H)-one (347 mg, 2.22 mmol, Eq: 1.2), Pd₂(dba)₃ (0) (33.9 mg, 37.0 μmol, Eq: 0.02), xantphos (42.9 mg, 74.1 μmol, Eq: 0.04) and Cs₂CO₃ (800 mg, 2.46 mmol, Eq: 1.33) were placed in a microwave vial. Under N₂, toluene (10.0 mL) was added. The resulting mixture stirred in the microwave (Biotage Initiator) at 135° C. for 80 minutes. The solvent was pipetted off onto a column for purification by flash chromatography (40 g SiO₂, hexanes/EtOAc 0-40% EtOAc). The combined fractions were concentrated and the remaining yellow solid was triturated in hexanes to give 7-tert-butyl-4a,7,8,10a-tetrahydro-5H-6,9-dioxa-3-aza-phenanthren-10-one as light yellow solid (125 mg, 26% yield). ¹H NMR (CDCl₃) δ: 8.80 (d, J=5.0 Hz, 1H), 8.74 (s, 1H), 8.16 (d, J=5.3 Hz, 1H), 5.01 (dd, J=14.1, 1.5 Hz, 1H), 4.72 (dt, J=14.1, 2.9 Hz, 1H), 3.43 (dd, J=11.0, 3.3 Hz, 1H), 2.67-2.80 (m, 1H), 2.49 (dt, J=17.4, 2.7 Hz, 1H), 1.04 (s, 9H). MS calcd. for C₁₅H₁₇NO₃ [(M+H)⁺] 260.3, obsd. 260.

7-tert-Butyl-4a,7,8,10a-tetrahydro-5H-6,9-dioxa-3-aza-phenanthren-10-one (0.12 g, 463 μmol, Eq: 1.00) was suspended in ammonia in MeOH ca. 7M (2.36 g, 3 ml, 139 mmol, Eq: 300) and stirred in the microwave (Biotage Initiator) at 140° C. for 1 h. The precipitate was filtered off, washed with a small volume of MeOH and was dried to give 7-tert-butyl-4a,5,7,8,9,10a-hexahydro-6-oxa-3,9-diaza-phenanthren-10-one as light yellow crystalline solid (117 mg, 97% yield). ¹H NMR (DMSO-d₆) δ: 8.90 (s, 1H), 8.64 (d, J=5.3 Hz, 1H), 8.00 (d, J=5.0 Hz, 1H), 5.12 (d, J=14.3 Hz, 1H), 4.70 (d, J=14.3 Hz, 1H), 3.38 (dd, J=10.8, 3.3 Hz, 1H), 2.53-2.62 (m, 1H), 2.40-2.49 (m, 1H), 0.97 (s, 9H). MS calcd. for C₁₅H₁₈N₂O₂ [(M+H)⁺] 259.3, obsd. 259.

### Example 35

### 3-(9-Oxo-1,4,9,10-tetrahydro-2H-3-oxa-10-aza-phenanthren-2-yl)-pyrrolidine-1-carboxylic acid tert-butyl ester (I-35)

3-(9-Oxo-1,4,9,10-tetrahydro-2H-3-oxa-10-aza-phenanthren-2-yl)-pyrrolidine-1-carboxylic acid tert-butyl ester was synthesized following the procedure in Example 7. From 3-(4-oxo-tetrahydro-pyran-2-yl)-pyrrolidine-1-carboxylic acid tert-butyl ester and methyl 2-bromobenzoate: 3-(9-oxo-1,4,9,10-tetrahydro-2H-3-oxa-10-aza-phenanthren-2-yl)-pyrrolidine-1-carboxylic acid tert-butyl ester was obtained as an off-white solid (721 mg, 40%). ¹H NMR (DMSO-d₆) δ: 11.24 (s, 1H), 8.18 (dd, J=7.9, 1.1 Hz, 1H), 7.69 (t, J=7.9 Hz, 1H), 7.40 (d, J=7.9 Hz, 1H), 7.45 (t, J=7.7 Hz, 1H), 4.90 (dd, J=14.4, 4.5 Hz, 1H), 4.63 (d, J=14.7 Hz, 1H), 3.44-3.68 (m, 2H), 3.27-3.44 (m, 2H), 2.98-3.26 (m, 2H), 2.30-2.43 (m, 1H), 1.82-2.12 (m, 1H), 1.54-1.77 (m, 1H), 1.39 (s, 9H).

### Example 36

### 2-Pyrrolidin-3-yl-1,2,4,10-tetrahydro-3-oxa-10-aza-phenanthren-9-one, hydrochloride (I-36)

From 3-(9-oxo-1,4,9,10-tetrahydro-2H-3-oxa-10-aza-phenanthren-2-yl)-pyrrolidine-1-carboxylic acid tert-butyl ester: 2-pyrrolidin-3-yl-1,2,4,10-tetrahydro-3-oxa-10-aza-phenanthren-9-one hydrochloride was obtained as a yellow solid (123 mg, 99%): MS calcd. for C₁₆H₁₈N₂O₂ [(M+H)⁺] 271.3, obsd. 271.

### Example 37

### 2-(1-Acetyl-pyrrolidin-3-yl)-1,2,4,10-tetrahydro-3-oxa-10-aza-phenanthren-9-one (I-37)

2-(1-Acetyl-pyrrolidin-3-yl)-1,2,4,10-tetrahydro-3-oxa-10-aza-phenanthren-9-one was synthesized following the procedure in Example 24. From 2-pyrrolidin-3-yl-1,2,4,10-tetrahydro-3-oxa-10-aza-phenanthren-9-one hydrochloride and acetyl chloride: 2-(1-acetyl-pyrrolidin-3-yl)-1,2,4,10-tetrahydro-3-oxa-10-aza-phenanthren-9-one was obtained as an off-white solid (35 mg, 38%). ¹H NMR (DMSO-d₆) δ: 11.25 (d, J=3.4 Hz, 1H), 8.19 (d, J=7.9 Hz, 1H), 7.64-7.84 (m, 1H), 7.45 (t, J=7.7 Hz, 1H), 7.41 (d, J=7.9 Hz, 1H), 4.90 (d, J=14.4 Hz, 1H), 4.64 (dd, J=14.4, 1.9 Hz, 1H), 3.52-3.76 (m, 3H), 3.36-3.52 (m, 1H), 2.97-3.24 (m, 1H), 2.21-2.41 (m, 1H), 1.81-2.05 (m, 1H), 1.93 (d, J=7.6 Hz, 5H), 1.46-1.81 (m, 1H). MS calcd. for C₁₈H₂₀N₂O₃ [(M+H)⁺] 313.4, obsd. 313.

### Example 38

### rac-6-Cyclopropyl-2-isopropyl-1,2,4,10-tetrahydro-3-oxa-10-aza-phenanthren-9-one (I-38)

A mixture of 6-chloro-2-isopropyl-1,2,4,10-tetrahydro-3-oxa-10-aza-phenanthren-9-one (100 mg, 0.36 mmol), cyclopropylboronic acid (55.7 mg, 0.648 mmol), palladium (II) acetate (4.04 mg, 0.018 mmol), tricyclohexylphosphine (10.1 mg, 0.036 mmol), and potassium phosphate tribasic (153 mg, 0.720 mmol) suspended in dioxane and water was placed under argon and heated at 100° C. overnight. The resulting mixture was cooled to room temperature and then extracted with ethyl acetate (3×100 mL). The combined organic layers were washed with water (20 mL), brine (20 mL), dried on sodium sulfate, and concentrated in vacuo. The residue was pre-absorbed onto silica and purified by flash chromatography (SiO₂, 0% to 10% EtOAc in methylene chloride) to afford 6-cyclopropyl-2-isopropyl-1,2,4,10-tetrahydro-3-oxa-10-aza-phenanthren-9-one as a white solid (69 mg, 68%). ¹H NMR (DMSO-d₆) δ: 11.06 (br. s., 1H), 8.04 (d, J=9.1 Hz, 1H), 7.07 (d, J=7.9 Hz, 1H), 7.06 (s, 1H), 4.90 (d, J=14.4 Hz, 1H), 4.57 (d, J=14.4 Hz, 1H), 3.35-3.40 (1H, m), 2.43 (d, J=6.4 Hz, 2H), 1.96-2.10 (m, 1H), 1.75 (dq, J=13.5, 6.7 Hz, 1H), 0.99-1.07 (m, 2H), 0.98 (d, J=7.2 Hz, 2H), 0.92 (d, J=6.8 Hz, 1H), 0.75-0.84 (m, 2H). MS calcd. for C₁₈H₂₁NO₂ [(M+H)⁺] 284.4, obsd. 284.1.

### Example 39

### 2-(1-Methanesulfonyl-pyrrolidin-3-yl)-1,2,4,10-tetrahydro-3-oxa-10-aza-phenanthren-9-one (I-39)

2-(1-Methanesulfonyl-pyrrolidin-3-yl)-1,2,4,10-tetrahydro-3-oxa-10-aza-phenanthren-9-one was synthesized following the procedure in Example 25. From 2-pyrrolidin-3-yl-1,2,4,10-tetrahydro-3-oxa-10-aza-phenanthren-9-one hydrochloride and methanesulfonyl chloride: 241-methanesulfonyl-pyrrolidin-3-yl)-1,2,4,10-tetrahydro-3-oxa-10-aza-phenanthren-9-one was obtained as a white solid (50 mg, 43%). ¹H NMR (DMSO-d₆) δ: 11.26 (d, J=4.5 Hz, 1H), 8.19 (dd, J=8.3, 1.1 Hz, 1H), 7.69 (td, J=7.6, 1.5 Hz, 1H), 7.41 (d, J=7.9 Hz, 1H), 7.45 (t, J=7.6 Hz, 1H), 4.91 (dd, J=14.2, 4.0 Hz, 1H), 4.64 (d, J=14.4 Hz, 1H), 3.53-3.74 (m, 1H), 3.46 (dd, J=10.2, 7.9 Hz, 1H), 3.01-3.28 (m, 2H), 2.90 (d, J=3.0 Hz, 3H), 1.67-2.22 (m, 2H). MS calcd. for C₁₇H₂₀N₂O₄S [(M+H)⁺] 349.4, obsd. 349.0.

### Example 40

### rac-7-tert-Butyl-5,6,7,9-tetrahydro-8-oxa-3-thia-5-aza-cyclopenta[a]naphthalen-4-one (I-40)

Methyl 3-bromothiophene-2-carboxylate (0.2 g, 905 mmol, Eq: 1.00), 2-tert-butyldihydro-2H-pyran-4(3H)-one (170 mg, 1.09 mmol, Eq: 1.2), Pd₂(dba)₃ (0) (16.6 mg, 18.1 μmol, Eq: 0.02), xantphos (20.9 mg, 36.2 μmol, Eq: 0.04) and Cs₂CO₃ (400 mg, 1.23 mmol, Eq: 1.36) were placed in a microwave vial. Under N₂, toluene (5 mL) was added. The resulting mixture stirred in the microwave (Biotage Initiator) at 100° C. for 50 minutes. Fresh tris(dibenzylideneacetone)-dipalladium (0) (16.6 mg, 18.1 μmol, Eq: 0.02) and xantphos (20.9 mg, 36.2 μmol, Eq: 0.04) were added and the mixture stirred in the microwave (Biotage Initiator) at 135° C. for 70 minutes. The solvent was pipetted off onto a column for purification by flash chromatography (24 g SiO₂, hexanes/EtOAc 0-17% EtOAc). Product fractions were combined and concentrated. The residue was triturated in hexanes. The solid was filtered off and dried to give 7-tert-butyl-6,9-dihydro-7H-5,8-dioxa-3-thia-cyclopenta[a]naphthalen-4-one as off-white crystalline solid (0.07 g, 265 mmol, 29.3% yield). MS calcd. for C₁₄H₁₆O₃S [(M+H)⁺] 265.4, obsd. 265.

7-tert-Butyl-6,9-dihydro-7H-5,8-dioxa-3-thia-cyclopenta[a]naphthalen-4-one (0.067 g, 253 mmol, Eq: 1.00) stirred in ammonia in MeOH ˜7M (1.18 g, 1.5 ml, 69.3 mmol, Eq: 273) in the microwave (Biotage Initiator) at 140° C. for 1 h. The precipitate was filtered off, washed with a small volume of MeOH and dried to give 7-tert-butyl-5,6,7,9-tetrahydro-8-oxa-3-thia-5-aza-cyclopenta[a]naphthalen-4-one as white crystalline solid (0.043 g, 162 μmol, 63.8% yield). ¹H NMR (Acetone-d₆) δ: 7.98 (d, J=5.3 Hz, 1H), 7.27 (d, J=5.3 Hz, 1H), 4.94 (dd, J=14.1, 1.3 Hz, 1H), 4.72 (dt, J=14.1, 2.5 Hz, 1H), 3.44 (dd, J=10.7, 3.6 Hz, 1H), 2.57-2.77 (m, 2H), 1.02 (s, 9H). MS calcd. for C₁₄H₁₇NO₂S [(M+H)⁺] 264.4, obsd. 264.

### Example 41

### rac-2-tert-Butyl-5-methyl-1,2,4,10-tetrahydro-3-oxa-10-aza-phenanthren-9-one (I-41)

Methyl 2-bromo-3-methylbenzoate (0.2 g, 873 μmol, Eq: 1.00), 2-tert-butyl-dihydro-2H-pyran-4(3H)-one (164 mg, 1.05 mmol, Eq: 1.2), Pd₂(dba)₃ (16.0 mg, 17.5 μmol, Eq: 0.02), xantphos (20.2 mg, 34.9 μmol, Eq: 0.04) and Cs₂CO₃ (400 mg, 1.23 mmol, Eq: 1.41) were placed in a microwave vial. Under N₂, toluene (5 mL) was added. The resulting mixture stirred in the microwave (Biotage Initiator) at 100° C. for 50 minutes. Fresh tris(dibenzylideneacetone)-dipalladium (0) (16.0 mg, 17.5 μmol, Eq: 0.02) and xantphos (20.2 mg, 34.9 μmol, Eq: 0.04) were added and the mixture stirred in the microwave (Biotage) at 135° C. for 70 minutes. The solvent was pipetted off onto a column for purification by flash chromatography (24 g SiO₂, hexanes/EtOAc 0-15% EtOAc). Fractions were combined and concentrated. The yellow residue was triturated in hexanes. The solid was filtered off and dried to give 3-tert-butyl-10-methyl-3,4-dihydropyrano[4,3-c]isochromen-6(1H)-one as light yellow crystalline solid (15 mg, 6% yield). ¹H NMR (CDCl₃) δ: 8.27 (d, J=7.5 Hz, 1H), 7.51 (d, J=7.3 Hz, 1H), 7.34-7.42 (m, 1H), 5.14 (dd, J=14.1, 1.3 Hz, 1H), 4.88 (dt, J=13.9, 3.0 Hz, 1H), 3.37 (dd, J=11.2, 3.4 Hz, 1H), 2.67-2.80 (m, 1H), 2.59 (s, 3H), 2.49 (dt, J=17.3, 2.9 Hz, 1H), 1.02 (s, 9H). MS calcd. for C₁₇H₂₀O₃ [(M+H)⁺] 273.4, obsd. 273.

3-tert-Butyl-10-methyl-3,4-dihydropyrano[4,3-c]isochromen-6(111)-one (0.013 g, 47.7 μmol, Eq: 1.00) stirred in ammonia in MeOH ˜7M (472 mg, 0.6 ml, 27.7 mmol, Eq: 581) in the microwave (Biotage Initiator) at 140° C. for 1 h. The solvent was removed. The remaining solid was triturated in little MeOH, filtered and dried to give 2-tert-butyl-5-methyl-1,2,4,10-tetrahydro-3-oxa-10-aza-phenanthren-9-one as an off-white solid (9 mg, 32.8 μmol, 68.8% yield). ¹H NMR (Acetone) δ: 8.25 (d, J=7.5 Hz, 1H), 7.49 (d, J=7.0 Hz, 1H), 7.32 (t, J=7.5 Hz, 1H), 5.20 (dd, J=13.8, 1.0 Hz, 1H), 4.99 (dt, J=13.8, 2.6 Hz, 1H), 3.40 (dd, J=11.2, 3.6 Hz, 1H), 2.70-2.79 (m, 1H), 2.67 (s, 3H), 2.53-2.63 (m, 1H), 1.02 (s, 9H). MS calcd. for C₁₇H₂₁NO₂ [(M+H)⁺] 272.4, obsd. 272.

### Example 42

### rac-7-tert-Butyl-1-methyl-5,6,7,9-tetrahydro-1H-8-oxa-1,2,5-triaza-cyclopenta[a]naphthalen-4-one (I-42)

7-tert-Butyl-1-methyl-5,6,7,9-tetrahydro-1H-8-oxa-1,2,5-triaza-cyclopenta[a]naphthalen-4-one was synthesized following the procedure in Example 7. From 2-tert-butyl-tetrahydro-pyran-4-one and 5-bromo-1H-pyrazole-4-carboxylic acid ethyl ester: 7-tert-butyl-1-methyl-5,6,7,9-tetrahydro-1H-8-oxa-1,2,5-triaza-cyclopenta[a]naphthalen-4-one was obtained as a white solid (25 mg, 2%). ¹H NMR (DMSO-d₆) δ: 10.88 (s, 1H), 7.89 (s, 1H), 5.18 (d, J=14.0 Hz, 1H), 4.81 (d, J=14.0 Hz, 1H), 3.98 (s, 3H), 0.93 (s, 9H). MS calcd. for C₁₄H₁₉N₃O₂ [(M+H)⁺] 262.3, obsd. 262.

### Example 43

### rac-7-tert-Butyl-5,7,8,9-tetrahydro-6-oxa-2,9-diaza-phenanthren-10-one (I-43)

7-tert-Butyl-5,7,8,9-tetrahydro-6-oxa-2,9-diaza-phenanthren-10-one was synthesized following the procedure in Example 7. From 2-tert-butyldihydro-2H-pyran-4(3H)-one, 4-bromo-nicotinic acid methyl ester, and potassium phosphate: 7-tert-butyl-5,7,8,9-tetrahydro-6-oxa-2,9-diaza-phenanthren-10-one was obtained as a grey solid (12 mg, 0.4%). ¹H NMR (DMSO-d₆) δ: 11.56 (br. s., 1H), 9.27 (s, 1H), 8.68 (d, J=5.7 Hz, 1H), 7.33 (d, J=5.3 Hz, 1H), 4.92 (d, J=14.4 Hz, 1H), 4.55 (dt, J=14.4, 2.2 Hz, 1H), 0.93 (s, 9H). MS calcd. for C₁₅H₁₈N₂O₂ [(M+H)⁺] 259.3, obsd. 259.

### Example 44

### 2-(1-Cyclopropanecarbonyl-pyrrolidin-3-yl)-1,2,4,10-tetrahydro-3-oxa-10-aza-phenanthren-9-one (I-44)

2-(1-Cyclopropanecarbonyl-pyrrolidin-3-yl)-1,2,4,10-tetrahydro-3-oxa-10-aza-phenanthren-9-one was synthesized following the procedure in Example 24. From 2-pyrrolidin-3-yl-1,2,4,10-tetrahydro-3-oxa-10-aza-phenanthren-9-one hydrochloride and cyclopropanecarbonyl chloride: 2-(1-cyclopropanecarbonyl-pyrrolidin-3-yl)-1,2,4,10-tetrahydro-3-oxa-10-aza-phenanthren-9-one was obtained as an off-white solid (76 mg, 69%). ¹H NMR (DMSO-d₆) δ: 11.25 (br. s., 1H), 8.19 (d, J=7.9 Hz, 1H), 7.61-7.75 (m, 1H), 7.45 (t, J=7.9 Hz, 1H), 7.41 (d, J=8.3 Hz, 1H), 4.80-5.01 (m, J=3.8 Hz, 1H), 4.64 (d, J=14.4 Hz, 1H), 3.72-3.96 (m, 1H), 3.36-3.72 (m, 3H), 2.97-3.26 (m, 1H), 2.55 (d, J=6.0 Hz, 1H), 2.26-2.42 (m, 1H), 1.95-2.26 (m, 1H), 1.82-1.95 (m, 1H), 1.53-1.82 (m, 2H), 0.62-0.80 (m, 4H). MS calcd. for C₂₀H₂₂N₂O₃ [(M+H)⁺] 339.4, obsd. 339.

### Example 45

### 2-[1-(1-Methyl-cyclopropanecarbonyl)-pyrrolidin-3-yl]-1,2,4,10-tetrahydro-3-oxa-10-aza-phenanthren-9-one (I-45)

2-[1-(1-Methyl-cyclopropanecarbonyl)-pyrrolidin-3-yl]-1,2,4,10-tetrahydro-3-oxa-10-aza-phenanthren-9-one was synthesized following the procedure in Example 26. From 2-pyrrolidin-3-yl-1,2,4,10-tetrahydro-3-oxa-10-aza-phenanthren-9-one hydrochloride and 1-methyl-cyclopropanecarboxylic acid: 2-[1-(1-methyl-cyclopropanecarbonyl)-pyrrolidin-3-yl]-1,2,4,10-tetrahydro-3-oxa-10-aza-phenanthren-9-one was obtained as an off-white solid (21 mg, 17%). ¹H NMR (DMSO-d₆) δ: 11.25 (br. s, 1H), 8.19 (d, J=7.2 Hz, 1H), 7.69 (t, J=7.0 Hz, 1H), 7.45 (t, J=7.6 Hz, 1H), 7.41 (d, J=7.9 Hz, 1H), 4.92 (d, J=14.4 Hz, 1H), 4.64 (d, J=13.6 Hz, 1H), 3.70-3.97 (m, 1H), 3.59 (td, J=9.0, 4.3 Hz, 1H), 3.38-3.69 (m, 1H), 2.28-2.42 (m, 1H), 2.00-2.20 (m, 1H), 1.84-2.00 (m, 1H), 1.56-1.84 (m, 1H), 1.23 (s, 3H), 0.80-0.94 (m, 1H), 0.67-0.80 (m, 1H), 0.34-0.59 (m, 2H). MS calcd. for C₂₁H₂₄N₂O₃ [(M+H)⁺] 353.4, obsd. 353.

### Example 46

### rac-2-[1-(1-Methyl-cyclopropanecarbonyl)-piperidin-4-yl]-1,2,4,10-tetrahydro-3-oxa-10-aza-phenanthren-9-one (I-46)

In a 5 mL round-bottomed flask, 4-(6-oxo-3,4,5,6-tetrahydro-1H-pyrano[4,3-c]isoquinolin-3-yl)piperidinium (31.4 mg, 110 mmol, Eq: 1.00), DIPEA (42.7 mg, 57.7 μL, 330 μmol, Eq: 3.00) and 1-methylcyclopropane-1-carboxylic acid (16.5 mg, 165 mmol, Eq: 1.50) were combined with DMF (1 mL) to give an off-white suspension. BOP (73.0 mg, 165 μmol, Eq: 1.50) was added. The mixture was stirred at RT for 1 hr. Additional DIPEA (42.7 mg, 57.7 μL, 330 μmol, Eq: 3.00) was added. The cloudy suspension immediately cleared and stirred for 3 hr. The reaction mixture was diluted with 50 mL EtOAc and washed with 2×5 mL 0.5 N HCl, 2×5 mL sat NaHCO₃, 2×5 mL sat NaCl, dried over MgSO₄, filtered and evaporated to 83.5 mg of a white solid. The crude material (adsorbed onto silica) was purified by flash chromatography (silica gel, 15×12 g, 0% to 100%, 5% MeOH/EtOAc in hexanes). Fractions were pooled and evaporated to yield 34.3 mg (85%) of 2-[1-(1-methyl-cyclopropanecarbonyl)-piperidin-4-yl]-1,2,4,10-tetrahydro-3-oxa-10-aza-phenanthren-9-one as a white solid. ¹H NMR (MeOH-d4) δ: 11.25 (br. s., 1H), 8.19 (d, J=7.9 Hz, 1H), 7.61-7.75 (m, 1H), 7.52 (t, J=7.9 Hz, 1H), 7.49 (d, J=8.3 Hz, 1H), 4.80-5.01 (m, J=3.8 Hz, 1H), 4.64 (d, J=14.4 Hz, 1H), 3.72-3.96 (m, 1H), 3.36-3.72 (m, 3H), 2.97-3.26 (m, 1H), 2.55 (d, J=6.0 Hz, 1H), 2.26-2.42 (m, 1H), 1.95-2.26 (m, 2H), 1.82-1.95 (m, 2H), 1.53-1.82 (m, 2H), 1.42 (s, 3H), 0.90 (m, 2H), 0.65 (m, 2H). MS calcd. for C₂₂H₂₆N₂O₃ [(M+H)⁺] 367.5, obsd. 367.0.

### Example 47

### rac-2-(Tetrahydro-pyran-4-yl)-1,2,4,10-tetrahydro-3-oxa-10-aza-phenanthren-9-one (I-47)

But-3-en-1-ol (992 mg, 1 ml, 13.8 μmol, Eq: 1.00) and tetrahydro-2H-pyran-4-carbaldehyde (3.6 g, 3.28 ml, 30.6 mmol, Eq: 2.22) stirred in dichloromethane (70 mL) at 0° C. TFA (22.2 g, 15 ml, 195 mmol, Eq: 14.2) was added via addition funnel over 15 minutes. After the addition was complete, the ice bath was removed and the mixture stirred at RT for 3 days. The solvent was mostly removed under reduced pressure. The residue was taken up in toluene and concentrated again. The remaining oil was taken up in methanol (50 mL) and was poured onto Na₂CO₃ (15 g, 142 mmol, Eq: 10.3) and stirred for ˜3 h. The mixture was filtered through Celite and the filtrate was concentrated to dryness. The residue was partitioned between water and DCM. The aqueous layer was washed with DCM, the organic layers were combined, dried over Na₂SO₄, filtered and concentrated to give octahydro-2H,2′H-2,4′-bipyran-4-ol as light brown crystalline solid (2.06 g, 80% yield).

Octahydro-2H,2H-2,4′-bipyran-4-ol (2.06 g, 11.1 mmol, Eq: 1.00), silica gel (20 g, 11.1 mmol, Eq: 1.00) and PCC (3.58 g, 16.6 mmol, Eq: 1.5) stirred in DCM (70 mL) at RT overnight. The mixture was filtered through Celite. The filtercake was washed with DCM and the filtrate was concentrated to dryness. The remaining dark brown solid was taken up in ether and filtered again through Celite. Filtrate was concentrated to give a racemic mixture of hexahydro-2H,2′H-2,4′-bipyran-4(3H)-one as light brown crystalline solid (1.78 g, 9.18 mmol, 83.0% yield). ¹H NMR (CDCl₃) δ: 4.30 (dd, J=11.0, 7.8 Hz, 1H), 4.01 (d, J=11.0 Hz, 2H), 3.63 (t, J=11.0 Hz, 1H), 3.27-3.47 (m, 3H), 2.60 (td, J=13.3, 7.5 Hz, 1H), 2.23-2.48 (m, 3H), 1.66-1.88 (m, 2H), 1.33-1.56 (m, 3H).

Methyl 2-bromobenzoate (0.3 g, 1.4 mmol, Eq: 1.00), hexahydro-2H,2′H-2,4′-bipyran-4(3H)-one (308 mg, 1.67 mmol, Eq: 1.2), Pd₂(dba)₃ (25.5 mg, 27.9 μmol, Eq: 0.02), xantphos (32.3 mg, 55.8 μmol, Eq: 0.04) and Cs₂CO₃ (591 mg, 1.81 mmol, Eq: 1.3) were placed in a microwave vial. Under N2, toluene (6 mL) was added. The resulting mixture stirred in the microwave (Biotage Initiator) at 135° C. for 70 minutes. The solvent was pipetted off onto a column for purification by flash chromatography (40 g SiO₂, hexanes/EtOAc 0-40% EtOAc). Product fractions were combined, concentrated to dryness and triturated it ether/hexanes—1/1. Solid was filtered off and dried to give a racemic mixture of 3-(tetrahydro-2H-pyran-4-yl)-3,4-dihydropyrano[4,3-c]isochromen-6(1H)-one as white solid (120 mg, 70% pure, 21% yield).

3-(Tetrahydro-2H-pyran-4-yl)-3,4-dihydropyrano[4,3-c]isochromen-6(1H)-one (0.117 g, 286 μmol, Eq: 1.00) stirred in ammonia in MeOH ˜7M (3.15 g, 4 ml, 28.0 mmol, Eq: 97.9) in the microwave (Biotage Initiator) at 140° C. for 1 h. Precipitate was filtered off, washed with little MeOH and dried to give a racemic mixture of 2-(tetrahydro-pyran-4-yl)-1,2,4,10-tetrahydro-3-oxa-10-aza-phenanthren-9-one as white crystalline solid (0.08 g, 278 μmol, 97% yield). ¹H NMR (CDCl₃) δ: 8.47 (d, J=7.8 Hz, 1H), 7.71-7.78 (m, 1H), 7.55 (t, J=7.5 Hz, 1H), 7.38 (d, J=8.0 Hz, 1H), 5.04 (d, J=14.1 Hz, 1H), 4.79 (d, J=14.3 Hz, 1H), 4.09 (dd, J=11.3, 3.5 Hz, 2H), 3.41-3.58 (m, 3H), 2.70-2.81 (m, 1H), 2.57 (d, J=16.1 Hz, 1H), 2.00 (d, J=13.6 Hz, 1H), 1.85 (ddd, J=11.5, 7.5, 3.8 Hz, 1H), 1.62-1.72 (m, 1H), 1.53 (quind, J=12.2, 4.5 Hz, 2H). MS calcd. for C₁₇H₁₉NO₃ [(M+H)⁺] 286.4, obsd. 286.

### Example 48

### rac-2-tert-Butyl-1,2,4,10-tetrahydro-3-oxa-5,10-diaza-phenanthren-9-one (I-48)

Methyl 2-bromonicotinate (0.47 g, 2.18 mmol, Eq: 1.00), 2-tert-butyldihydro-2H-pyran-4(3H)-one (408 mg, 2.61 mmol, Eq: 1.2), Pd₂(dba)₃ (39.8 mg, 43.5 μmol, Eq: 0.02), xantphos (50.4 mg, 87.0 μmol, Eq: 0.04) and Cs₂CO₃ (922 mg, 2.83 mmol, Eq: 1.3) were placed in a microwave vial. Under N₂, 1,2-dimethoxyethane (10 mL) was added. The resulting mixture stirred in the microwave (Biotage Initiator) at 135° C. for 80 minutes. The solvent was pipetted off onto a column for purification by flash chromatography (40 g SiO2, hexanes/EtOAc 0-25% EtOAc) to give 2-tert-butyl-1,4-dihydro-2H-3,10-dioxa-5-aza-phenanthren-9-one as a yellow oil (100 mg, 30% pure, 5% yield).

2-tert-Butyl-1,4-dihydro-2H-3,10-dioxa-5-aza-phenanthren-9-one (0.1 g, 30%, 116 μmol, Eq: 1.00) stirred in ammonia in MeOH ˜7M (1.57 g, 2 ml, 14.0 mmol, Eq: 121 in the microwave (Biotage Initiator) at 130° C. for 50 minutes. The precipitate was filtered off, washed with little MeOH and dried to give 2-tert-butyl-1,2,4,10-tetrahydro-3-oxa-5,10-diaza-phenanthren-9-one as off-white crystalline solid (0.015 g, 57.5 μmol, 49.7% yield). ¹H NMR (CDCl₃) δ: 8.92 (dd, J=4.8, 1.8 Hz, 1H), 8.68 (dd, J=8.0, 1.5 Hz, 1H), 7.42 (dd, J=8.0, 4.5 Hz, 1H), 5.25-5.35 (m, 1H), 4.78 (dt, J=15.0, 2.4 Hz, 1H), 3.44 (dd, J=11.0, 3.0 Hz, 1H), 2.73-2.85 (m, 1H), 2.43-2.52 (m, 1H), 1.07 (s, 9H). MS calcd. for C₁₅H₁₈N₂O₂ [(M+H)⁺] 259.3, obsd. 259.

### Example 49

### rac-2-tert-Butyl-6-methyl-1,2,4,10-tetrahydro-3-oxa-5,10-diaza-phenanthren-9-one (I-49)

Methyl 2-chloro-6-methylnicotinate (0.3 g, 1.62 mmol, Eq: 1.00), 2-tert-butyldihydro-2H-pyran-4(3H)-one (303 mg, 1.94 mmol, Eq: 1.2), prepared as described in above, Pd₂(dba)₃ (29.6 mg, 32.3 μmol, Eq: 0.02), xantphos (37.4 mg, 64.7 μmol, Eq: 0.04) and Cs₂CO₃ (685 mg, 2.1 mmol, Eq: 1.3) were placed in a microwave vial. Under N₂, toluene (7 mL) was added. The resulting mixture stirred in the microwave (Biotage Initiator) at 135° C. for 70 minutes. The solvent was pipetted off onto a column for purification by flash chromatography (40 g SiO₂, hexanes/EtOAc 0-40% EtOAc) to give 2-tert-butyl-6-methyl-1,4,4a,10a-tetrahydro-2H-3,10-dioxa-5-aza-phenanthren-9-one as light yellow oil (50 mg, 80% pure, 9% yield).

2-tert-Butyl-6-methyl-1,4,4a,10a-tetrahydro-2H-3,10-dioxa-5-aza-phenanthren-9-one (0.045 g, 132 μmol, Eq: 1.00) stirred in ammonia in MeOH ˜7M (18.8 μL, 132 μmol, Eq: 1.00) in the microwave (Biotage Initiator) at 130° C. for 50 minutes. The precipitate was filtered off, washed with little MeOH and dried to 2-tert-butyl-6-methyl-1,2,4,10-tetrahydro-3-oxa-5,10-diaza-phenanthren-9-one as off-white crystalline solid (0.018 g, 65.4 μmol, 49.7% yield). ¹H NMR (CDCl₃) δ: 8.53 (d, J=8.3 Hz, 1H), 7.26 (d, J=8.0 Hz, 1H), 5.29 (d, J=15.1 Hz, 1H), 4.76 (d, J=15.1 Hz, 1H), 3.41 (dd, J=10.9, 3.1 Hz, 1H), 2.71 (s, 4H), 2.45 (d, J=16.6 Hz, 1H), 1.06 (s, 9H). MS calcd. for C₁₆H₂₀N₂O₂ [(M+H)⁺] 273.4, obsd. 273.2.

### Example 50

### rac-2-[1-(Tetrahydro-pyran-4-carbonyl)-piperidin-4-yl]-1,2,4,10-tetrahydro-3-oxa-10-aza-phenanthren-9-one (I-50)

In a 5 mL round-bottomed flask, 4-(6-oxo-3,4,5,6-tetrahydro-1H-pyrano[4,3-c]isoquinolin-3-yl)piperidinium (31.4 mg, 110 μmol, Eq: 1.00), tetrahydro-2H-pyran-4-carbonyl chloride (24.5 mg, 165 μmol, Eq: 1.50) and DIPEA (42.7 mg, 57.7 μL, 330 μmol, Eq: 3.00) were combined with DMF (1 mL) to give an off-white suspension that was stirred at RT overnight. The mixture was diluted with 50 mL EtOAc, washed with 0.5 N HCl, sat NaHCO₃, H₂O, and evaporated to a white solid. The crude material (adsorbed onto silica) was purified by flash chromatography (silica gel, 12 g, 0% to 100% 5% MeOH/EtOAc in hexanes). Fractions were pooled and evaporated to yield 18.1 mg (42%) of 2-[1-(tetrahydro-pyran-4-carbonyl)-piperidin-4-yl]-1,2,4,10-tetrahydro-3-oxa-10-aza-phenanthren-9-one as a white solid. 1H NMR (DMSO-d6) δ: 11.18 (br. s., 1H), 8.19 (dd, J=7.9, 1.1 Hz, 1H), 7.62 (td, J=8.0, 1.0 Hz, 1H), 7.42 (t, J=7.7 Hz, 1H), 7.38 (d, J=8.3 Hz, 1H), 4.85 (m, 2H), 4.59 (d, J=14.7 Hz, 1H), 3.98 (d, J=12.8 Hz, 2H), 3.80 (m, 5H), 2.69 (m, 2H), 1.90 (m, 1H), 1.76 (m, 6H), 1.57 (m, 2H), 1.48-1.32 (m, 2H). MS calcd. for C₂₃H₂₈N₂O₄ [(M+H)⁺] 397.5, obsd. 397.1.

### Example 51

### rac-7-Isopropyl-5,7,8,9-tetrahydro-6-oxa-2,9-diaza-phenanthren-10-one (I-51)

7-Isopropyl-5,7,8,9-tetrahydro-6-oxa-2,9-diaza-phenanthren-10-one was synthesized following the procedure in Example 7. From 4-(4-oxo-tetrahydro-pyran-2-yl)-piperidine-1-carboxylic acid tert-butyl ester and 2-bromo-nicotinic acid methyl ester the crude material was purified by preparative TLC (ethyl acetate) 7-isopropyl-5,7,8,9-tetrahydro-6-oxa-2,9-diaza-phenanthren-10-one was obtained as a white solid (61 mg, 5%). ¹H NMR (DMSO-d₆) δ: 11.53 (s, 1H), 8.86 (dd, J=4.7, 1.7 Hz, 1H), 8.46 (dd, J=7.9, 1.9 Hz, 1H), 7.45 (dd, J=7.9, 4.5 Hz, 1H), 5.01 (d, J=15.1 Hz, 1H), 4.60 (d, J=14.7 Hz, 1H), 3.99 (d, J=14.7 Hz, 2H), 3.40-3.56 (m, 1H), 2.61-2.79 (m, 2H), 1.89 (d, J=12.1 Hz, 1H), 1.47-1.74 (m, 2H), 1.32-1.43 (m, 9H), 1.06-1.29 (m, 2H). MS calcd. for C₁₇H₁₉N₃O₄ [(M−tBu+H)⁺] 330.4, obsd. 330.

### Example 52

### rac-2-[1-(Oxetane-3-carbonyl)-piperidin-4-yl]-1,2,4,10-tetrahydro-3-oxa-10-aza-phenanthren-9-one (I-52)

In a 5 mL round-bottomed flask, 4-(6-oxo-3,4,5,6-tetrahydro-1H-pyrano[4,3-c]isoquinolin-3-yl)piperidinium (31.4 mg, 110 μmol, Eq: 1.00), DIPEA (42.7 mg, 57.7 μL, 330 μmol, Eq: 3.00) and oxetane-3-carboxylic acid (16.5 mg, 165 μmol, Eq: 1.50) were combined with DMF (1 mL) to give an off-white suspension. 1-(3-Dimethylaminopropyl-3-ethylcarbodiimide hydrochloride (31.6 mg, 165 μmol, Eq: 1.50) was added. The mixture was stirred at RT overnight. The reaction mixture was distributed into 50 mL EtOAc, washed with 10 mL 0.2 N HCl, water and sat NaHCO₃. The aqueous layers were back-extracted with 2×20 mL EtOAc. The combined EtOAc layers were dried over MgSO₄, filtered and evaporated to a white solid. The crude material (adsorbed onto silica) was purified by flash chromatography (silica gel, 15×12 g, 0% to 100% 5% MeOH/EtOAc in hexanes). Fractions were pooled and evaporated to 18.6 mg (46%) of 2-[1-(oxetane-3-carbonyl)-piperidin-4-yl]-1,2,4,10-tetrahydro-3-oxa-10-aza-phenanthren-9-one as a white solid. 1H NMR (DMSO-d6) δ: 11.20 (br. s., 1H), 8.19 (dd, J=7.9, 1.1 Hz, 1H), 7.64 (td, J=8.0, 1.0 Hz, 1H), 7.42 (t, J=7.7 Hz, 1H), 7.38 (d, J=8.3 Hz, 1H), 5.20 (m, 2H), 4.85 (m, 1H), 4.45 (m, 3H), 3.80 (m, 4H), 3.46 (m, 1H), 1.90 (m, 1H), 1.76 (m, 4H), 1.57 (m, 1H), 1.48-1.32 (m, 2H). MS calcd. for C₂₁H₂₄N₂O₄ [(M+H)⁺] 369.4, obsd. 369.0.

### Example 53

### rac-2-tert-Butyl-6-(1-hydroxy-1-methyl-ethyl)-1,2,4,10-tetrahydro-3-oxa-10-aza-phenanthren-9-one (I-53)

From 2-tert-butyl-tetrahydro-pyran-4-one and 2-bromo-terephthalic acid dimethyl ester: 2-tert-butyl-9-oxo-1,4,9,10-tetrahydro-2H-3-oxa-10-aza-phenanthrene-6-carboxylic acid methyl ester was obtained as a white solid (157 mg, 9%). ¹H NMR (DMSO-d₆) δ: 11.43 (br. s., 1H), 8.30 (d, J=7.9 Hz, 1H), 7.91 (s, 1H), 7.94 (dd, J=8.3, 1.5 Hz, 1H), 4.99 (d, J=14.0 Hz, 1H), 4.65 (d, J=14.0 Hz, 1H), 3.35 (dd, J=10.6, 3.8 Hz, 1H), 0.94 (s, 9H). MS calcd. for C₁₈H₂₁NO₄ [(M+H)⁺] 316.4, obsd. 316.

A solution of 2-tert-butyl-9-oxo-1,4,9,10-tetrahydro-2H-3-oxa-10-aza-phenanthrene-6-carboxylic acid methyl ester (75 mg, 0.238 mmol) in tetrahydrofuran (2 mL) was treated with dropwise addition of methylmagnesium bromide (0.32 mL, 3M in diethylether, 0.960 mmol). The mixture was stirred at room temperature for 18 h. The reaction mixture was quenched with an aqueous ammonium chloride solution, acidified with 1M HCl to pH 4, and extracted with ether. The organics were combined and concentrated in vacuo. The crude material was purified by HPLC (0% to 100% acetonitrile in water) to afford 2-tert-butyl-6-(1-hydroxy-1-methyl-ethyl)-1,2,4,10-tetrahydro-3-oxa-10-aza-phenanthren-9-one as a white solid (25 mg, 33%). ¹H NMR (DMSO-d₆) δ: 11.10 (s, 1H), 8.10 (d, J=8.7 Hz, 1H), 7.54 (dd, J=8.7, 1.5 Hz, 1H), 7.42 (d, J=1.1 Hz, 1H), 4.94 (d, J=14.0 Hz, 1H), 4.62 (d, J=14.4 Hz, 1H), 1.45 (s, 6H), 0.94 (s, 9H). MS calcd. for C₁₉H₂₅NO₃ [(M+H)⁺] 316.4, obsd. 316.

### Example 54

### 4-((S)-9-Oxo-1,4,9,10-tetrahydro-2H-3-oxa-10-aza-phenanthren-2-yl)-piperidine-1-carboxylic acid tert-butyl ester (I-54)

The racemic 4-(9-oxo-1,4,9,10-tetrahydro-2H-3-oxa-10-aza-phenanthren-2-yl)-piperidine-1-carboxylic acid tert-butyl ester was chromatographed using SFC separation OD column to give two optically pure isomers. The first peak was pooled and evaporated to yield 6 mg of 4-((S)-9-oxo-1,4,9,10-tetrahydro-2H-3-oxa-10-aza-phenanthren-2-yl)-piperidine-1-carboxylic acid tert-butyl ester as a white solid. MS calcd. for C₁₈H₂₀N₂O₄ [(M−tBu+H)⁺] 329.4, obsd. 329.1.

### Example 55

### 4-((R)-9-Oxo-1,4,9,10-tetrahydro-2H-3-oxa-10-aza-phenanthren-2-yl)-piperidine-1-carboxylic acid tert-butyl ester (I-55)

From the SFC separation above, the second peak was pooled and evaporated to yield 6 mg of 4-((R)-9-oxo-1,4,9,10-tetrahydro-2H-3-oxa-10-aza-phenanthren-2-yl)-piperidine-1-carboxylic acid tert-butyl ester as a white solid. MS calcd. for C₁₈H₂₀N₂O₄ [(M−tBu+H)⁺] 329.4, obsd. 329.1.

### Example 56

### rac-2-tert-Butyl-9-oxo-1,4,9,10-tetrahydro-2H-3-oxa-10-aza-phenanthrene-6-carbonitrile (I-56)

A solution of 2-tert-butyl-9-oxo-1,4,9,10-tetrahydro-2H-3-oxa-10-aza-phenanthrene-6-carboxylic acid amide (31 mg, 0.11 mmol) and triethylamine (0.50 ml, 3.59 mmol) in N-methylpyrrolidinone (0.4 mL) was placed under nitrogen and cooled to 0° C. Trifluoroacetic anhydride (0.170 ml, 1.21 mmol) was added dropwise and the mixture was allowed to warm to room temperature. The mixture was stirred for an additional 3 h at room temperature. The reaction was quenched with ice cold water. The precipitate was filtered and washed sequentially with water, 1N HCl, and hexane. The crude solid was agitated in a mixture of methanol/methylene chloride/hexane 1:9:5 and then filtered. The solids were collected and dried in vacuo to afford 2-tert-butyl-9-oxo-1,4,9,10-tetrahydro-2H-3-oxa-10-aza-phenanthrene-6-carbonitrile as an off-white solid (18 mg, 62%). ¹H NMR (DMSO-d₆) δ: 11.55 (s, 1H), 8.30 (d, J=8.3 Hz, 1H), 7.96 (d, J=1.1 Hz, 1H), 7.80 (dd, J=8.1, 1.3 Hz, 1H), 4.97 (d, J=14.4 Hz, 1H), 4.57 (d, J=14.4 Hz, 1H), 0.94 (s, 9H). MS calcd. for C₁₇H₁₈N₂O₂ [(M+H)⁺] 283.4, obsd. 283.

### Example 57

### rac-2-(2-Hydroxy-1,1-dimethyl-ethyl)-5-methyl-1,2,4,10-tetrahydro-3-oxa-10-aza-phenanthren-9-one (I-1)

2-(2-Benzyloxy-1,1-dimethyl-ethyl)-5-methyl-1,2,4,10-tetrahydro-3-oxa-10-aza-phenanthren-9-one was synthesized following the procedure in Example 7. From 2-(2-benzyloxy-1,1-dimethyl-ethyl)-tetrahydro-pyran-4-one and 2-bromo-3-methyl-benzoic acid methyl ester: the crude material was purified by column chromatography (SiO₂, 5% to 60% ethyl acetate in heptanes) to afford 2-(2-benzyloxy-1,1-dimethyl-ethyl)-5-methyl-1,2,4,10-tetrahydro-3-oxa-10-aza-phenanthren-9-one was obtained as a white solid (77 mg, 5%). ¹H NMR (DMSO-d₆) δ: 11.14 (s, 1H), 8.10 (d, J=7.9 Hz, 1H), 7.45 (d, J=6.8 Hz, 1H), 7.24-7.38 (m, 6H), 5.12 (d, J=14.0 Hz, 1H), 4.83 (d, J=14.7 Hz, 1H), 4.51 (d, J=12.5 Hz, 1H), 4.46 (d, J=12.5 Hz, 1H), 3.55 (dd, J=12.5, 1.9 Hz, 1H), 3.23-3.28 (m, 1H), 2.57 (s, 3H), 2.53-2.78 (m, 2H), 0.91 (s, 3H), 0.95 (s, 3H). MS calcd. for C₂₄H₂₇NO₃ [(M+H)⁺] 378.5, obsd. 378.

A mixture of 2-(2-benzyloxy-1,1-dimethyl-ethyl)-5-methyl-1,2,4,10-tetrahydro-3-oxa-10-aza-phenanthren-9-one (35 mg, 0.092 mmol), Pearlman's catalyst (50 mg), and HCl (1 drop) in dioxane (5 mL) was placed under a hydrogen atmosphere (3 bars) and reacted at room temperature for 28 h. The mixture was filtered through a pad of celite and the filtrate concentrated in vacuo to afford 2-(2-hydroxy-1,1-dimethyl-ethyl)-5-methyl-1,2,4,10-tetrahydro-3-oxa-10-aza-phenanthren-9-one as an off-white solid (24 mg, 92%). ¹H NMR (DMSO-d₆) δ: 11.15 (s, 1H), 8.10 (d, J=7.6 Hz, 1H), 7.45 (d, J=7.2 Hz, 1H), 7.28 (t, J=7.6 Hz, 1H), 5.13 (d, J=13.6 Hz, 1H), 4.88 (d, J=14.0 Hz, 1H), 4.57 (t, J=5.3 Hz, 1H), 3.51 (dd, J=11.3, 3.0 Hz, 1H), 3.16-3.30 (m, 2H), 2.57 (s, 3H), 2.36-2.45 (m, 1H), 0.89 (s, 3H), 0.82 (s, 3H). MS calcd. for C₁₇H₂₁NO₃ [(M+H)⁺] 288.4, obsd. 288.

### Example 58

### rac-2-[1-(1-methyl-cyclopropanecarbonyl)-piperidin-4-yl]-1,2,4,10-tetrahydro-3-oxa-5,10-diaza-phenanthren-9-one (I-57)

From 4-(5-methyl-9-oxo-1,4,9,10-tetrahydro-2H-3-oxa-10-aza-phenanthren-2-yl)-piperidine-1-carboxylic acid tert-butyl ester: 2-piperidin-4-yl-1,2,4,10-tetrahydro-3-oxa-5,10-diaza-phenanthren-9-one dihydrochloride was obtained as a yellow solid (35 mg, 91%): MS calcd. for C₁₆H₁₉N₃O₂ [(M+H)⁺] 286.4, obsd. 286.

From 2-piperidin-4-yl-1,2,4,10-tetrahydro-3-oxa-5,10-diaza-phenanthren-9-one dihydrochloride and 1-methyl-cyclopropanecarboxylic acid: 2-[1-(1-methyl-cyclopropanecarbonyl)-piperidin-4-yl]-1,2,4,10-tetrahydro-3-oxa-5,10-diaza-phenanthren-9-one was obtained as an off-white solid (13 mg, 37%). ¹H NMR (DMSO-d₆) δ: 11.53 (s, 1H), 8.86 (dd, J=4.5, 1.9 Hz, 1H), 8.46 (dd, J=8.1, 1.7 Hz, 1H), 7.45 (dd, J=7.9, 4.5 Hz, 1H), 5.02 (d, J=14.7 Hz, 1H), 4.60 (d, J=15.1 Hz, 1H), 4.31 (d, J=12.8 Hz, 2H), 3.46-3.55 (m, 1H), 2.67-2.94 (m, 2H), 1.96 (d, J=12.8 Hz, 1H), 1.68-1.85 (m, 1H), 1.63 (d, J=13.2 Hz, 1H), 1.00-1.36 (m, 2H), 1.21 (s, 3H), 0.70-0.80 (m, 2H), 0.43-0.64 (m, 2H). MS calcd. for C₂₁H₂₅N₃O₃ [(M+H)⁺] 368.5, obsd. 368.

### Example 59

### rac-7-tert-Butyl-4-methyl-5,7,8,9-tetrahydro-6-oxa-2,9-diaza-phenanthren-10-one (I-58)

A solution of lithium diisopropylamine (4.26 mL, 8.51 mmol) in tetrahydrofuran (12 mL) was cooled to −78° C. under argon. Chlorotrimethylsilane (1.1 mL, 8.51 mmol) was added to the cooled solution followed by dropwise addition of 2-tert-butyl-tetrahydro-pyran-4-one in tetrahydrofuran (30 mL). The resulting mixture was stirred at that temperature for an additional 2 h. The reaction was quenched by slow addition of saturated sodium bicarbonate (10 mL) and allowed to warm to room temperature. The resulting mixture was extracted with ether (2×50 mL). The combined organics was dried on sodium sulfate and concentrated in vacuo. The resulting residue was dissolved in dichloromethane (15 mL). This solution was slowly added to a solution of 5-methyl-nicotinic acid methyl ester (712 mg, 4.71 mmol), ethyl chloroformate (0.58 mL, 6.04 mmol) in dichloromethane (15 mL) which had been previously cooled to −10° C. and stirred at that temperature for 1 h. The resulting mixture was stirred for 4 days at room temperature under argon. The mixture was washed with 1M HCl, dried on sodium sulfate, and concentrated in vacuo. The residue was triturated with heptane and the triturate was concentrated in vacuo to give a brown oil (contains 20% desired material). The brown oil was dissolved in toluene (1 mL) and 2,3-dichloro-5,6-dicyano-1,4-benzoquinone (103 mg, 0.455 mmol) was added in one portion. The mixture was stirred at 100° C. for 1 h. After cooling to room temperature the mixture was diluted with ethyl acetate (25 mL), washed with saturated sodium bicarbonate, dried on sodium sulfate, and concentrated in vacuo. The residue was pre-absorbed onto silica and purified by column chomatography (SiO₂, 0% to 60% ethyl acetate in heptane) to afford 7-tert-butyl-4-methyl-7,8-dihydro-5H-6,9-dioxa-2-aza-phenanthren-10-one as a crude off-white solid (43 mg, 43%) that was taken on to the next step without further purification. MS calcd. for C₁₆H₁₉NO₃ [(M+H)⁺] 274.3, obsd. 274.

A mixture of 7-tert-butyl-4-methyl-7,8-dihydro-5H-6,9-dioxa-2-aza-phenanthren-10-one (60 mg, 0.220 mmol) in ammonia in methanol (7M, 10 mL) was heated at 140° C. in a microwave reactor for 1 h. The mixture was concentrate in vacuo and the resulting crude solid was taken up in a minimum amount of dichloromethane/heptane solution (2:8) and filtered. The desired product was allowed to precipitate from the filtrate. The precipitate was collected and dried in vacuo to afford 7-tert-butyl-4-methyl-5,7,8,9-tetrahydro-6-oxa-2,9-diaza-phenanthren-10-one as an off-white solid (25 mg, 43%). ¹H NMR (DMSO-d₆) δ: 11.52 (br. s., 1H), 9.15 (s, 1H), 8.45 (s, 1H), 5.20 (d, J=14.0 Hz, 1H), 4.86 (d, J=14.0 Hz, 1H), 2.52 (s, 3H), 0.93 (s, 9H). MS calcd, for C₁₆H₂₀N₂O₂ [(M+H)⁺] 273.4, obsd. 273.

### Example 60

### 2-(Tetrahydro-furan-3-yl)-1,2,4,10-tetrahydro-3-oxa-10-aza-phenanthren-9-one (I-59)

But-3-en-1-ol (1.09 g, 1.1 ml, 15.1 mmol, Eq: 1.00) and tetrahydrofuran-3-carbaldehyde (3.03 g, 30.3 mmol, Eq: 2) stirred in dichloromethane (50 mL) at 0° C. TFA (14.8 g, 10 ml, 130 mmol, Eq: 8.58) was added slowly. After the addition was complete the ice bath was removed and the clear light brown solution stirred at RT overnight. The solvent was mostly removed under reduced pressure. The residue was taken up in toluene and concentrated again. The remaining oil was taken up in methanol (30 mL) and was poured onto K₂CO₃ (5 g, 36.2 mmol, Eq: 2.39) and stirred for ˜3 h. The mixture was filtered through Celite and the filtrate was concentrated to dryness. The residue was partitioned between water and DCM. The aqueous layer was washed with DCM, the organic layers were combined, dried over Na₂SO₄, filtered and concentrated to give 2-(tetrahydrofuran-3-yl)tetrahydro-2H-pyran-4-ol as light brown oil (2.16 g, 83% yield). 2-(Tetrahydrofuran-3-yl)tetrahydro-2H-pyran-4-ol (2.16 g, 12.5 mmol, Eq: 1.00), silica gel (15 g, 12.5 mmol, Eq: 1.00) and PCC (3.51 g, 16.3 mmol, Eq: 1.3) stirred in dichloromethane (60 mL) at RT overnight. The mixture was filtered through Celite and the filtrate was concentrated. The remaining brown oil was purified by SiO₂ flash chromatography (40 g SiO₂, hexanes/EtOAc 0-45% EtOAc) to give 2-(tetrahydrofuran-3-yl)dihydro-2H-pyran-4(3H)-one as colorless oil as mixture of diastereomers (1 g, 48% yield).

Methyl 2-bromobenzoate (0.3 g, 1.4 mmol, Eq: 1.00), 2-(tetrahydrofuran-3-yl)dihydro-2H-pyran-4(3H)-one (285 mg, 1.67 mmol, Eq: 1.2), Pd₂(dba)₃ (38.3 mg, 41.9 μmol, Eq: 0.03), xantphos (48.4 mg, 83.7 μmol, Eq: 0.06) and Cs₂CO₃ (591 mg, 1.81 mmol, Eq: 1.3) were placed in a microwave vial. Under N₂, toluene (6 mL) was added. The resulting mixture stirred in the microwave (Biotage Initiator) at 130° C. for 70 minutes. The solvent was pipetted off onto a column for purification by flash chromatography (40 g SiO2, hexanes/EtOAc 0-40% EtOAc). The product fractions were combined, concentrated to dryness and triturated in hexanes/EtOAc ˜3/1. The solid was filtered off and dried to give 3-(tetrahydrofuran-3-yl)-3,4-dihydropyrano[4,3-c]isochromen-6(1H)-one as off-white sold as a mixture of diasteriomers (rac, 85/15, 37 mg, 9% yield). ¹H NMR (CDCl₃) δ: 8.36 (d, J=7.3 Hz, 1H), 7.72-7.81 (m, 1H), 7.54 (t, J=7.4 Hz, 1H), 7.23 (d, J=7.8 Hz, 1H), 4.88 (d, J=14.3 Hz, 1H), 4.69 (dt, J=14.2, 2.7 Hz, 1H), 3.90-4.05 (m, 2H), 3.76-3.89 (m, 2H), 3.61-3.70 (m, 1H), 2.64 (d, J=6.3 Hz, 2H), 2.44-2.58 (m, 1H), 2.10 (dtd, J=12.1, 7.9, 4.4 Hz, 1H), 1.69 (dq, J=12.3, 7.8 Hz, 1H).

A mixture of diastereomers of 3-(tetrahydrofuran-3-yl)-3,4-dihydropyrano[4,3-c]isochromen-6(1H)-one (0.035 g, 122 Eq: 1.00) stirred in ammonia in MeOH 7M (2.36 g, 3 ml, 139 mmol, Eq: 1140) in the microwave (Biotage Initiator) at 130° C. for 50 minutes. Precipitate was filtered off, washed with little MeOH and dried to give a mixture of diastereomers of 2-(tetrahydro-furan-3-yl)-1,2,4,10-tetrahydro-3-oxa-10-aza-phenanthren-9-one as pale brown crystalline solid (0.023 g, 83.9 μmol, 68.7% yield). ¹H NMR (CDCl₃) δ: 8.48 (d, J=7.5 Hz, 1H), 7.68-7.78 (m, 1H), 7.53 (t, J=7.5 Hz, 1H), 7.36 (d, J=8.0 Hz, 1H), 5.01 (d, J=13.3 Hz, 1H), 4.75-4.85 (m, 1H), 3.93-4.08 (m, 2H), 3.77-3.92 (m, 2H), 3.62-3.75 (m, 1H), 2.61-2.84 (m, 2H), 2.47-2.60 (m, 1H), 2.09-2.20 (m, 1H), 1.92-2.08 (m, 1H), 1.74 (dq, J=12.3, 7.8 Hz, 1H). MS calcd. for C₁₆H₁₇NO₃ [(M+H)⁺] 272.3, obsd. 272.

### Example 61

### rac-5-Methyl-2-(tetrahydro-pyran-4-yl)-1,2,4,10-tetrahydro-3-oxa-10-aza-phenanthren-9-one (I-60)

Methyl 2-bromo-3-methylbenzoate (0.3 g, 1.31 mmol, Eq: 1.00), hexahydro-2H,2′H-2,4′-bipyran-4(3H)-one (290 mg, 1.57 mmol, Eq: 1.2), Pd₂(dba)₃ (36.0 mg, 39.3 μmol, Eq: 0.03), xantphos (45.5 mg, 78.6 μmol, Eq: 0.06) and Cs₂CO₃ (555 mg, 1.7 mmol, Eq: 1.3) were placed in a microwave vial. Under N₂, toluene (6 mL) was added. The resulting mixture stirred in the microwave (Biotage Initiator) at 130° C. for 70 minutes. The solvent was pipetted off onto a column for purification by flash chromatography (40 g SiO₂, hexanes/EtOAc 0-40% EtOAc). Combined fractions were concentrated. The remaining oil was taken up in hexanes and little EtOAc was added. Precipitate was filtered off and dried to give a racemic mixture of 10-methyl-3-(tetrahydro-2H-pyran-4-yl)-3,4-dihydropyrano[4,3-c]isochromen-6(1H)-one as off-white solid (19 mg, 5% yield). ¹H NMR (CDCl₃) δ: 8.27 (d, J=7.5 Hz, 1H), 7.52 (d, J=7.0 Hz, 1H), 7.36-7.43 (m, 1H), 5.11 (d, J=13.8 Hz, 1H), 4.91 (dt, J=14.1, 2.9 Hz, 1H), 4.07 (dd, J=11.2, 3.9 Hz, 2H), 3.41-3.51 (m, 3H), 2.53-2.72 (m, 5H), 1.97 (d, J=14.8 Hz, 1H), 1.80 (dtd, J=11.6, 7.7, 4.0 Hz, 1H), 1.41-1.66 (m, 6H).

10-Methyl-3-(tetrahydro-2H-pyran-4-yl)-3,4-dihydropyrano[4,3-c]isochromen-6(1H)-one (0.016 g, 53.3 μmol, Eq: 1.00) stirred in ammonia in MeOH 7M (1.57 g, 2 ml, 14.0 mmol, Eq: 263) in the microwave (Biotage Initiator) at 130° C. for 45 minutes. The precipitate was filtered off, washed with little MeOH and dried to give a racemic mixture of 5-methyl-2-(tetrahydro-pyran-4-yl)-1,2,4,10-tetrahydro-3-oxa-10-aza-phenanthren-9-one as white needles (0.009 g, 30.1 μmol, 56.4% yield). ¹H NMR (CDCl₃) δ: 8.40 (d, J=7.8 Hz, 1H), 7.50 (d, J=7.0 Hz, 1H), 7.35-7.43 (m, 1H), 5.24 (d, J=13.8 Hz, 1H), 5.03 (d, J=14.1 Hz, 1H), 4.09 (dd, J=11.3, 4.0 Hz, 2H), 3.41-3.54 (m, 3H), 2.72-2.85 (m, 1H), 2.57-2.71 (m, 4H), 1.99 (d, J=13.3 Hz, 1H), 1.82 (dtd, J=11.6, 7.7, 3.8 Hz, 1H), 1.66 (br. s., 1H), 1.44-1.61 (m, 2H). MS calcd. for C₁₈H₂₁NO₃ [(M+H)⁺] 300.4, obsd. 300.

### Example 62

### rac-5-Methyl-2-[1-(1-methyl-cyclopropanecarbonyl)-piperidin-4-yl]-1,2,4,10-tetrahydro-3-oxa-10-aza-phenanthren-9-one (I-61)

A mixture of 4-(9-oxo-1,4,9,10-tetrahydro-2H-3-oxa-10-aza-phenanthren-2-yl)-piperidine-1-carboxylic acid tert-butyl ester (0.249 g, 0.738 mmol) in anhydrous methanol (50 mL) was cooled to 0° C. Acetyl chloride (1 mL, 14.1 mmol) was added dropwise. After 5 min., the mixture was allowed to stir at room temperature for 24 h. The resulting mixture was concentrated in vacuo to afford 5-methyl-2-piperidin-4-yl-1,2,4,10-tetrahydro-3-oxa-10-aza-phenanthren-9-one hydrochloride as an off-white solid (252 mg, quant.) that was taken to subsequent reactions without further purification. MS calcd. for C₁₈H₂₂N₂O₂ [(M+H)⁺] 299.4, obsd. 299.

5-Methyl-2-[1-(1-methyl-cyclopropanecarbonyl)-piperidin-4-yl]-1,2,4,10-tetrahydro-3-oxa-10-aza-phenanthren-9-one was synthesized following the procedure in Example 26. From 5-methyl-2-piperidin-4-yl-1,2,4,10-tetrahydro-3-oxa-10-aza-phenanthren-9-one hydrochloride and 1-methyl-cyclopropanecarboxylic acid: the crude material was purified by column chromatography (SiO2, 5% to 100% ethyl acetate in heptane) to afford 5-methyl-2-[1-(1-methyl-cyclopropanecarbonyl)-piperidin-4-yl]-1,2,4,10-tetrahydro-3-oxa-10-aza-phenanthren-9-one as an off-white solid (26 mg, 29%). ¹H NMR (DMSO-d₆) δ: 11.19 (s, 1H), 8.11 (dd, J=7.9, 1.5 Hz, 1H), 7.45 (d, J=6.8 Hz, 1H), 7.29 (t, J=7.6 Hz, 1H), 5.12 (d, J=14.0 Hz, 1H), 4.89 (d, J=14.0 Hz, 1H), 4.30 (d, J=12.1 Hz, 2H), 3.41 (q, J=7.3 Hz, 1H), 2.78 (br. s., 2H), 2.57 (s, 3H), 1.95 (br. d, J=12.5 Hz, 1H), 1.62 (br. d, J=14.0 Hz, 1H), 1.53-1.78 (m, 1H), 1.07-1.28 (m, 2H), 1.21 (s, 3H), 0.67-0.88 (m, 2H), 0.52 (s, 2H). MS calcd. for C₂₃H₂₈N₂O₃ [(M+H)⁺] 381.5, obsd. 381.

### Example 63

### rac-4-(5-Methyl-9-oxo-1,4,9,10-tetrahydro-2H-3-oxa-10-aza-phenanthren-2-yl)-piperidine-1-carboxylic acid tert-butyl ester (I-62)

A mixture of methyl 2-bromo-3-methylbenzoate (1 g, 4.37 mmol), tert-butyl 4-(4-oxotetrahydro-2H-pyran-2-yl)piperidine-1-carboxylate (1.42 g, 5.02 mmol), Cs₂CO₃ (1.85 g, 5.68 mmol), xantphos (101 mg, 175 μmol) and Pd₂(dba)₃ (80.0 mg, 87.3 μmol) in dioxane (10 mL) was placed under argon and heated at 135° C. in a microwave reactor for 80 min. The reaction mixture was pre-absorbed onto silica and purified by flash chromatography (SiO₂, 0% to 40% EtOAc in heptane) to afford a viscous oil. The crude oil was triturated to give a white solid that was dissolved in a solution of ammonia in methanol (7M, 10 mL). The reaction mixture was heated at 140° C. in a microwave reactor for 1 h. The precipitate was filtered and dried in vacuo to afford 4-(5-methyl-9-oxo-1,4,9,10-tetrahydro-2H-3-oxa-10-aza-phenanthren-2-yl)-piperidine-1-carboxylic acid tert-butyl ester as an off-white solid (0.314 g, 18%). ¹H NMR (DMSO-d₆) δ: 11.19 (br. s., 1H), 8.11 (d, J=6.8 Hz, 1H), 7.45 (d, J=6.8 Hz, 1H), 7.29 (t, J=7.6 Hz, 1H), 5.11 (d, J=13.6 Hz, 1H), 4.89 (d, J=14.0 Hz, 1H), 3.98 (d, J=13.2 Hz, 2H), 3.34-3.46 (m, 2H), 2.62-2.76 (m, 2H), 2.57 (s, 3H), 1.89 (d, J=12.8 Hz, 1H), 1.45-1.74 (m, 2H), 1.39 (s, 9H), 1.33-1.42 (m, 1H), 0.93-1.19 (m, 2H). MS calcd. for C₁₉H₂₂N₂O₄ [(M−tBu+H)⁺] 343.4, obsd. 343.

### Example 64

### rac-2-(1-Cyclopropanecarbonyl-piperidin-4-yl)-5-methyl-1,2,4,10-tetrahydro-3-oxa-10-aza-phenanthren-9-one (I-63)

2-(1-Cyclopropanecarbonyl-piperidin-4-yl)-5-methyl-1,2,4,10-tetrahydro-3-oxa-10-aza-phenanthren-9-one was synthesized following the procedure in Example 24. From 5-methyl-2-piperidin-4-yl-1,2,4,10-tetrahydro-3-oxa-10-aza-phenanthren-9-one hydrochloride and cyclopropanecarbonyl chloride: the crude residue was precipitated with water to afford 2-(1-cyclopropanecarbonyl-piperidin-4-yl)-5-methyl-1,2,4,10-tetrahydro-3-oxa-10-aza-phenanthren-9-one as an off white solid (39 mg, 59%). ¹H NMR (DMSO-d₆) δ: 11.20 (s, 1H), 8.11 (dd, J=7.9, 1.1 Hz, 1H), 7.46 (d, J=7.6 Hz, 1H), 7.22-7.36 (m, 1H), 5.12 (d, J=14.0 Hz, 1H), 4.89 (d, J=14.4 Hz, 1H), 4.22-4.50 (m, 2H), 3.07 (br. s., 1H), 2.57 (s, 3H), 1.85-2.08 (m, 2H), 1.46-1.82 (m, 2H), 1.19 (br. s., 2H), 0.63-0.84 (m, 4H). MS calcd. for C₂₂H₂₆N₂O₃ [(M+H)⁺] 367.5, obsd. 367.

### Example 65

### rac-2-(1-Hydroxy-1-methyl-ethyl)-5-methyl-1,2,4,10-tetrahydro-3-oxa-10-aza-phenanthren-9-one (I-64)

2-(Benzyloxy)-2-methylpropan-1-ol (3 g, 16.6 mmol, Eq: 1.00), silica gel (15 g, 16.6 mmol, Eq: 1.00) and PCC (4.31 g, 20.0 mmol, Eq: 1.2) stirred in dichloromethane (60 mL) at RT overnight. The mixture was filtered through Celite. The filtrate was concentrated and purified by SiO₂ flash chromatography (80 g SiO₂, hexanes/EtOAc 0-20% EtOAc) to give 2-(benzyloxy)-2-methylpropanal as colorless oil (2 g, 90% pure, 60% yield).

But-3-en-1-ol (595 mg, 0.6 ml, 8.25 mmol, Eq: 1.00) and 2-(benzyloxy)-2-methylpropanal (2.02 g, 11.3 mmol, Eq: 1.37) stirred in dichloromethane (50 mL) at 0° C. TFA (8.88 g, 6 ml, 77.9 mmol, Eq: 9.43) was added slowly. After the addition was complete the reaction mixture was allowed to warm up to RT overnight. The solvent was mostly removed under reduced pressure. The residue was taken up in toluene and concentrated again. The remaining oil was taken up in methanol (50 mL) and was poured onto Na₂CO₃ (10 g, 94.3 mmol, Eq: 11.4) and stirred at RT for 3 h. The mixture was filtered through Celite and the filtrate was concentrated to dryness. The residue was partitioned between water and DCM. The aqueous layer was washed with DCM, the organic layers were extracted with brine, combined, dried over Na₂SO₄, filtered and concentrated to give crude 2-(2-(benzyloxy)propan-2-yl)tetrahydro-2H-pyran-4-ol as a light yellow oil (2.05 g, 99% yield) which was used without further purification.

2-(2-(Benzyloxy)propan-2-yl)tetrahydro-2H-pyran-4-ol (2.05 g, 8.19 mmol, Eq: 1.00), silica gel (10 g, 8.19 mmol, Eq: 1.00) and PCC (2.65 g, 12.3 mmol, Eq: 1.5) stirred in dichloromethane (50 mL) at RT overnight. The mixture was filtered through Celite, the filtrate was concentrated and purified by SiO₂ flash chromatography (40 g SiO₂, hexanes/EtOAc 0-30% EtOAc) to give 2-(2-(benzyloxy)propan-2-yl)dihydro-2H-pyran-4(3H)-one as a white crystalline solid (975 mg, 85% pure, 40% yield).

Methyl 2-bromo-3-methylbenzoate (0.3 g, 1.31 mmol, Eq: 1.00), 2-(2-(benzyloxy)propan-2-yl)dihydro-2H-pyran-4(3H)-one (421 mg, 1.44 mmol, Eq: 1.1), tris(dibenzylideneacetone)-dipalladium (0) (48.0 mg, 52.4 μmol, Eq: 0.04), xantphos (60.6 mg, 105 μmol, Eq: 0.08) and cesium carbonate (555 mg, 1.7 mmol, Eq: 1.3) were placed in a microwave vial under N₂. Dioxane (8 mL) was added and the mixture stirred in the microwave (Biotage Initiator) at 135° C. for 75 minutes. The solvent was partially removed and the residue was purified by SiO₂ flash chromatography (40 g SiO₂, hexanes/EtOAc 0-15% EtOAc). Product containing fractions were combined, concentrated and triturated in hexanes. Solid was filtered off and dried to give 3-(2-(benzyloxy)propan-2-yl)-10-methyl-3,4-dihydropyrano[4,3-c]isochromen-6(1H)-one as off-white solid (22 mg, 5% yield). ¹H NMR (CDCl₃) δ: 8.27 (d, J=7.3 Hz, 1H), 7.51 (d, J=7.3 Hz, 1H), 7.31-7.44 (m, 6H), 5.17 (d, J=14.1 Hz, 1H), 4.90 (dt, J=14.0, 2.9 Hz, 1H), 4.61 (q, J=11.5 Hz, 2H), 3.71 (dd, J=11.0, 3.5 Hz, 1H), 2.95 (dd, J=17.7, 11.7 Hz, 1H), 2.61-2.71 (m, 1H), 2.59 (s, 3H), 1.39 (d, J=11.3 Hz, 6H). MS calcd. for C₂₃H₂₄O₄ [(M+H)⁺] 365.5, obsd. 365.

3-(2-(Benzyloxy)propan-2-yl)-10-methyl-3,4-dihydropyrano[4,3-c]isochromen-6(1H)-one (0.021 g, 57.6 μmol, Eq: 1.00) stirred in ammonia in MeOH 7M (1.57 g, 2 ml, 14.0 mmol, Eq: 243) in the microwave (Biotage Initiator) at 130° C. for 45 minutes. The solvent was removed to give 3-(2-(benzyloxy)propan-2-yl)-10-methyl-3,4-dihydro-1H-pyrano[4,3-c]isoquinolin-6(5H)-one as off-white solid (20 mg, 95% yield). ¹H NMR (CDCl₃) δ: 8.38 (d, J=7.5 Hz, 1H), 7.47 (d, J=6.8 Hz, 1H), 7.32-7.41 (m, 6H), 5.29 (d, J=13.8 Hz, 1H), 5.01 (d, J=13.8 Hz, 1H), 4.58-4.69 (m, 2H), 3.72 (dd, J=11.0, 3.5 Hz, 1H), 2.92-3.06 (m, 1H), 2.58-2.69 (m, 4H), 1.42 (d, J=13.6 Hz, 6H). MS calcd. for C₂₃H₂₅NO₃ [(M+H)⁺] 364.5, obsd. 364.

3-(2-(Benzyloxy)propan-2-yl)-10-methyl-3,4-dihydro-1H-pyrano[4,3-c]isoquinolin-6(5H)-one (0.02 g, 55.0 μmol, Eq: 1.00) and Pd(OH)₂ 20 wt % (10 mg, 71.2 μmol, Eq: 1.29) were suspended in dioxane (0.7 mL). One drop of acetic acid was added and the mixture stirred at 1 atm of H₂ at RT for 3 h. The mixture was placed in a Parr vessel. More dioxane (2 mL) and a drop of conc HCl was added. The mixture was placed under 45 psi H₂ and shaken at RT for 5 h. Acetic acid (1 mL) and more Pd(OH)₂ 20 wt % (10 mg, 71.2 μmol, Eq: 1.29) was added and the mixture was returned to the Parr at 50 psi for 3 days. The reaction mixture was filtered through Celite. The filtrate was passed through the H-cube hydrogenator at 1 ml/min, 50° C., 10 bar. The solvent was removed under reduced pressure. The residue was purified by SiO₂ flash chromatography (4 g SiO₂, DCM/MeOH 0-4% MeOH) to give 2-(1-hydroxy-1-methyl-ethyl)-5-methyl-1,2,4,10-tetrahydro-3-oxa-10-aza-phenanthren-9-one as a white solid (5 mg, 32% yield). ¹H NMR (CDCl₃) δ: 8.39 (d, J=7.5 Hz, 1H), 7.53-7.58 (m, 1H), 7.41-7.47 (m, 1H), 5.36 (s, 1H), 5.11 (d, J=14.1 Hz, 1H), 3.59 (dd, J=11.0, 3.5 Hz, 1H), 2.94-3.04 (m, 1H), 2.70 (s, 3H), 2.56 (d, J=16.8 Hz, 1H), 1.37 (s, 3H), 1.30 (s, 3H). MS calcd. for C₁₆H₁₉NO₃ [(M+H)⁺] 274.3, obsd. 274.

### Example 66

### rac-2-(3-Methyl-oxetan-3-yl)-1,2,4,10-tetrahydro-3-oxa-10-aza-phenanthren-9-one (I-65)

To a sealed vial containing a solution of but-3-en-1-ol (500 mg, 6.93 mmol) and 3-methyloxetane-3-carbaldehyde (1.53 g, 15.3 mmol) in anhydrous DCM cooled in ice-water bath was added TFA (5 ml, 64.9 mmol) dropwise. The solution was then stirred at room temperature for 24 hours. The solvent was then removed under reduced pressure. The residue was co-evaporated with toluene twice, followed by treatment with MeOH and sodium carbonate. The resulting mixture was stirred overnight at room temperature, diluted with water, and extracted with EtOAc (100 mL×3). The combined EtOAc solution was washed with water and brine, filtered and evaporated to give 2-(3-methyloxetan-3-yl)tetrahydro-2H-pyran-4-ol as a light colored oil (345 mg, 28.9%). The product was used directly in the next step.

A mixture of 2-(3-methyloxetan-3-yl)tetrahydro-2H-pyran-4-ol (340 mg, 1.97 mmol), PCC (638 mg, 2.96 mmol), and silica gel (5 g) in methylene chloride (30 mL) was stirred at room temperature overnight. The mixture was filtered through celite. The filtrate was evaporated to give 2-(3-methyloxetan-3-yl)dihydro-2H-pyran-4(3H)-one as a colorless oil (138 mg, 41.1%). The product was used directly in the next step.

To a microwave reaction vessel was added methyl 2-bromobenzoate (140 mg, 0.65 mmol), 2-(3-methyloxetan-3-yl)dihydro-2H-pyran-4(3H)-one (133 mg, 0.78 mmol), cesium carbonate (276 mg, 0.85 mmol) and toluene (2.5 mL). The vessel was sealed and nitrogen gas was bubbled through the mixture for 10 minutes followed by opening the vessel and immediately adding xantphos (22.6 mg, 0.04 mmol) and Pd₂(dba)₃ (18 mg, 0.02 mmol). The vessel was quickly sealed and nitrogen gas was bubbled through the mixture for another 5 minutes. The mixture was then heated at 130° C. for 80 minutes in a Biotage microwave reactor, cooled to room temperature, diluted with EtOAc (4 mL), stirred for 20 minutes, and filtered. The filtrate was evaporated. The residue was purified by column (0-50% EtOAc in hexane) to give 3-(3-methyloxetan-3-yl)-3,4-dihydropyrano[4,3-c]isochromen-6(1H)-one (95 mg, 53.6%). MS calcd. for C₁₆H₁₆O₄ [(M+H)⁺] 273.3, obsd. 272.8.

To a microwave reactor vessel was charged with 3-(3-methyloxetan-3-yl)-3,4-dihydropyrano[4,3-c]isochromen-6(1H)-one (95 mg, 0.35 μmol) and ammonia in MeOH (7M, 4 mL, 28 mmol). The mixture was then heated to 130° C. for 2 hours in a Biotage microwave reactor. The light colored solution was evaporated and the solid crude product was purified by column (0-5% MeOH in DCM) to give title compound as an off-white solid (45 mg, 47.5%). ¹H NMR (300 MHz, DMSO-d₆) δ ppm 11.11 (s, 1H), 8.48 (d, 1H), 7.73 (t, 1H), 7.53 (t, 1H), 7.38 (d, 1H), 5.13 (d, 1H), 4.90-4.77 (m, 3H), 4.46 (dd, 2H), 3.92 (dd, 1H), 2.72 (t, 1H), 2.58 (d, 1H), 1.48 (s, 3H). MS calcd. for C₁₆H₁₇NO₃ [(M+H)⁺] 272.3, obsd. 271.8.

### Example 67

### (S)-2-(2-Hydroxy-1,1-dimethyl-ethyl)-5-methyl-1,2,4,10-tetrahydro-3-oxa-10-aza-phenanthren-9-one (I-66)

The racemic 2-(2-hydroxy-1,1-dimethyl-ethyl)-5-methyl-1,2,4,10-tetrahydro-3-oxa-10-aza-phenanthren-9-one (40 mg) was chromatographed using SFC separation OD column to give two optically pure isomers. The first peak was pooled and evaporated to yield 14.4 mg of (S)-2-(2-hydroxy-1,1-dimethyl-ethyl)-5-methyl-1,2,4,10-tetrahydro-3-oxa-10-aza-phenanthren-9-one as a white solid. ¹H NMR (300 MHz, DMSO-d₆) δ ppm 11.15 (s, 1H) 8.10 (d, J=7.55 Hz, 1H) 7.45 (d, J=7.18 Hz, 1H) 7.20-7.37 (m, 1H) 5.13 (d, J=13.98 Hz, 1H) 4.88 (d, J=13.60 Hz, 1H) 4.57 (t, J=5.29 Hz, 1H) 3.51 (dd, J=10.95, 3.02 Hz, 1H) 3.17-3.28 (m, 2H) 2.57 (s, 3H) 2.35-2.45 (m, 1H) 0.89 (s, 3H) 0.82 (s, 3H). MS calcd. for C₁₇H₁₂NO₃ [(M+H)⁺] 288.4, obsd. 288.2.

### Example 68

### (R)-2-(2-Hydroxy-1,1-dimethyl-ethyl)-5-methyl-1,2,4,10-tetrahydro-3-oxa-10-aza-phenanthren-9-one (I-67)

From the SFC separation above, the second peak was pooled and evaporated to yield 14.9 mg of (R)-2-(2-hydroxy-1,1-dimethyl-ethyl)-5-methyl-1,2,4,10-tetrahydro-3-oxa-10-aza-phenanthren-9-one as a white solid. ¹H NMR (300 MHz, DMSO-d₆) δ ppm 11.15 (br. s., 1H) 8.10 (d, J=7.93 Hz, 1H) 7.45 (d, J=7.18 Hz, 1H) 7.22-7.37 (m, 1H) 5.13 (d, J=13.98 Hz, 1H) 4.88 (d, J=13.60 Hz, 1H) 4.57 (t, J=5.29 Hz, 1H) 3.44-3.59 (m, 1H) 3.13-3.28 (m, 1H) 2.57 (s, 3H) 2.30-2.45 (m, 1H) 0.89 (s, 3H) 0.82 (s, 3H). MS calcd. for C₁₇H₁₂NO₃ [(M+H)⁺] 288.4, obsd. 288.3.

### Example 69

### 10-methyl-3-[1-(oxetan-3-yl)-4-piperidyl]-1 3 4 5-tetrahydropyrano[4,3-c]isoquinolin-6-one (I-71)

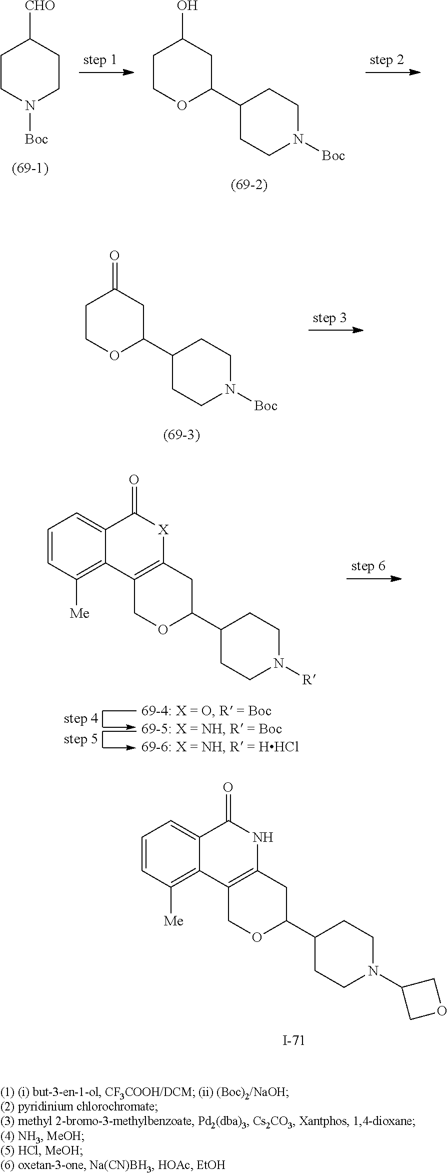

step 1: To a stirred solution of but-3-en-1-ol (2 g, 27.74 mmol, 1.00 equiv) and tert-butyl 4-formylpiperidine-1-carboxylate (12 g, 56.27 mmol, 2.03 equiv) in DCM (200 mL) maintained under nitrogen at 0° C. was added dropwise trifluoroacetic acid (100 mL). The resulting solution was stirred overnight at RT and then concentrated under vacuum. The residue was diluted with 200 mL of water then sodium hydroxide (10 g, 250.00 mmol, 9.01 equiv) was added in portions. The resulting solution was stirred for another 2 h at RT then a solution of (Boc)₂O (15 g, 68.73 mmol, 2.48 equiv) in THF (100 mL) was added. The reaction mixture was stirred overnight at RT and the excess THF was removed under vacuum. The resulting solution was extracted with 2×200 mL of EtOAc. The combined organic layers were dried (Na₂SO₄), filtered and concentrated under vacuum. The residue was purified by SiO₂ chromatography eluting with EtOAc/petroleum ether (4:1) to afford 4.5 g (57%) of tert-butyl 4-(4-hydroxyoxan-2-yl)piperidine-1-carboxylate as a white solid: MS calcd. for C15H28NO4 [(M+H)+]: 286.0, obsd. 286.0.

step 2: A mixture of tert-butyl 4-(4-hydroxyoxan-2-yl)piperidine-1-carboxylate (4.5 g, 15.77 mmol, 1.00 equiv), silica gel (15 g) and PCC (5 g, 23.20 mmol, 1.47 equiv) in DCM (100 mL) was stirred overnight at RT. The solid material was removed by filtration. The filtrate was washed with 2×100 mL of water then dried (Na₂SO₄), filtered and concentrated under vacuum. The residue was purified by SiO2 chromatography eluting with EtOAc/petroleum ether (1:5) to give 3.8 g (85%) of tert-butyl 4-(4-oxooxan-2-yl)piperidine-1-carboxylate as a white solid: TLC: R_{f}=0.4, ethyl acetate/petroleum ether=1:1.

step 3: A mixture of tert-butyl 4-(4-oxooxan-2-yl)piperidine-1-carboxylate (3.8 g, 13.41 mmol, 1.00 equiv), methyl 2-bromo-3-methylbenzoate (3 g, 13.10 mmol, 0.98 equiv), Pd₂(dba)₃.CHCl₃ (0.5 g), Cs₂CO₃ (4.8 g, 14.73 mmol, 1.10 equiv) and XantPhos (500 mg, 0.86 mmol, 0.06 equiv) in 1,4-dioxane (100 mL) was stirred under nitrogen at 110° C. overnight. The reaction mixture was cooled to RT, diluted with 300 mL of ethyl acetate and washed with 2×100 mL of water. The organic layer was dried (Na₂SO₄), filtered and concentrated under vacuum. The residue was purified by SiO₂ chromatography eluting with ethyl acetate/petroleum ether (1:3) to afford 2.5 g of crude tert-butyl 4-[10-methyl-6-oxo-1H,3H,4H,6H-pyrano[4,3-c]isochromen-3-yl]piperidine-1-carboxylate as a red oil. TLC: R_{f}=0.5, ethyl acetate/petroleum ether=1:1

step 4: A solution of tert-butyl 4-[10-methyl-6-oxo-1H,3H,4H,6H-pyrano[4,3-c]isochromen-3-yl]piperidine-1-carboxylate (1.5 g, 3.75 mmol, 1.00 equiv) in a saturated solution of ammonia in MeOH (20 mL) was stirred in a 30-mL sealed tube at 120° C. overnight. The reaction mixture was cooled to RT and concentrated under vacuum. The residue was purified by SiO₂ chromatography eluting with dichloromethane/MeOH (100:5) to give 450 mg (30%) of tert-butyl 4-[10-methyl-6-oxo-1H,3H,4H,5H,6H-pyrano[4,3-c]isoquinolin-3-yl]piperidine-1-carboxylate as a light yellow solid: MS calcd. for C₂₃H₃₁N₂O₄ [(M+H)+]: 399.0, obsd. 399.0.

step 5: A solution of tert-butyl 4-[10-methyl-6-oxo-1H,3H,4H,5H,6H-pyrano[4,3-c]isoquinolin-3-yl]piperidine-1-carboxylate (450 mg, 1.13 mmol, 1.00 equiv) in a saturated solution of hydrogen chloride in MeOH (30 mL) was stirred at RT for 3 h. The reaction mixture was concentrated under vacuum to give 340 mg (90%) of 10-methyl-3-(piperidin-4-yl)-1H, 3H,4H,5H,6H-pyrano[4,3-c]isoquinolin-6-one hydrochloride as a yellow solid: MS calcd. for C₁₈H₂₄ClN₂O₂ [(M+H-HCl)+]: 299.0, obsd. 299.0.

step 6: A solution of 10-methyl-3-(piperidin-4-yl)-1H,3H,4H,5H,6H-pyrano[4,3-c]isoquinolin-6-one hydrochloride (80 mg, 0.24 mmol, 1.00 equiv) and oxetan-3-one (20 mg, 0.28 mmol, 1.16 equiv) in EtOH (15 mL) was stirred at 60° C. for 1 h. Acetic acid (0.1 mL) followed by NaB(CN)H₃ (45 mg) were added and the resulting solution was stirred at 60° C. overnight. Additional oxetan-3-one (20 mg, 0.28 mmol, 1.16 equiv) and NaB(CN)H₃ (45 mg) were then added and the resulting solution was stirred for another 3 h at 60° C. The reaction mixture was cooled to RT then concentrated under vacuum. The mixture was diluted with 50 mL of water and then extracted with 2×60 mL of DCM. The organic combined layers was dried (Na₂SO₄), filtered and concentrated under vacuum. The crude product was purified by Prep-HPLC with the following conditions: Column, Sunfire C18 19×150, mobile phase, CH₃CN:NH₄CO₃/H₂O (10 mmol/L)=20%-65%, 20 min, Detector UV 254 nm to afford 5.5 mg (6%) of 10-methyl-3-[1-(oxetan-3-yl)piperidin-4-yl]-1H,3H,4H,5H,6H-pyrano[4,3-d]soquinolin-6-one as a white solid: ¹H-NMR (300 MHz, DMSO-d₆) δ ppm 11.19 (s, 1H), 8.13 (d, J=6.9 Hz, 1H), 7.47 (d, J=6.6 Hz, 1H), 7.33-7.27 (m, 1H), 5.14 (d, J=13.8 Hz, 1H), 4.93 (d, J=14.1 Hz, 1H), 4.54-4.50 (m, 2H), 4.43-4.39 (m, 2H), 3.43-3.31 (m, 2H), 2.75-2.71 (m, 2H), 2.59 (s, 3H), 2.51-2.49 (m, 2H), 1.96-1.85 (m, 1H), 1.77-1.68 (m, 2H), 1.61-1.51 (m, 1H), 1.45-1.20 (m, 3H); MS calcd. for C₂₁H₂₇N₂O₃ [(M+H)+]: 355.0, obsd. 355.1.

10-Methyl-3-(1-tetrahydropyran-4-yl-4-piperidyl)-1 3 4 5-tetrahydropyrano[4,3-c]isoquinolin-6-one (I-72) was prepared analogously except in step 6, oxetan-3-one was replaced with dihydro-2H-pyran-4(3H)-one. The parent ion of the product was m/e=383.15.

### Example 70

### 3-[1-(2-methoxyethyl)-4-piperidyl]-10-methyl-1 3 4 5-tetrahydropyrano[4,3-c]isoquinolin-6-one (I-73)

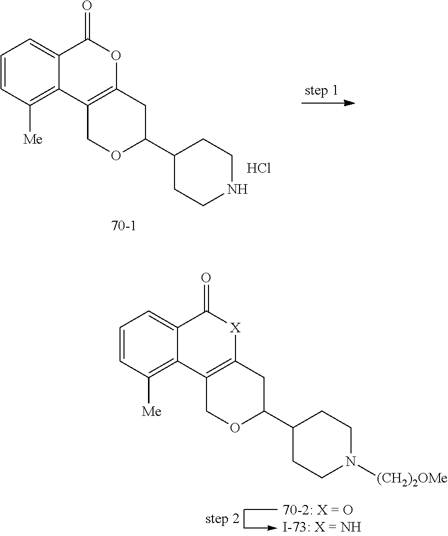

step 1: A mixture of 10-methyl-3-(piperidin-4-yl)-1H,3H,4H,6H-pyrano[4,3-c] isochromen-6-one hydrochloride (350 mg, 1.04 mmol, 1.00 equiv), 1-bromo-2-methoxyethane (250 mg, 1.80 mmol, 1.73 equiv) and potassium carbonate (500 mg, 3.59 mmol, 3.45 equiv) in DMF (15 mL) was stirred overnight at RT. The resulting solution was diluted with 100 mL of water and then extracted with 2×100 mL of DCM. The combined organic layers were dried (Na₂SO₄), filtered and concentrated under vacuum. The residue was by SiO₂ chromatography eluting with DCM/MeOH (100:6) to afford 140 mg of crude 3-[1-(2-methoxyethyl)piperidin-4-yl]-10-methyl-1H,3H,4H,6H-pyrano[4,3-c]isochromen-6-one as a red oil: MS calcd. for C₂₁H₂₈NO₄ [(M+H)⁺]: 358.2, obsd. 358.0.

step 2: A solution of 3-[1-(2-methoxyethyl)piperidin-4-yl]-10-methyl-1H,3H,4H,6H-pyrano[4,3-c]isochromen-6-one (140 mg, 0.39 mmol, 1.00 equiv) in a saturated ammonia solution in MeOH (10 mL) was stirred in a 30-mL sealed tube overnight at 120° C. The resulting mixture was cooled to RT and concentrated under vacuum. The crude product was purified by Prep-HPLC with the following conditions: Column, Sunfire C18 19×150, mobile phase, CH₃CN:NH₄CO₃/H₂O (10 mmol/L)=10%-65%, 20 min, Detector UV 254 nm to give 16.2 mg (12%) of 3-[1-(2-methoxyethyl)piperidin-4-yl]-10-methyl-1H,3H,4H,5H,6H-pyrano[4,3-c]isoquinolin-6-one as a white solid. ¹H-NMR (300 MHz, DMSO-d₆) δ ppm 11.19 (s, 1H), 8.13 (d, J=6.9 Hz, 1H), 7.47 (d, J=7.2 Hz, 1H), 7.32-7.27 (m, 1H), 5.14 (d, J=13.8 Hz, 1H), 4.92 (d, J=13.8 Hz, 1H), 3.46-3.35 (m, 3H), 3.24 (s, 3H), 3.01-2.88 (m, 2H), 2.58 (s, 3H), 2.50-2.49 (m, 4H), 2.10-1.87 (m, 3H), 1.58-1.20 (m, 4H). MS calcd. for C₂₁H₂₉N₂O₃ [(M+H)⁺]: 357.2, obsd. 357.1.

3-[1-(2-hydroxyethyl)-4-piperidyl]-10-methyl-1 3 4 5-tetrahydropyrano[4,3-c]isoquinolin-6-one (I-75) was prepared analogously except in step 1 2-bromo-1-methoxyethane was replaced with 2-bromo-1-hydroxy-ethane. The parent ion of I-75 was m/e=343.1.

3-(2-hydroxypropyl)-10-methyl-1 3 4 5-tetrahydropyrano[4,3-c]isoquinolin-6-one (I-76) was prepared analogously except in step 1 2-bromo-1-methoxyethane was replaced with 2-bromo-1-hydroxy-propanee. The parent ion of I-76 was m/e=274.10.

10-methyl-3-(1-tetrahydropyran-4-ylsulfonyl-4-piperidyl)-1 3 4 5-tetrahydropyrano[4,3-c]isoquinolin-6-one (I-74) was prepared analogously except in step 1, 10-methyl-3-(piperidin-4-yl)-1H,3H,4H,6H-pyrano[4,3-c]isochromen-6-one hydrochloride was sulfonylated with tetrahydro-2H-pyran-4-sulfonyl chloride. The parent ion of 1-74 was m/e=447.1.

### Example 71

### μHTS-TNKS-IWR2 TR-FRET Binding Assay (10 μL/well in BD1536-well plate, a single point)

### Reagents and Stock Solutions

Tankyrase 1 (TNKS1): 184.3 μM=5.2 mg/mL His6-TNKS1, MW=28.2 KDa (construct: 1088-1327, 1266M) in 20 mM Tris pH 8, 150 mM NaCl, 10% glycerol, and 0.5 mM TCEP. Alternatively, His6-tankyrase 2 (construct: 934-1166) (His6-TNKS2) or His6-PARP1 (full length) can be substituted for His6-TNKS 1.

Biotin-4-((1S,2R,6S,7R)-3,5-Dioxo-4-aza-tricyclo[5.2.1.0*2,6*]dec-8-en-4-yl)-N-(4-methyl-quinolin-8-yl)-benzamide (Biotin-IWR2): 10 mM Biotin-IWR2 stock in DMSO, stored at −20° C.
Positive control: 10 mM 2-(4-Trifluoromethyl-phenyl)-3,5,7,8-tetrahydro-thiopyrano[4,3-d]pyrimidin-4-one (XAV939) in DMSO, stored at −20° C.
Eu-Streptavidin: 38.1 μM (2.1 mg/mL) Eu-SA (Bio# Eu-2212, Lot# N 18001-BDHO2)
APC-anti-His Ab: 8.50 μM SL-APC, 8.26 μM anti-6His antibody-SureLight APC (Columia Bioscience, Catalog Number D3-1711, Lot Number N01010-AAH04)
Assay plate: BD 1536-well, clear/black plate (Catalog Number 353255)
NP-40: 10% NP-40 solution (PIERCE, Catalog Number 28324, Lot Number 97101671)

### Assay Buffer Preparation

Assay buffer 1a (AB1a) for TNKS dilution: 50 mM Tris, pH 7.4, 100 mM sodium chloride solution, 1 mM magnesium chloride solution, 1 mM DL-dithiothreitol solution, 0.2 mg/mL bovine serum albumin solution, 0.025% NP-40.
Assay buffer 1b (AB 1b) for Biotin-IWR2 dilution: 50 mM Tris, pH 7.4, 100 mM sodium chloride solution, 1 mM magnesium chloride solution, 1 mM DL-dithiothreitol solution, 0.2 mg/mL bovine serum albumin solution, 0.05% NP-40
Assay buffer 1c (AB 1c) for compound dilution: 50 mM Tris, pH 7.4, 100 mM sodium chloride solution, 1 mM magnesium chloride solution, 1 mM DL-dithiothreitol solution, 0.2 mg/mL bovine serum albumin solution
Assay buffer 2 (AB2) for Eu/APC: 50 mM Tris, pH 7.4, 100 mM sodium chloride solution, 1 mM magnesium chloride solution, 0.2 mg/mL bovine serum albumin solution

### Reagent Stock Solution Preparation

Prepare Biotinylated IWR2 stock solution (3.33× stock) for TOTL and compound wells: 200 nM Biotin-IWR2 in 5% DMSO/AB1b buffer

Prepare BLANK well stock solution: 5% DMSO/AB1b buffer
Prepare POSITIVE CONTROL well stock solution (3.33× stock): 200 nM XAV939 in 200 nM Biotin-IWR2/5% DMSO/AB1b buffer
Prepare TNKS1 stock solution (5× stock): 300 nM TNKS in AB1a buffer. (Alternatively, use TNKS2 or PARP1 stock solutions.)
Prepare Eu/APC stock solution (5× stock): 3.5 nM Eu-SA/50 nM APC-His6Ab in AB2 buffer.

### Assay Procedure

### Compound Preparations:

Add 25 μL/well 1.5% DMSO/AB1c buffer in each compound well to the compound concentration at 74 μM in 8.8% DMSO/AB1c buffer or in the 2 μL DMSO CONTROL wells (BLANK, TOTAL and POSITIVE wells) in the compound plate. Transfer 3 μL/well of above solution (solution 1,2,3) to an empty assay plate (BD1536-well plate) as follows:
TOTAL and cpd wells: Solution 1 (Biotin-IWR2):
BLANK wells: Solution 2 (No Biotin-IWR2):
POSITIVE CONTROL wells: Solution 3 (Biotin-IWR2XAV939)
Transfer 3 μL/well of the above diluted compound solutions or compound dilution buffer to the above assay plate. Add 2 μL/well of 300 nM TNKS stock solution (4) to every well in the above assay plate. Centrifuge the assay plate at 2100 rpm for 2 min. Incubate the assay plate at 26° C. for 30 minutes. Add 2 μL/well 3.5 nMEu/50 nM APC solution (5) to every well in the above assay plate. Centrifuge the assay plate at 2100 rpm for 2 min. Incubate the assay plate at 26° C. for 60 min. Read the assay plate immediately at excitation wavelength of 330 nM and emission wavelength of 615 and 665 nM in time resolved fluorescence mode.

### Final Assay Conditions

### Biotin-IWR2: 60 nM

### TNKS: 60 nM

### Eu-SA: 0.7 nM

### APC-His Ab: 10 nM

XAV939 (+ve control): 60 nM at ˜70% Inhibition
General Library compounds: 22.23 μM in 4% DMSO
Representative compound data for the tankyrase assays is in TABLE 1. Representative compound data for the PARP1 assay is in TABLE 2 (below) Values are in μM.

TABLE I

| | | | |
|---|---|---|---|
| Example | TNKS1 | TNKS2 | PARP1 |

TABLE 2

| | PARP1 |
|---|---|
| Compound | IC₅₀ (μM) |
| I-1 | 0.0918 |
| I-2 | 0.0343 |
| I-3 | 0.281 |
| I-4 | 0.345 |
| I-5 | 0.0829 |
| I-6 | 0.0582 |
| I-7 | 0.119 |
| I-8 | 0.176 |
| I-9 | 2.271 |
| I-10 | 0.0801 |
| I-11 | 0.0371 |
| I-12 | 0.0306 |
| I-13 | 2.669 |
| I-14 | 0.68 |
| I-15 | >50 |
| I-16 | 0.0356 |
| I-17 | 0.107 |
| I-18 | >50 |
| I-19 | 0.0735 |
| I-20 | 0.0611 |
| I-21 | 0.0159 |
| I-22 | >50 |
| I-23 | 2.032 |
| I-24 | 0.0634 |
| I-25 | 0.0677 |
| I-26 | 0.0987 |
| I-27 | 0.197 |
| I-28 | 1.925 |
| I-29 | 0.126 |
| I-30 | 0.0533 |
| I-31 | 1.787 |
| I-32 | 0.0818 |
| I-33 | 0.0948 |
| I-34 | 3.467 |
| I-35 | 0.0699 |
| I-36 | 0.0277 |
| I-37 | 0.104 |
| I-38 | 18.65 |
| I-39 | 0.0365 |
| I-40 | 0.222 |
| I-41 | 0.0469 |
| I-42 | 2.095 |
| I-43 | 0.876 |
| I-44 | 0.0673 |
| I-45 | 0.0783 |
| I-46 | 0.0871 |
| I-47 | 0.0636 |
| I-48 | 0.437 |
| I-49 | 3.048 |
| I-50 | 0.104 |
| I-51 | 0.363 |
| I-52 | 0.0328 |
| I-53 | >50 |
| I-54 | 0.115 |
| I-55 | 0.125 |
| I-56 | >50 |
| I-57 | 0.0386 |
| I-58 | 0.328 |
| I-59 | 0.28 |
| I-60 | 0.0797 |
| I-61 | 0.0259 |
| I-62 | 0.0381 |
| I-63 | 0.0526 |
| I-64 | 0.0441 |
| I-65 | 0.043 |
| I-66 | 0.0897 |
| I-67 | 0.070 |
| I-68 | 0.026 |

### Example 72

### Tankyrase 1 Assay

Inhibition of the Wnt stimulated TCF transcriptional activity by tankyrase inhibitors was determined utilizing a HEK293-TS 112 TCF reporter cell line. A Wnt-responsive luciferase reporter named TOPbrite was constructed by cloning the enhancer element of Super8xTOPFlash containing eight TCF/LEF binding sites into the pGL4.28 vector (Promega) upstream of the minimal promoter element, and selecting for hygromycin B resistance (50 μg/ml). Cells were seeded into 384-well plates in the presence of 0.5 ug/mL of Wnt3A at a density of 20,000 cells per well in 25 ul of F:12 DMEM media supplemented with 10% FBS and 2 mM Glutamax. Cells seeded without the addition of Wnt3A were used as background signal. Compounds of various concentrations were added to cells and incubated at 37 degrees with 5% CO2 for 16 hours. The assay was terminated with the addition of Promega Dual Glo kit per manufacturer's instructions. A ratio of TOPbrite Firefly Luciferase and SV40 Renillla Luciferase was calculated and the background from the neutral wells was subtracted yielding the final normalized measurement of TCF transcriptional activity. Compound IC₅₀s were determined by four-parameter curve fitting using GeneData software.

### Example 73

Pharmaceutical compositions of the subject Compounds for administration via several routes can be prepared as described in this Example.

Composition for Oral Administration (A)

| Ingredient | % wt./wt. |
|---|---|
| Active ingredient | 20.0% |
| Lactose | 79.5% |
| Magnesium stearate | 0.5% |

The ingredients are mixed and dispensed into capsules containing about 100 mg each; one capsule would approximate a total daily dosage.

Composition for Oral Administration (B)

| Ingredient | % wt./wt. |
|---|---|
| Active ingredient | 20.0% |
| Magnesium stearate | 0.5% |
| Crosscarmellose sodium | 2.0% |
| Lactose | 76.5% |
| PVP (polyvinylpyrrolidine) | 1.0% |

The ingredients are combined and granulated using a solvent such as methanol. The formulation is then dried and formed into tablets (containing about 20 mg of active compound) with an appropriate tablet machine.

Composition for Oral Administration (C)

| Ingredient | % wt./wt. | |
|---|---|---|
| Active compound | 1.0 | g |
| Fumaric acid | 0.5 | g |
| Sodium chloride | 2.0 | g |
| Methyl paraben | 0.15 | g |
| Propyl paraben | 0.05 | g |
| Granulated sugar | 25.5 | g |
| Sorbitol (70% solution) | 12.85 | g |
| Veegum K (Vanderbilt Co.) | 1.0 | g |
| Flavoring | 0.035 | ml |
| Colorings | 0.5 | mg |
| Distilled water | q.s. to 100 | ml |

The ingredients are mixed to form a suspension for oral administration.

Parenteral Formulation (D)

| Ingredient | % wt./wt. | |
|---|---|---|
| Active ingredient | 0.25 | g |
| Sodium Chloride | qs to make isotonic | |
| Water for injection to | 100 | ml |

The active ingredient is dissolved in a portion of the water for injection. A sufficient quantity of sodium chloride is then added with stirring to make the solution isotonic. The solution is made up to weight with the remainder of the water for injection, filtered through a 0.2 micron membrane filter and packaged under sterile conditions.

Suppository Formulation (E)

| Ingredient | % wt./wt. |
|---|---|
| Active ingredient | 1.0% |
| Polyethylene glycol 1000 | 74.5% |
| Polyethylene glycol 4000 | 24.5% |

The ingredients are melted together and mixed on a steam bath, and poured into molds containing 2.5 g total weight.

Topical Formulation (F)

| Ingredients | grams |
|---|---|
| Active compound | 0.2-2 |
| Span 60 | 2 |
| Tween 60 | 2 |
| Mineral oil | 5 |
| Petrolatum | 10 |
| Methyl paraben | 0.15 |
| Propyl paraben | 0.05 |
| BHA (butylated hydroxy | 0.01 |
| anisole) | |
| Water | q.s. 100 |

All of the ingredients, except water, are combined and heated to about 60° C. with stirring. A sufficient quantity of water at about 60° C. is then added with vigorous stirring to emulsify the ingredients, and water then added q.s. about 100 g.

The features disclosed in the foregoing description, or the following claims, expressed in their specific forms or in terms of a means for performing the disclosed function, or a method or process for attaining the disclosed result, as appropriate, may, separately, or in any combination of such features, be utilized for realizing the invention in diverse forms thereof.

The foregoing invention has been described in some detail by way of illustration and example, for purposes of clarity and understanding. It will be obvious to one of skill in the art that changes and modifications may be practiced within the scope of the appended claims. Therefore, it is to be understood that the above description is intended to be illustrative and not restrictive. The scope of the invention should, therefore, be determined not with reference to the above description, but should instead be determined with reference to the following appended claims, along with the full scope of equivalents to which such claims are entitled.

The patents, published applications, and scientific literature referred to herein establish the knowledge of those skilled in the art and are hereby incorporated by reference in their entirety to the same extent as if each was specifically and individually indicated to be incorporated by reference. Any conflict between any reference cited herein and the specific teachings of this specifications shall be resolved in favor of the latter. Likewise, any conflict between an art-understood definition of a word or phrase and a definition of the word or phrase as specifically taught in this specification shall be resolved in favor of the latter.

We claim:

1. A compound of the formula 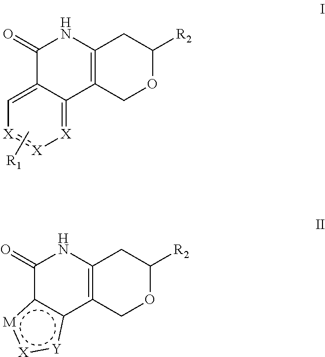
wherein
X is independently in each occurrence N or CH;
Y is S, O, CH or NCH₃;
M is S or CH;
R₁ is H, C₁₋₆alkyl, C₃₋₇ cycloalkyl, C(CH₃)₂OH, CN, NO₂, CO₂CH₃,CONH₂, NH₂, or halogen; and,
R₂ is selected from the group consisting of H, optionally substituted C₁₋₆ alkyl, C₅₋₁₂ spiroalkyl, C₁₋₆ alkoxy, C₃₋₇ cycloalkyl, heterocycloalkyl and substituted heterocycloalkyl wherein said heterocycloalkyl is optionally substituted by C₁₋₆ alkyl, C₁₋₆ hydroxyalkyl, C₁₋₃ alkoxy-C₁₋₆ alkyl, oxetanyl, tetrahydrofuranyl, pyranyl or SO₂R₃ wherein R₃ is C₁₋₆ alkyl, C₁₋₆ hydroxyalkyl, oxetanyl, tetrahydrofuranyl, pyranyl; or,
a pharmaceutically acceptable salt thereof.

2. The compound of claim 1 wherein: 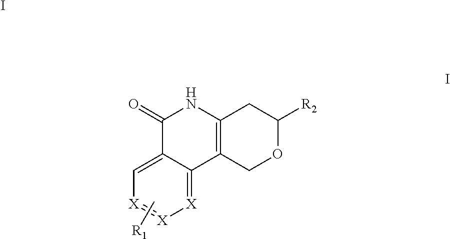
X is CH or N,
R₁ is H or CH₃ and
R₂ is selected from alkyl, substituted alkyl or substituted heterocycloalkyl.

3. The compound of claim 2 wherein said heterocycloalkyl is piperidin-4-yl optionally substituted by C₁₋₆ alkyl, C₁₋₆ hydroxyalkyl, C₁₋₃ alkoxy-C₁₋₆ alkyl, oxetanyl, tetrahydrofuranyl, pyranyl or SO₂R₃ wherein R₃ is C₁₋₆ alkyl, C₁₋₆ hydroxyalkyl, oxetanyl, tetrahydrofuranyl, pyranyl.

4. The compound of claim 1 selected from the group consisting of compounds I-1 to I-76 of TABLE 1

5. A pharmaceutical composition comprising a compound in accordance with claim 1 and at least one pharmaceutically acceptable carrier, diluent or excipient.

6. The use of a compound according to claim 1 for the treatment of cancer.

7. The use of a compound according to claim 1 for the preparation of a medicament for the treatment of cancer.

8. A method for the treatment of cancer, which method comprises administering an effective amount of a compound as defined in claim 1.
